(19) United States
(12) Patent Application Publication
(10) Pub. No.: US 2024/0260461 A1
(43) Pub. Date: Aug. 1, 2024

(54) ORGANIC ELECTROLUMINESCENT COMPOUND AND ORGANIC ELECTROLUMINESCENT DEVICE COMPRISING THE SAME

(71) Applicant: ROHM AND HAAS ELECTRONIC MATERIALS KOREA LTD., Chungcheongnam-do (KR)

(72) Inventors: Eun-Joung CHOI, Gyeonggi-do (KR); Mi-Ran SEO, Gyeonggi-do (KR); Young-Rok LIM, Gyeonggi-do (KR); HaeYeon KIM, Gyeonggi-do (KR); So-Mi PARK, Gyeonggi-do (KR); Dominea RATHWELL, Gyeonggi-do (KR)

(21) Appl. No.: 18/403,894

(22) Filed: Jan. 4, 2024

(30) Foreign Application Priority Data
Jan. 9, 2023 (KR) 10-2023-0002790
Dec. 18, 2023 (KR) 10-2023-0184899

(51) Int. Cl. H10K 85/60; C07D 401/04; C07D 405/14; C07D 471/04; C09K 11/06; H10K 50/13

(52) U.S. Cl. CPC H10K 85/6572; C07D 401/04; C07D 405/14; C07D 471/04; C09K 11/06; H10K 85/615; H10K 85/654; H10K 85/6574; C09K 2211/1018; H10K 50/131

(57) ABSTRACT

The present disclosure relates to an organic electroluminescent compound and an organic electroluminescent device comprising it. When the organic electroluminescent compound according to the present disclosure is included in a specific organic electroluminescent device, the organic electroluminescent device may exhibit a low driving voltage and/or improved lifespan properties.

### TECHNICAL FIELD

The present disclosure relates to an electroluminescent compound and an organic electroluminescent device comprising the same.

### BACKGROUND ART

Organic electroluminescent devices are self-illuminating display devices that have the advantages of a wide viewing angle, high contrast, and fast response. In organic electroluminescent devices, holes from the anode and electrons from the cathode are injected into the light-emitting layer by applying a voltage, and high-energy excitons are formed by the recombination of holes and electrons. This energy causes the organic light-emitting compound to be in an excited state, and as the excited state of the organic light-emitting compound returns to its ground state, the energy is released as emitted light.

In recent years, due to the potential of flat panel displays and general lighting devices, there is a continuous demand for the development of new materials. In order to improve the performance required by medium and large OLED panels, it is necessary to develop superior high-performance materials and more desirable device structures.

Korean Patent Publication No. 10-2027030 discloses an organic electroluminescent device comprising a triazine derivative compound as an organic layer material provided between a cathode and a light-emitting layer.

European Patent Application Laid-open No. EP 3670504 A1 discloses an organic electronic device comprising a triazinylquinoline derivative compound substituted with a phenyl derivative in an organic semiconducting layer provided between a light-emitting layer and a cathode.

Korean Patent Application Laid-open No. 10-2021-0069866 discloses an organic electroluminescent device comprising a triazinyl quinoline derivative compound substituted with a fluorenyl derivative as an organic layer material selected from the group consisting of a light emitting layer, an electron transport auxiliary layer, and an electron transport layer.

However, said references do not specifically disclose an organic electroluminescent device comprising a triazinyl quinoline derivative compound according to the present invention in an n-type charge generating layer located between pluralities of light-emitting units.

### DISCLOSURE OF THE INVENTION

### Problems to be Solved

The object of the present disclosure is, first, to provide an organic electroluminescent compound capable of providing an organic electroluminescent device exhibiting low driving voltage and/or improved lifespan properties, and second, to provide an organic electroluminescent device exhibiting low drive voltage and/or improved lifespan properties by comprising the organic electroluminescent compound herein.

### Solution to Problem

As a result of intensive studies to solve the technical problems above, the present inventors found that the aforementioned objective can be achieved by an organic electroluminescent device comprising a plurality of light-emitting units located between a first electrode and a second electrode and including at least one light-emitting layer(s); and at least one n-type charge generation layer(s) located between the adjacent light-emitting units, wherein the n-type charge generation layer comprises a triazinyl quinoline derivative compound, so that the present invention was completed.

### Advantageous Effects of Invention

The organic electroluminescent compound herein exhibits performance suitable for use in an organic electroluminescent device.

In addition, the organic electroluminescent device herein comprises the organic electroluminescent compound according to the present disclosure, thereby exhibiting low driving voltage and/or improved lifespan properties.

### BRIEF DESCRIPTION OF DRAWING

FIG. 1 illustrates an example of an organic electroluminescence compound according to one embodiment of the present disclosure.

FIG. 2 illustrates an example of an organic electroluminescence device according to one embodiment of the present disclosure.

FIG. 3 illustrates an example of an organic electroluminescence device according to another embodiment of the present disclosure.

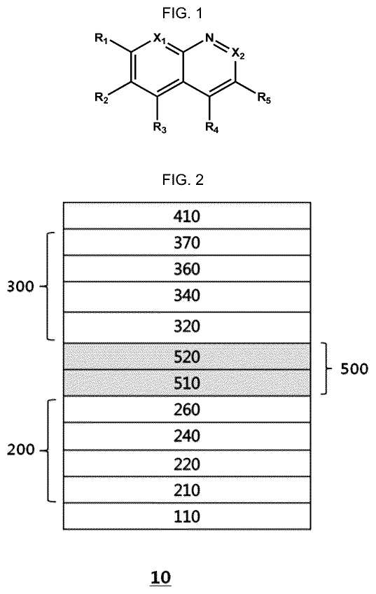

### EMBODIMENTS OF THE INVENTION

Hereinafter, the present disclosure will be described in detail. However, the following description is intended to explain the present disclosure, and is not meant to restrict the scope of the present disclosure.

The present disclosure relates to an organic electroluminescent device comprising at least one light-emitting unit(s) located between a first electrode and a second electrode; and at least one n-type charge generation layer(s) located between the adjacent light-emitting units.

The organic electroluminescent device according to the present disclosure comprises a triazinyl quinoline derivative compound in the n-type charge generation layer.

Herein, the term “organic electroluminescent compound” in the present disclosure refers to a compound that may be used in an organic electroluminescent device, and may be comprised in any material layer consisting of the organic electroluminescent device, as needed.

Herein, the term “organic electroluminescent material” in the present disclosure refers to a material that may be used in an organic electroluminescent device, and may comprise at least one compound(s). The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material (including a host material and a dopant material), an electron buffer material, a hole blocking material, an electron transport material, an electron injection material, etc.

Herein, the term “(C1-C30)alkyl(ene)” is meant to be a linear or branched alkyl having 1 to 30 carbon atoms constituting the chain, in which the number of carbon atoms is preferably 1 to 20, and more preferably 1 to 10. Examples of the alkyl may include methyl, ethyl, n-propyl, isopropyl, n-butyl, isobutyl, tert-butyl, sec-butyl, etc.

The term “(C3-C30)cycloalkyl(ene)” is meant to be a monocyclic or polycyclic hydrocarbon having 3 to 30 ring backbone carbon atoms, and preferably 3 to 20 ring backbone carbon atoms, more preferably 3 to 7 ring backbone carbon atoms. Examples of the cycloalkyl may include cyclopropyl, cyclobutyl, cyclopentyl, cyclohexyl, cyclopentylmethyl, cyclohexylmethyl, etc.

The term “(C6-C30)aryl(ene)” in the present disclosure is meant to refer to a monocyclic or fused ring radical derived from an aromatic hydrocarbon having 6 to 30 ring backbone carbon atoms, in which the number of ring backbone carbon atoms is preferably 6 to 20, and more preferably 6 to 15. The above aryl(ene) may be partially saturated, and may comprise a spiro structure. Examples of said aryl(ene) include phenyl, biphenyl, terphenyl, quaterphenyl, naphthyl, binaphthyl, phenylnaphthyl, naphthylphenyl, fluorenyl, phenylfluorenyl, dimethylfluorenyl, diphenylfluorenyl, benzofluorenyl, diphenylbenzofluorenyl, dibenzofluorenyl, phenanthrenyl, benzophenanthrenyl, phenylphenanthrenyl, anthracenyl, benzanthracenyl, indenyl, triphenylenyl, pyrenyl, tetracenyl, perylenyl, chrysenyl, benzocrysenyl, naphthacenyl, fluoranthenyl, benzofluoranthenyl, tolyl, xylyl, mesityl, cumenyl, spiro[fluorene-fluorene]yl, spiro[fluorene-benzofluorene]yl, azulenyl, and tetramethyl-dihydrophenanthrenyl. More specifically, examples of said aryl include o-tolyl, m-tolyl, p-tolyl, 2,3-xylyl, 3,4-xylyl, 2,5-xylyl, mesityl, o-cumenyl, m-cumenyl, p-cumenyl, p-t-butylphenyl, p-(2-phenylpropyl)phenyl, 4′-methylbiphenyl, 4″-t-butyl-p-terphenyl-4-yl, o-biphenyl, m-biphenyl, p-biphenyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 1-naphthyl, 2-naphthyl, 1-fluorenyl, 2-fluorenyl, 3-fluorenyl, 4-fluorenyl, 9-fluorenyl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 1-anthryl, 2-anthryl, 9-anthryl, 1-phenanthryl, 2-phenanthryl, 3-phenanthryl, 4-phenanthryl, 9-phenanthryl, 1-chrysenyl, 2-chrysenyl, 3-chrysene, 4-chrysene, 5-chrysene, 6-chrysene, benzo[c]phenanthryl, benzo[g]chrysene, 1-triphenylenyl, 2-triphenylenyl, 3-triphenylenyl, 4-triphenylenyl, 3-fluoranthenyl, 4-fluoranthenyl, 8-fluoranthenyl, 9-fluoranthenyl, benzofluoranthenyl, 11, 11-dimethyl-1-benzo[a]fluorenyl, 11,11-dimethyl-2-benzo[a]fluorenyl, 11, 11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11, 11-dimethyl-9-benzo[a]fluorenyl, 11,11-dimethyl-10-benzo[a]fluorenyl, 11,11-dimethyl-1-benzo[b]fluorenyl, 11, 11-dimethyl-2-benzo[b]fluorenyl, 11, 11-dimethyl-3-benzo[b]fluorenyl, 11,11-dimethyl-4-benzo[b]fluorenyl, 11,11-dimethyl-5-benzo[b]fluorenyl, 11, 11-dimethyl-6-benzo[b]fluorenyl, 11, 11-dimethyl-7-benzo[b]fluorenyl, 11,11-dimethyl-8-benzo[b]fluorenyl, 11, 11-dimethyl-9-benzo[b]fluorenyl, 11, 11-dimethyl-10-benzo[b]fluorenyl, 11, 11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11, 11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11, 11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11, 11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11, 11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11, 11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc.

Herein, the term “(3- to 30-membered)heteroaryl(ene)” in the present disclosure is meant to refer to an aryl or arylene having 3 to 30 ring backbone atoms and including at least one heteroatom(s) selected from the group consisting of B, N, O, S, Si, P, Se, Te, and Ge, in which the number of ring backbone atoms is preferably 3 to 30, and more preferably 5 to 20. The number of heteroatoms is preferably from 1 to 4, and may be a monocyclic ring or a fused ring condensed with at least one benzene ring(s), and may be partially saturated. In addition, the above heteroaryl or heteroarylene comprises one formed by linking at least one heteroaryl or aryl group(s) to a heteroaryl group via single bond(s), and may comprise a spiro structure. The above heteroaryl may include a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl such as benzofuranyl, benzothiophenyl, isobenzofuranyl, dibenzofuranyl, dibenzothiophenyl, dibenzoselenophenyl, benzofuroquinolinyl, benzofuroquinazolinyl, benzofuronaphthyridinyl, benzofuropyrimidinyl, naphthofuropyrimidinyl, benzothienoquinolinyl, benzothienoquinazolinyl, benzothienonaphthyridinyl, benzothienopyrimidinyl, naphthothienopyrimidinyl, pyrimidoindolyl, benzopyrimidoindolyl, benzofuropyrazinyl, naphthofuropyrazinyl, benzothienopyrazinyl, naphthothienopyrazinyl, pyrazinodiol, benzopyrazinodiol, benzopyrazinodiol, benzoimidazolyl, benzothiazolyl, benzoisothiazolyl, benzoisoxazolyl, benzooxazolyl, imidazopyridinyl, isoindolyl, indolyl, benzoindolyl, indazolyl, benzothiadiazolyl, quinolyl, isoquinolyl, cinnolyl, quinazolinyl, quinoxalinyl, carbazolyl, azacarbazolyl, benzocarbazolyl, dibenzocarbazolyl, phenoxazinyl, phenanthridinyl, phenanthrolinyl, benzodioxolyl, indolizidinyl, acridinyl, silafluorenyl, germafluorenyl, benzotriazolyl, phenazinyl, imidazopyridinyl, chromenoquinazolinyl, thiocromenoquinazolinyl, dimethylbenzopyrimidinyl, indolocarbazolyl, indenocarbazolyl, etc. More specifically, the heteroaryl may include 1-pyrrolyl, 2-pyrrolyl, 3-pyrrolyl, 2-pyridinyl, 3-pyridinyl, 4-pyridinyl, 2-pyrimidinyl, 4-pyrimidinyl, 5-pyrimidinyl, 6-pyrimidinyl, 1,2,3-triazine-4-yl, 1,2,4-triazine-3-yl, 1,3,5-triazine-2-yl, 1-imidazolyl, 2-imidazolyl, 1-pyrazolyl, 1-indolizidinyl, 2-indolizidinyl, 3-indolizidinyl, 5-indolizidinyl, 6-indolizidinyl, 7-indolizidinyl, 8-indolizidinyl, 2-imidazopyridinyl, 3-imidazopyridinyl, 5-imidazopyridinyl, 6-imidazopyridinyl, 7-imidazopyridinyl, 8-imidazopyridinyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolinyl, 3-quinolinyl, 4-quinolinyl, 5-quinolinyl, 6-quinolinyl, 7-quinolinyl, 8-quinolinyl, 1-isoquinolinyl, 3-isoquinolinyl, 4-isoquinolinyl, 5-isoquinolinyl, 6-isoquinolinyl, 7-isoquinolinyl, 8-isoquinolinyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazolyl-1-yl, azacarbazolyl-2-yl, azacarbazolyl-3-yl, azacarbazolyl-4-yl, azacarbazolyl-5-yl, azacarbazolyl-6-yl, azacarbazolyl-7-yl, azacarbazolyl-8-yl, azacarbazolyl-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-phenanthrolinyl, 2-phenanthrolinyl, 3-phenanthrolinyl, 4-phenanthrolinyl, 6-phenanthrolinyl, 7-phenanthrolinyl, 8-phenanthrolinyl, 9-phenanthrolinyl, 10-phenanthrolinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrole-1-yl, 2-methylpyrrole-3-yl, 2-methylpyrrole-4-yl, 2-methylpyrrole-5-yl, 3-methylpyrrole-1-yl, 3-methylpyrrole-2-yl, 3-methylpyrrole-4-yl, 3-methylpyrrole-5-yl, 2-t-butylpyrrole-4-yl, 3-(2-phenylpropyl)pyrrole-1-yl, 2-methyl-1-indolyl, 4-methyl-1-indolyl, 2-methyl-3-indolyl, 4-methyl-3-indolyl, 2-t-butyl-1-indolyl, 4-t-butyl-1-indolyl, 2-t-butyl-3-indolyl, 4-t-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1, 2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2, 1-b]-benzofuranyl, 2-naphtho-[2,1-b]-benzofuranyl, 3-naphtho-[2, 1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2, 1-b]-benzofuranyl, 9-naphtho-[2, 1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1, 2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2, 3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2, 1-b]-benzothiophenyl, 4-naphtho-[2, 1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2, 1-b]-benzothiophenyl, 10-naphtho-[2,1-b]-benzothiophenyl, 2-benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyrimidinyl, 8-benzothio[3,2-d]pyrimidinyl, 9-benzothio[3, 2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. In addition, “heteroaryl(ene)” may be classified into heteroaryl(ene) as having electron characteristics and heteroaryl(ene) having hole characteristics. The heteroaryl(ene) having electron characteristics is a substituent in which electrons are relatively abundant in the mother nucleus, and may be, for example, a substituted or unsubstituted pyridinyl, a substituted or unsubstituted pyrimidinyl, a substituted or unsubstituted triazinyl, a substituted or unsubstituted quinazolinyl, a substituted or unsubstituted quinoxalinyl, a substituted or unsubstituted quinolyl, etc. The heteroaryl(ene) having hole characteristics may be a substituent having relatively insufficient electrons in the mother nucleus, and may be, for example, a substituted or unsubstituted carbazolyl, a substituted or unsubstituted dibenzofuranyl, or a substituted or unsubstituted dibenzothiophenyl.

Herein, the term “a fused ring of a (C3-C30)aliphatic ring and a (C6-C30)aromatic ring” refers to a functional group of a ring fused with at least one aliphatic ring(s) having 3 to 30 ring backbone carbon atoms in which the number of carbon atoms is preferably 3 to 25, more preferably 3 to 18, and at least one aromatic ring(s) having 6 to 30 backbone carbon atoms in which the number of carbon atoms is preferably 6 to 25, and more preferably 6 to 18. For example, a fused ring of one or more benzene and one or more cyclohexane, or a fused ring of one or more naphthalene and one or more cyclopentane, etc. Herein, the carbon atoms of the fused ring of (C3-C30)aliphatic ring and (C6-C30)aromatic ring may be replaced by one or more heteroatoms selected from B, N, O, S, Si, and P, and preferably one or more heteroatoms selected from N, O, and S. Herein, the term “halogen” includes F, C1, Br, and I.

In addition, “ortho (o-),” “meta (m-),” and “para (p-)” are prefixes, which represent the relative positions of substituents, respectively. Ortho indicates that two substituents are adjacent to each other; for example, when two substituents in a benzene derivative occupy positions 1 and 2, it is called an ortho position. Meta indicates that two substituents are at positions 1 and 3; for example, when two substituents in a benzene derivative occupy positions 1 and 3, it is called a meta position. Para indicates that two substituents are at positions 1 and 4; for example, when two substituents in a benzene derivative occupy positions 1 and 4, it is called a para position.

Herein, “a ring formed in linking to an adjacent substituent” refers to a substituted or unsubstituted (3- to 50-membered) monocyclic or polycyclic alicyclic, aromatic, or combination thereof ring fused by connection or fusion of two or more adjacent substituents, preferably a substituted or unsubstituted (5- to 40-membered) monocyclic or polycyclic alicyclic, aromatic, or combination thereof ring. Further, the formed ring may comprise one or more heteroatoms selected from B, N, O, S, Si, and P, preferably one or more heteroatoms selected from N, O, and S. According to one embodiment of the present disclosure, the number of cyclic backbone atoms may be 5 to 35, and according to another embodiment of the present disclosure, the number of cyclic backbone atoms may be 5 to 30. In one embodiment, the fused ring is, for example, a substituted or unsubstituted fluorene ring, a substituted or unsubstituted dibenzothiophene ring, a substituted or unsubstituted dibenzofuran ring, a substituted or unsubstituted naphthalene ring, a substituted or unsubstituted phenanthrene ring, a substituted or unsubstituted benzofluorene ring, a substituted or unsubstituted benzothiophene ring, a substituted or unsubstituted benzofuran ring, a substituted or unsubstituted indole ring, a substituted or unsubstituted indene ring, a substituted or unsubstituted benzene ring, or a substituted or unsubstituted carbazole ring, etc.

In addition, in the “substituted or unsubstituted” description herein, the term “substituted” refers to a hydrogen atom in a functional group that is replaced with another atom or another functional group (i.e., a substituent). Unless otherwise specified, the substituents may not be limited to hydrogen at positions where the substituents may be substituted, and when two or more hydrogen atoms are each replaced with a substituent in a functional group, the substituents may be the same or different from each other. The maximum number of substituents that may be substituted with a certain functional group may be the total number of substituents that may be substituted with each atom constituting a functional group. Preferably, the substituted alkyl, the substituted aryl, the substituted heteroaryl, the substituted cycloalkyl, the substituted alkoxy, the substituted trialkylsilyl, the substituted dialkylarylsilyl, the substituted alkyldiarylsilyl, the substituted triarylsilyl, the substituted fused ring of an aliphatic ring and an aromatic ring, the substituted mono- or di-alkylamino, the substituted mono- or di-arylamino, the substituted alkylarylamino, the substituted mono- or di-heteroarylamino, and the substituted arylheteroarylamino, in the formulae herein, are each independently substituted with at least one selected from the group consisting of deuterium, halogen, cyano, carboxyl, nitro, hydroxyl, (C1-C30)alkyl, halo(C1-C30)alkyl, (C2-C30)alkenyl, (C2-C30)alkynyl, (C1-C30)alkoxy, (C1-C30)alkylthio, (C3-C30)cycloalkyl, (C3-C30)cycloalkenyl, (3- to 7-membered)heterocycloalkyl, (C6-C30)aryloxy, (C6-C30)arylthio, (5- to 30-membered)heteroaryl unsubstituted or substituted with (C6-C30)aryl, (C6-C30)aryl substituted or unsubstituted with (5- to 30-membered)heteroaryl, tri(C1-C30)alkylaryl, tri(C6-C30)arylsilyl, di(C1-C30)alkyl(C6-C30)arylsilyl, (C1-C30)alkyldi(C6-C30)arylsilyl, a fused ring of (C3-C30)aliphatic ring and (C6-C30)aromatic ring, amino, mono- or di-(C1-C30)alkylamino, a substituted or unsubstituted mono- or di-(C6-C30)arylamino, (C1-C30)alkyl(C6-C30)arylamino, mono- or di-(3- to 30-membered)heteroarylamino, (C1-C30)alkyl(3- to 30-membered) heteroarylamino, (C6-C30)aryl(3- to 30-membered) heteroarylamino, (C1-C30)alkylcarbonyl, (C1-C30)alkoxycarbonyl, (C6-C30)arylcarbonyl, (C6-C30)arylphosphinyl, di(C6-C30)arylboronyl, di(C1-C30)alkylboronyl, (C1-C30)alkyl(C6-C30)arylboronyl, (C6-C30)ar(C1-C30)alkyl, and (C1-C30)alkyl(C6-C30)aryl. For example, the substituent may be a substituted or unsubstituted methyl, a substituted or unsubstituted phenyl, a substituted or unsubstituted naphthyl, a substituted or unsubstituted biphenyl, a substituted or unsubstituted diphenylfluorenyl, a substituted or unsubstituted dimethylfluorenyl, a substituted or unsubstituted pyridinyl, a substituted or unsubstituted dibenzofuranyl, a substituted or unsubstituted dibenzothiophenyl, or a substituted or unsubstituted carbazolyl, etc.

When a substituent is not shown in the chemical formula or the compound structure, it may signify that all positions that may be present as substituents are hydrogen or deuterium. That is, in the case of deuterium, an isotope of hydrogen, some of the hydrogen atoms may be deuterium, which is an isotope; and in this case, the content of deuterium may be 0% to 100%. In the case where the substituent is not shown in the chemical formula or the compound structure, when deuterium is not explicitly excluded, hydrogen and deuterium may be mixed and used in the compound, such as when the content of deuterium is 0%, the content of hydrogen is 100%, and all substituents are hydrogen. The deuterium is an element having a deuteron composed of one proton and one neutron as an atomic nucleus, which is one of the isotopes of hydrogen, and may be represented by hydrogen-2, and the element symbol may be D or 2H. Although the isotope has the same atomic number (Z), an isotope having a different mass number (A) means the same number of protons and the number of neutrons may also be interpreted as an element having different numbers.

Herein, “combinations thereof” signifies that one or more components of the corresponding list are combined to form a known or chemically stable arrangement that a person skilled in the art could conceive of from the corresponding list. For example, alkyl and deuterium may be combined to form partially or entirely deuterated alkyl groups; halogen and alkyl may be combined to form halogenated alkyl substituents; and halogen, alkyl, and aryl may be combined to form halogenated arylalkyl. For example, preferred combinations of substituents may include up to 50 atoms excluding hydrogen and deuterium, or include up to 40 atoms excluding hydrogen and deuterium, or include up to 30 atoms excluding hydrogen and deuterium, or in many cases, preferred combinations of substituents may include up to 20 atoms excluding hydrogen and deuterium.

In the formula of the present disclosure, when multiple substituents are indicated by the same symbol, each of these substituents represented by the same symbol may be the identical or different from one another.

Identical drawing symbols refer to identical components. In addition, in the drawings, the thicknesses, proportions, and dimensions of the components might be exaggerated for an effective illustration of the technical content, but this does not result in a change in the technical content.

Hereinafter, an organic electroluminescent compound is described in accordance with one embodiment.

According to one embodiment of the present disclosure, the organic electroluminescent compound of the present disclosure is an organic electroluminescent compound represented by the following formula 1.

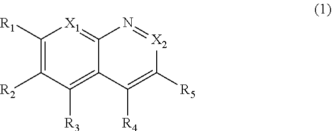

In formula 1,
R₁ to R₅ each independently represent, hydrogen, deuterium, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, or a substituted or unsubstituted tri(C6-C30)arylsilyl;
X₁ and X₂ each independently represent CR₈ or are represented by the following formula 2, and one of X₁ and X₂ is represented by the following formula 2, with the proviso that the case where both X₁ and X₂ are represented by the following formula 2 is excluded; and
R₈ represents hydrogen, deuterium, or a substituted or unsubstituted (C6-C30)aryl.

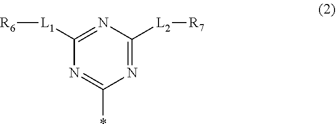

In formula 2,
L₁ and L₂ each independently represent a single bond, a substituted or unsubstituted (C6-C30) arylene, a substituted or unsubstituted (3- to 30-membered)heteroarylene, or a substituted or unsubstituted (C3-C30)cycloalkylene;
R₆ and R₇ each independently represent a substituted or unsubstituted (C1-C30)alkyl, a substituted or unsubstituted (C6-C30)aryl, a substituted or unsubstituted (3-to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, or a substituted or unsubstituted tri(C6-C30)arylsilyl; and
* represents a carbon atom attached to formula 1.

In one embodiment, R₁ to R₅ may each be, independently hydrogen, deuterium, or a substituted or unsubstituted (C6-C30)aryl, preferably, hydrogen, deuterium, or a substituted or unsubstituted (C6-C25)aryl, and more preferably, hydrogen, deuterium, or a substituted or unsubstituted (C6-C18)aryl. For example, R₁ to R₅ may each be, independently hydrogen, deuterium, a substituted or unsubstituted phenyl, or a substituted or unsubstituted naphthyl, and the substituted phenyl or the substituted naphthyl may be substituted with at least one selected from deuterium and a phenyl substituted with deuterium.

In one embodiment, L₁ and L₂ may each be, independently a single bond, a substituted or unsubstituted (C6-C30)arylene, or a substituted or unsubstituted (3- to 30-membered)heteroarylene, preferably, a single bond, a substituted or unsubstituted (C6-C25)arylene, or a substituted or unsubstituted (5- to 25-membered)heteroarylene, more preferably, a single bond, a substituted or unsubstituted (C6-C18)arylene, or a substituted or unsubstituted (5- to 18-membered)heteroarylene. For example, L₁ and L₂ may each be, independently a single bond, a substituted or unsubstituted phenylene, a substituted or unsubstituted naphthylene, or a substituted or unsubstituted biphenylene, and the substituted phenylene, the substituted naphthylene, or the substituted biphenyl may be substituted with deuterium.

In one embodiment, R₆ and R₇ may each be, independently a substituted or unsubstituted (C6-C30)aryl, or a substituted or unsubstituted (3- to 30-membered)heteroaryl, preferably, a substituted or unsubstituted (C6-C25)aryl, or a substituted or unsubstituted (5- to 25-membered)heteroaryl, more preferably, a substituted or unsubstituted (C6-C18)aryl, or a substituted or unsubstituted (5- to 18-membered)heteroaryl. For example, R₆ and R₇ may each be, independently a substituted or unsubstituted phenyl, a substituted or unsubstituted naphthyl, a substituted or unsubstituted dibenzofuranyl, a substituted or unsubstituted dibenzothiophenyl, a substituted or unsubstituted biphenyl, a substituted or unsubstituted m-terphenyl, a substituted or unsubstituted o-terphenyl, a substituted or unsubstituted dimethylfluorenyl, a substituted or unsubstituted diphenylfluorenyl, a substituted or unsubstituted phenanthrenyl, a substituted or unsubstituted phenanthrolinyl, a substituted or unsubstituted anthracenyl, a substituted or unsubstituted chrysenyl, a substituted or unsubstituted isocrysenyl, a substituted or unsubstituted benzoxazolyl, or a substituted or unsubstituted carbazolyl. The substituted phenyl, the substituted naphthyl, the substituted dibenzofuranyl, the substituted dibenzothiophenyl, the substituted biphenyl, the substituted m-terphenyl, the substituted o-terphenyl, the substituted dimethylfluorenyl, the substituted diphenylfluorenyl, the substituted phenanthrenyl, or the substituted chrysenyl may be substituted with deuterium, the substituted phenanthrolinyl may be substituted with at least one selected from phenyl and naphthyl, and the substituted anthracenyl, the substituted benzooxazolyl, or the substituted carbazolyl may be substituted with phenyl.

In one embodiment, R₈ may be hydrogen, deuterium, or a substituted or unsubstituted (C6-C30)aryl, preferably, hydrogen, deuterium, or a substituted or unsubstituted (C6-C25)aryl, more preferably, hydrogen, deuterium, or a substituted or unsubstituted (C6-C18)aryl. For example, R₈ may be hydrogen, deuterium, or a substituted or unsubstituted phenyl, and the substituted phenyl may be substituted with deuterium.

According to one embodiment, the compound represented by the above formula 1 may be represented by the following formula 3.

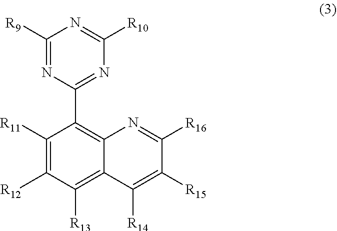

In formula 3,
R₉ to R₁₆ each independently represent, hydrogen, deuterium, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, or a substituted or unsubstituted tri(C6-C30)arylsilyl; and

With the proviso that neither R₉ nor R₁₀ is a substituted or unsubstituted fluorenyl. In one embodiment, R₉ to R₁₆ may each be, independently a substituted or unsubstituted (C6-C30)aryl, or substituted or unsubstituted (3- to 30-membered)heteroaryl, preferably, a substituted or unsubstituted (C6-C25)aryl, or a substituted or unsubstituted (5- to 25-membered)heteroaryl, more preferably, a substituted or unsubstituted (C6-C18)aryl, or a substituted or unsubstituted (5- to 18-membered)heteroaryl. For example, R₉ to R₁₆ may each be, independently a substituted or unsubstituted phenyl, a substituted or unsubstituted naphthyl, a substituted or unsubstituted dibenzofuranyl, a substituted or unsubstituted dibenzothiophenyl, a substituted or unsubstituted biphenyl, a substituted or unsubstituted anthracenyl, a substituted or unsubstituted phenanthrenyl, a substituted or unsubstituted phenanthrolinyl, a substituted or unsubstituted chrysenyl, or a substituted or unsubstituted benzoxazolyl. The substituted phenyl, the substituted naphthyl, the substituted dibenzofuranyl, the substituted dibenzothiophenyl, the substituted phenanthrenyl, or the substituted chrysenyl may be substituted with deuterium, and the substituted anthracenyl, the substituted phenanthrolinyl, or the substituted benzoxazolyl may be substituted with phenyl.

In one embodiment, at least one of R₉ to R₁₆ may be a substituted or unsubstituted (C10-C30) fused ring-based aryl or a substituted or unsubstituted (10- to 30-membered) fused ring-based heteroaryl, preferably, a substituted or unsubstituted (C10-C25) fused ring-based aryl or a substituted or unsubstituted (10- to 25-membered) fused ring-based heteroaryl, more preferably, a substituted or unsubstituted (C10-C22) fused ring-based aryl or a substituted or unsubstituted (10- to 22-membered) fused ring-based heteroaryl. For example, at least one of R₉ to R₁₆ may be a substituted or unsubstituted naphthyl, a substituted or unsubstituted dibenzofuranyl, a substituted unsubstituted dibenzothiophenyl, a substituted or unsubstituted anthracenyl, a substituted or unsubstituted phenanthrenyl, a substituted or unsubstituted chrysenyl, or a substituted or unsubstituted benzoxazolyl, and the substituted naphthyl, the substituted dibenzofuranyl, the substituted phenanthrenyl, or the substituted chrysenyl may be substituted with deuterium, and the substituted anthracenyl or the substituted benzoxazolyl may be substituted with phenyl.

According to one embodiment, the compound represented by the above formula 1 may be represented by the following formula 4.

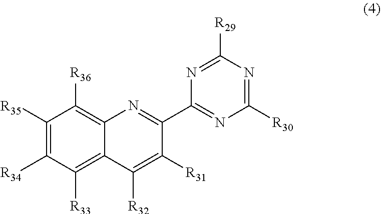

In formula 4,
R₂₉ to R₃₆ each independently represent, hydrogen, deuterium, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, or a substituted or unsubstituted tri(C6-C30)arylsilyl; and

With the proviso that neither R₂₉ nor R₃₀ is a substituted or unsubstituted fluorenyl.

In one embodiment, R₂₉ to R₃₆ may each be, independently hydrogen, deuterium, a substituted or unsubstituted (C6-C30)aryl, or a substituted or unsubstituted (3- to 30-membered)heteroaryl, preferably, hydrogen, deuterium, a substituted or unsubstituted (C6-C25)aryl, or a substituted or unsubstituted (3- to 25-membered)heteroaryl, more preferably, hydrogen, deuterium, a substituted or unsubstituted (C6-C18)aryl, or a substituted or unsubstituted (3- to 18-membered)heteroaryl. For example, R₂₉ to R₃₆ may each be, independently a substituted or unsubstituted phenyl, a substituted or unsubstituted naphthyl, a substituted or unsubstituted phenanthrenyl, a substituted or unsubstituted phenanthrolinyl, a substituted or unsubstituted anthracenyl, a substituted or unsubstituted isocrysenyl, a substituted or unsubstituted biphenyl, a substituted or unsubstituted m-terphenyl, a substituted or unsubstituted dibenzofuranyl, a substituted or unsubstituted dibenzothiophenyl, or a substituted or unsubstituted carbazolyl. The substituted phenyl, the substituted naphthyl, the substituted phenanthrenyl, the substituted biphenyl, the substituted dibenzofuranyl, or the substituted dibenzothiophenyl may be substituted with deuterium, the substituted naphthyl or the substituted phenanthrolinyl may be substituted with phenyl, and the naphthyl substituted with phenyl may be substituted with deuterium.

According to one embodiment, the compound represented by formula 1 may be more specifically illustrated by the following compounds, but is not limited thereto.

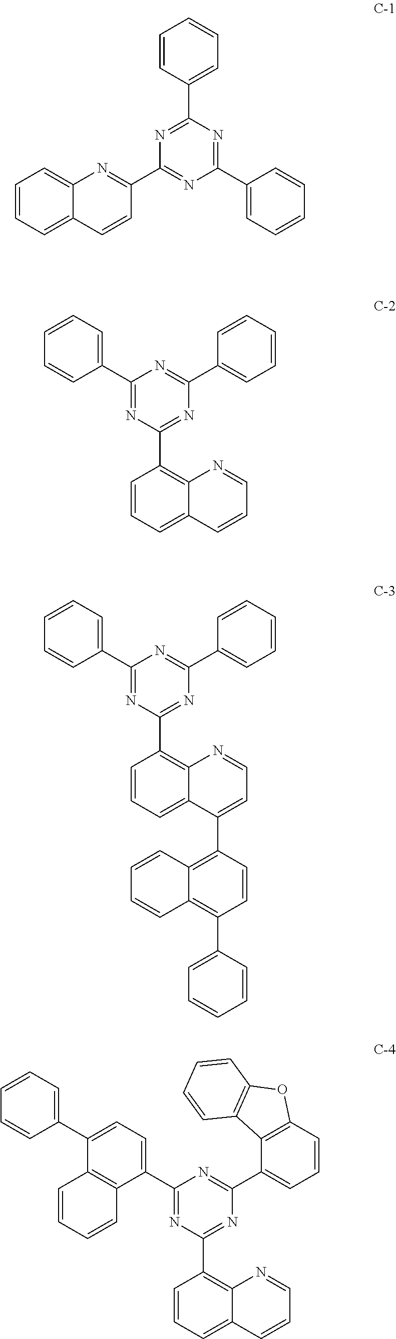

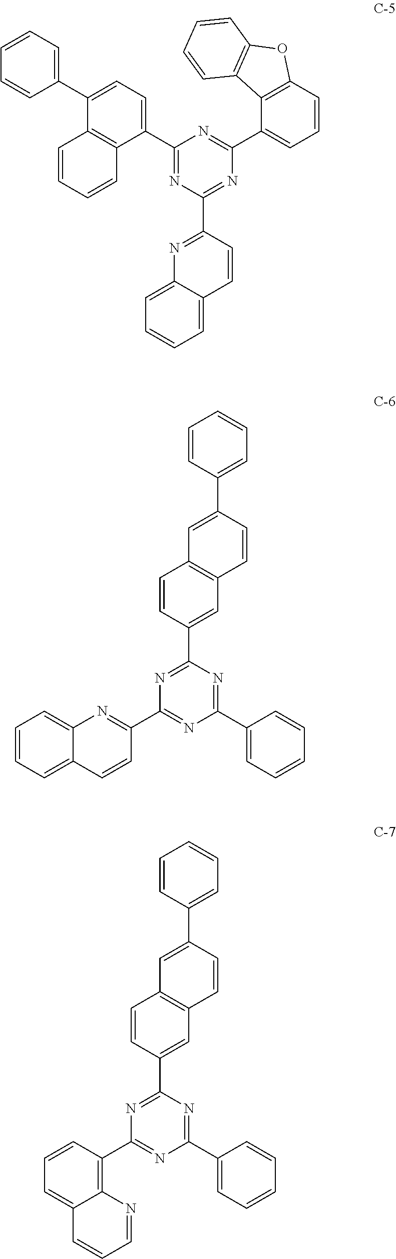 [SMILES: c1ccc(-c2ccc(-c3nc(-c4ccc5ccccc5n4)nc(-c4cccc5oc6ccccc6c45)n3)c3ccccc23)cc1 ; c1ccc(-c2ccc3cc(-c4nc(-c5ccccc5)nc(-c5ccc6ccccc6n5)n4)ccc3c2)cc1 ; c1ccc(-c2ccc3cc(-c4nc(-c5ccccc5)nc(-c5cccc6cccnc56)n4)ccc3c2)cc1]

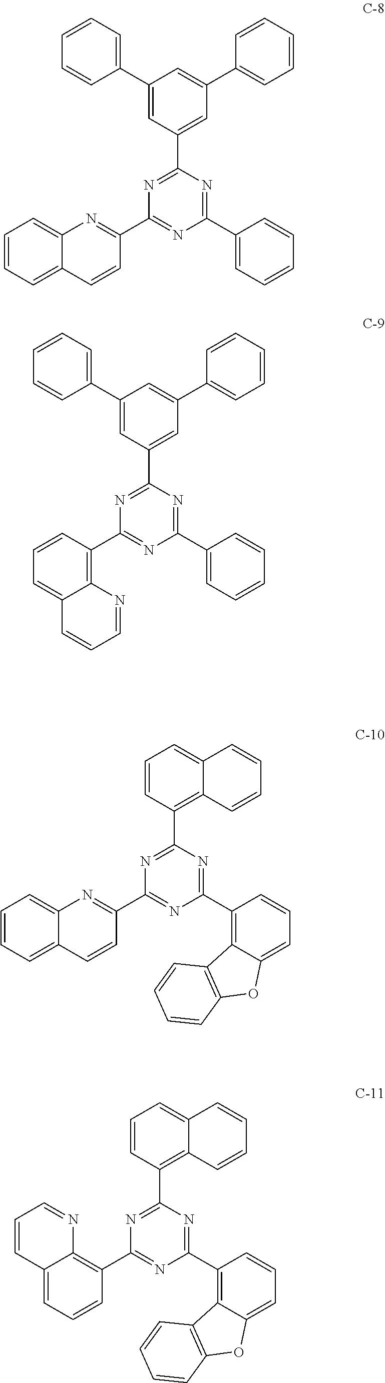 [SMILES: c1ccc(-c2cc(-c3ccccc3)cc(-c3nc(-c4ccccc4)nc(-c4ccc5ccccc5n4)n3)c2)cc1 ; c1ccc(-c2cc(-c3ccccc3)cc(-c3nc(-c4ccccc4)nc(-c4cccc5cccnc45)n3)c2)cc1 ; c1ccc2nc(-c3nc(-c4cccc5ccccc45)nc(-c4cccc5oc6ccccc6c45)n3)ccc2c1 ; c1ccc2c(-c3nc(-c4cccc5cccnc45)nc(-c4cccc5oc6ccccc6c45)n3)cccc2c1]

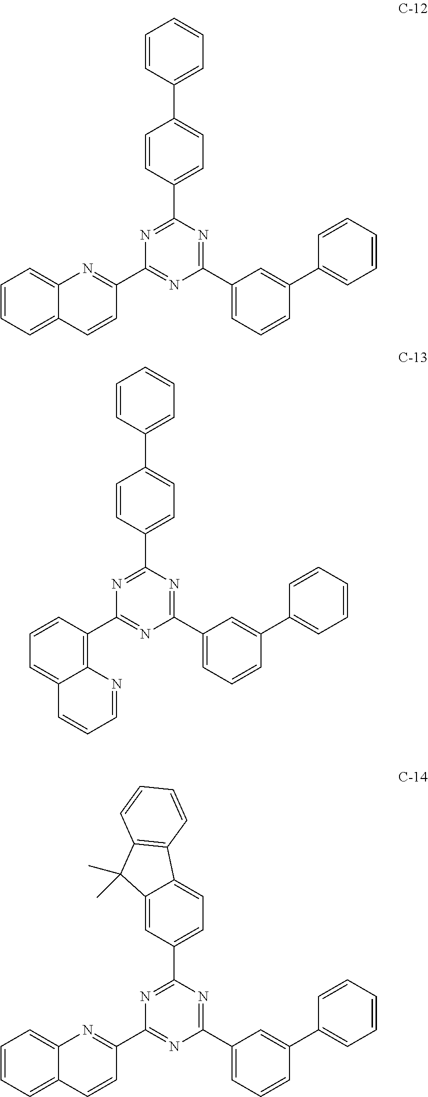 [SMILES: c1ccc(-c2ccc(-c3nc(-c4cccc(-c5ccccc5)c4)nc(-c4ccc5ccccc5n4)n3)cc2)cc1 ; c1ccc(-c2ccc(-c3nc(-c4cccc(-c5ccccc5)c4)nc(-c4cccc5cccnc45)n3)cc2)cc1 ; CC1(C)c2ccccc2-c2ccc(-c3nc(-c4cccc(-c5ccccc5)c4)nc(-c4ccc5ccccc5n4)n3)cc21]

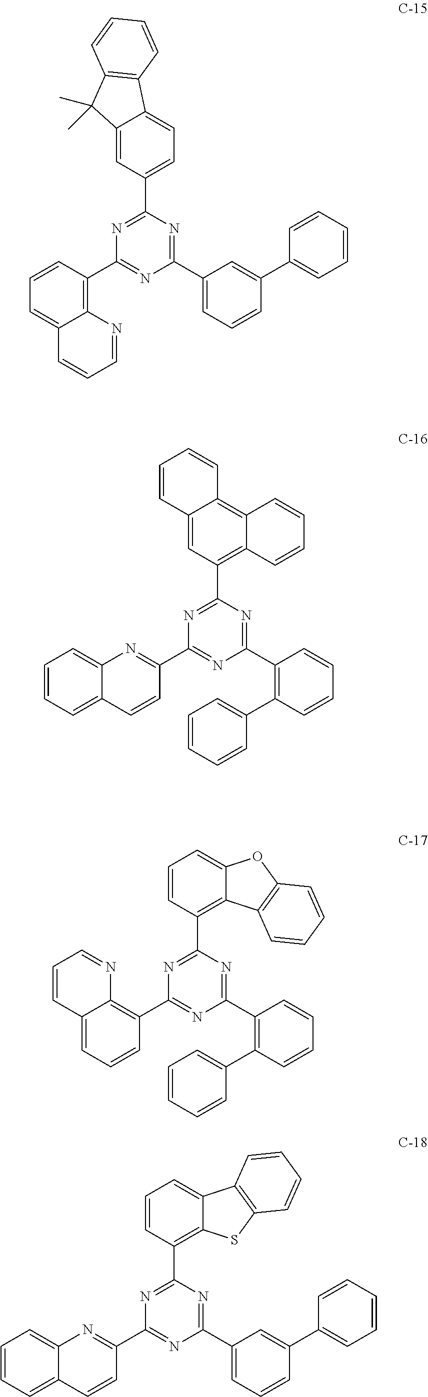 [SMILES: CC1(C)c2ccccc2-c2ccc(-c3nc(-c4cccc(-c5ccccc5)c4)nc(-c4cccc5cccnc45)n3)cc21 ; c1ccc(-c2ccccc2-c2nc(-c3ccc4ccccc4n3)nc(-c3cc4ccccc4c4ccccc34)n2)cc1 ; c1ccc(-c2ccccc2-c2nc(-c3cccc4cccnc34)nc(-c3cccc4oc5ccccc5c34)n2)cc1 ; c1ccc(-c2cccc(-c3nc(-c4ccc5ccccc5n4)nc(-c4cccc5c4sc4ccccc45)n3)c2)cc1]

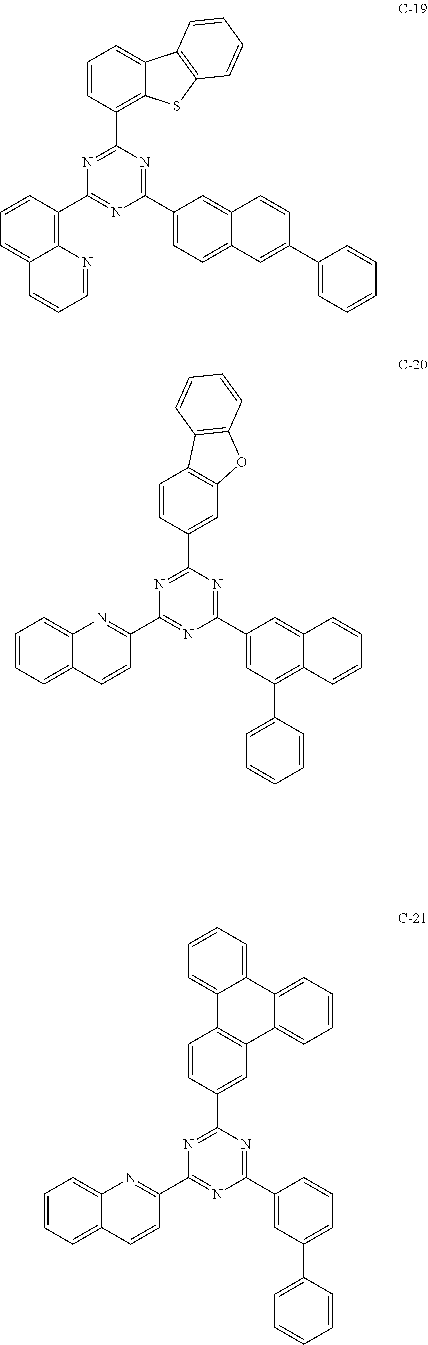 [SMILES: c1ccc(-c2ccc3cc(-c4nc(-c5cccc6cccnc56)nc(-c5cccc6c5sc5ccccc56)n4)ccc3c2)cc1 ; c1ccc(-c2cc(-c3nc(-c4ccc5c(c4)oc4ccccc45)nc(-c4ccc5ccccc5n4)n3)cc3ccccc23)cc1 ; c1ccc(-c2cccc(-c3nc(-c4ccc5c6ccccc6c6ccccc6c5c4)nc(-c4ccc5ccccc5n4)n3)c2)cc1]

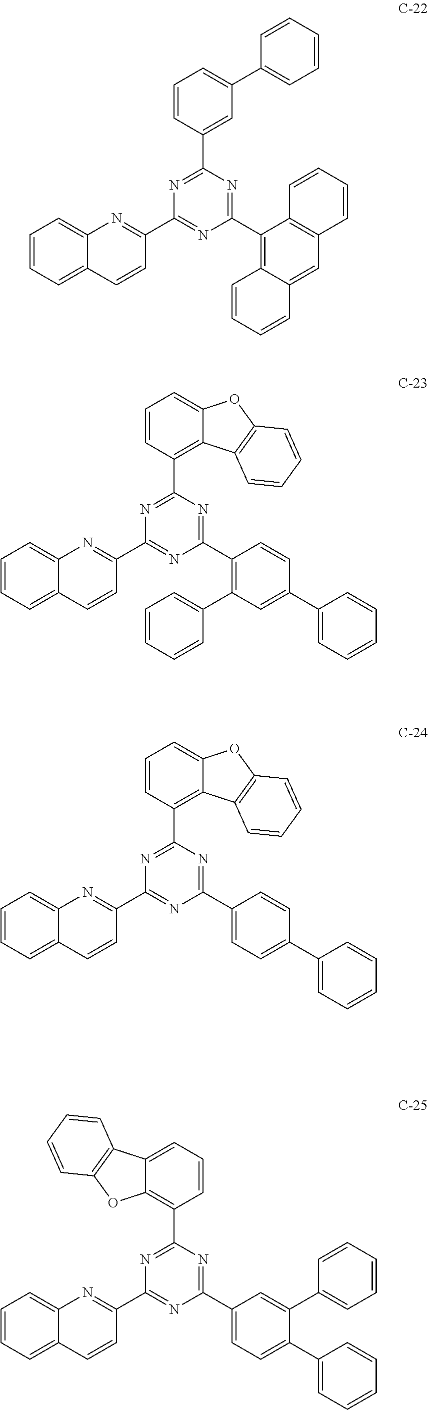 [SMILES: c1ccc(-c2cccc(-c3nc(-c4ccc5ccccc5n4)nc(-c4c5ccccc5cc5ccccc45)n3)c2)cc1 ; c1ccc(-c2ccc(-c3nc(-c4ccc5ccccc5n4)nc(-c4cccc5oc6ccccc6c45)n3)c(-c3ccccc3)c2)cc1 ; c1ccc(-c2ccc(-c3nc(-c4ccc5ccccc5n4)nc(-c4cccc5oc6ccccc6c45)n3)cc2)cc1 ; c1ccc(-c2ccc(-c3nc(-c4ccc5ccccc5n4)nc(-c4cccc5c4oc4ccccc45)n3)cc2-c2ccccc2)cc1]

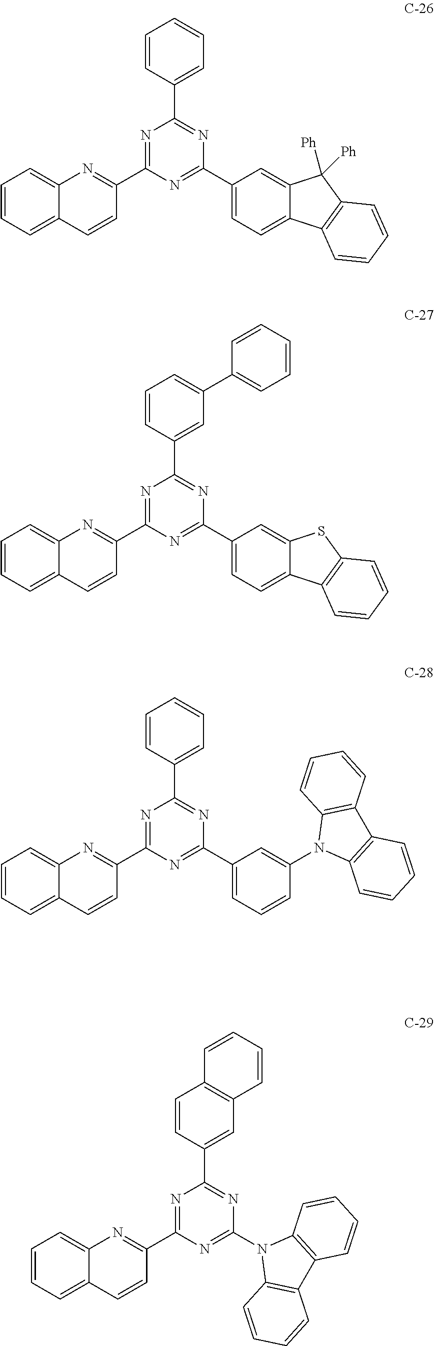 [SMILES: c1ccc(-c2nc(-c3ccc4c(c3)C(c3ccccc3)(c3ccccc3)c3ccccc3-4)nc(-c3ccc4ccccc4n3)n2)cc1 ; c1ccc(-c2cccc(-c3nc(-c4ccc5c(c4)sc4ccccc45)nc(-c4ccc5ccccc5n4)n3)c2)cc1 ; c1ccc(-c2nc(-c3cccc(-n4c5ccccc5c5ccccc54)c3)nc(-c3ccc4ccccc4n3)n2)cc1 ; c1ccc2cc(-c3nc(-c4ccc5ccccc5n4)nc(-n4c5ccccc5c5ccccc54)n3)ccc2c1]

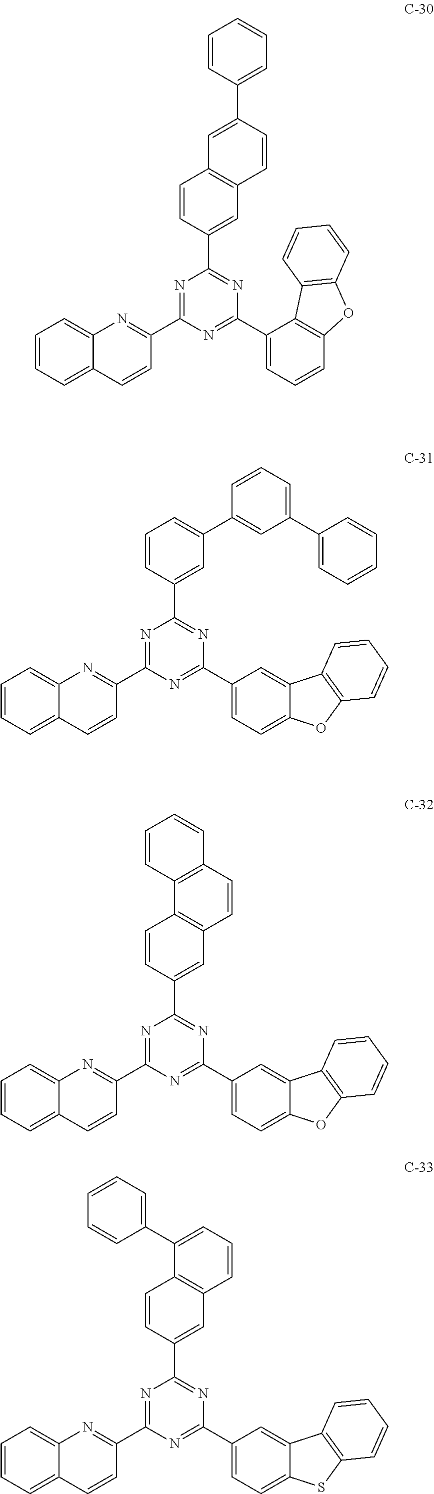 [SMILES: c1ccc(-c2ccc3cc(-c4nc(-c5ccc6ccccc6n5)nc(-c5cccc6oc7ccccc7c56)n4)ccc3c2)cc1 ; c1ccc(-c2cccc(-c3cccc(-c4nc(-c5ccc6oc7ccccc7c6c5)nc(-c5ccc6ccccc6n5)n4)c3)c2)cc1 ; c1ccc2nc(-c3nc(-c4ccc5c(ccc6ccccc65)c4)nc(-c4ccc5oc6ccccc6c5c4)n3)ccc2c1 ; c1ccc(-c2cccc3cc(-c4nc(-c5ccc6sc7ccccc7c6c5)nc(-c5ccc6ccccc6n5)n4)ccc23)cc1]

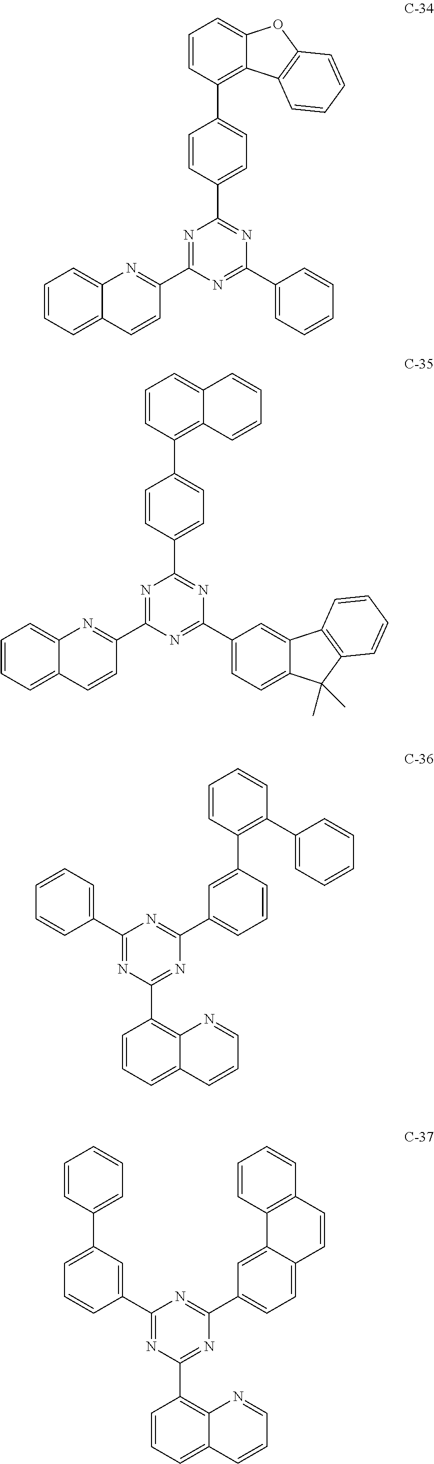 [SMILES: c1ccc(-c2nc(-c3ccc(-c4cccc5oc6ccccc6c45)cc3)nc(-c3ccc4ccccc4n3)n2)cc1 ; CC1(C)c2ccccc2-c2cc(-c3nc(-c4ccc(-c5cccc6ccccc56)cc4)nc(-c4ccc5ccccc5n4)n3)ccc21 ; c1ccc(-c2nc(-c3cccc(-c4ccccc4-c4ccccc4)c3)nc(-c3cccc4cccnc34)n2)cc1 ; c1ccc(-c2cccc(-c3nc(-c4ccc5ccc6ccccc6c5c4)nc(-c4cccc5cccnc45)n3)c2)cc1]

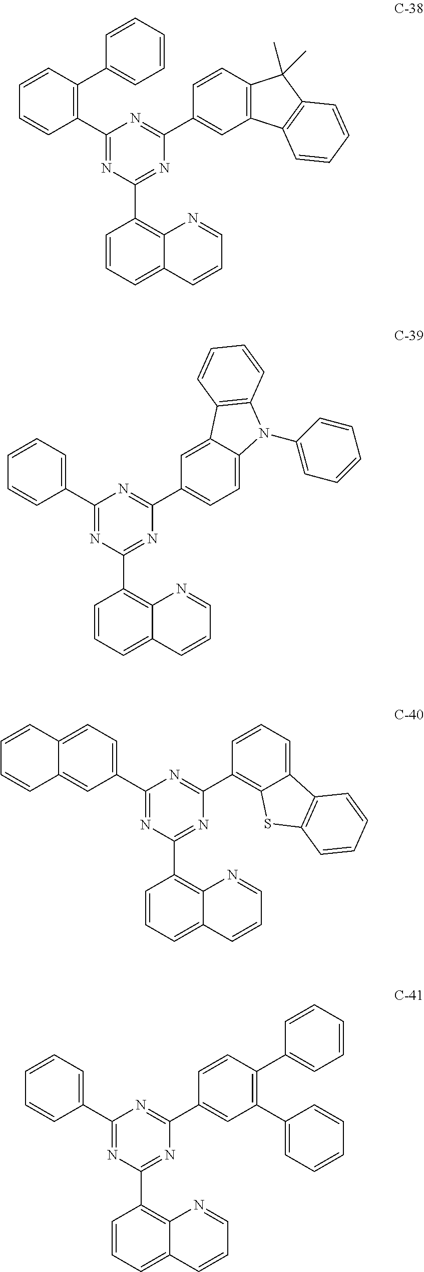 [SMILES: CC1(C)c2ccccc2-c2cc(-c3nc(-c4ccccc4-c4ccccc4)nc(-c4cccc5cccnc45)n3)ccc21 ; c1ccc(-c2nc(-c3ccc4c(c3)c3ccccc3n4-c3ccccc3)nc(-c3cccc4cccnc34)n2)cc1 ; c1ccc2cc(-c3nc(-c4cccc5cccnc45)nc(-c4cccc5c4sc4ccccc45)n3)ccc2c1 ; c1ccc(-c2nc(-c3ccc(-c4ccccc4)c(-c4ccccc4)c3)nc(-c3cccc4cccnc34)n2)cc1]

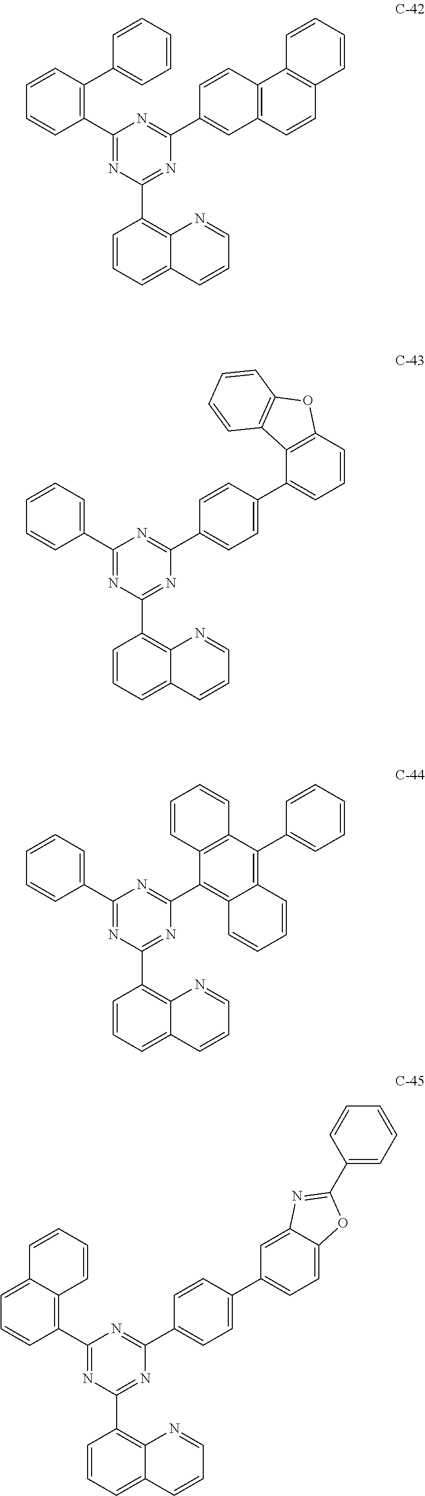 [SMILES: c1ccc(-c2ccccc2-c2nc(-c3ccc4c(ccc5ccccc54)c3)nc(-c3cccc4cccnc34)n2)cc1 ; c1ccc(-c2nc(-c3ccc(-c4cccc5oc6ccccc6c45)cc3)nc(-c3cccc4cccnc34)n2)cc1 ; c1ccc(-c2nc(-c3c4ccccc4c(-c4ccccc4)c4ccccc34)nc(-c3cccc4cccnc34)n2)cc1 ; c1ccc(-c2nc3cc(-c4ccc(-c5nc(-c6cccc7ccccc67)nc(-c6cccc7cccnc67)n5)cc4)ccc3o2)cc1]

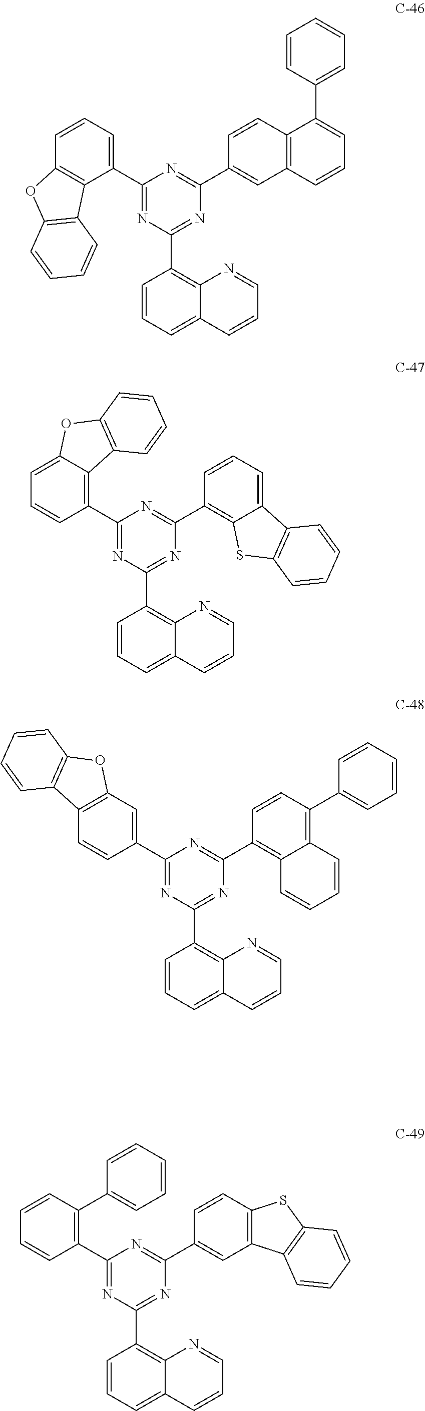 [SMILES: c1ccc(-c2cccc3cc(-c4nc(-c5cccc6cccnc56)nc(-c5cccc6oc7ccccc7c56)n4)ccc23)cc1 ; c1cnc2c(-c3nc(-c4cccc5c4sc4ccccc45)nc(-c4cccc5oc6ccccc6c45)n3)cccc2c1 ; c1ccc(-c2ccc(-c3nc(-c4ccc5c(c4)oc4ccccc45)nc(-c4cccc5cccnc45)n3)c3ccccc23)cc1 ; c1ccc(-c2ccccc2-c2nc(-c3ccc4sc5ccccc5c4c3)nc(-c3cccc4cccnc34)n2)cc1]

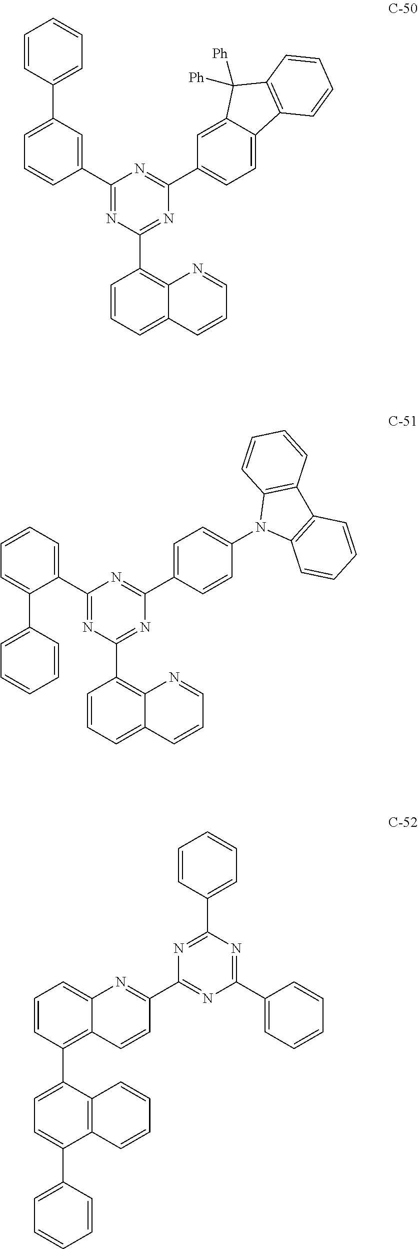 [SMILES: c1ccc(-c2cccc(-c3nc(-c4ccc5c(c4)C(c4ccccc4)(c4ccccc4)c4ccccc4-5)nc(-c4cccc5cccnc45)n3)c2)cc1 ; c1ccc(-c2ccccc2-c2nc(-c3ccc(-n4c5ccccc5c5ccccc54)cc3)nc(-c3cccc4cccnc34)n2)cc1 ; c1ccc(-c2nc(-c3ccccc3)nc(-c3ccc4c(-c5ccc(-c6ccccc6)c6ccccc56)cccc4n3)n2)cc1]

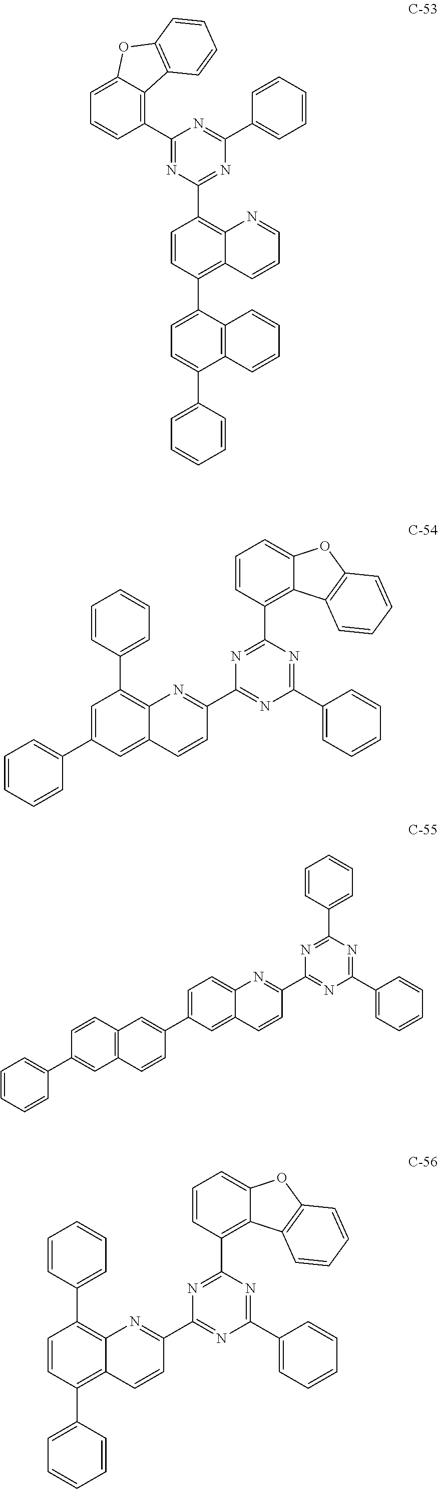 [SMILES: c1ccc(-c2nc(-c3ccc(-c4ccc(-c5ccccc5)c5ccccc45)c4cccnc34)nc(-c3cccc4oc5ccccc5c34)n2)cc1 ; c1ccc(-c2cc(-c3ccccc3)c3nc(-c4nc(-c5ccccc5)nc(-c5cccc6oc7ccccc7c56)n4)ccc3c2)cc1 ; c1ccc(-c2ccc3cc(-c4ccc5nc(-c6nc(-c7ccccc7)nc(-c7ccccc7)n6)ccc5c4)ccc3c2)cc1 ; c1ccc(-c2nc(-c3ccc4c(-c5ccccc5)ccc(-c5ccccc5)c4n3)nc(-c3cccc4oc5ccccc5c34)n2)cc1]

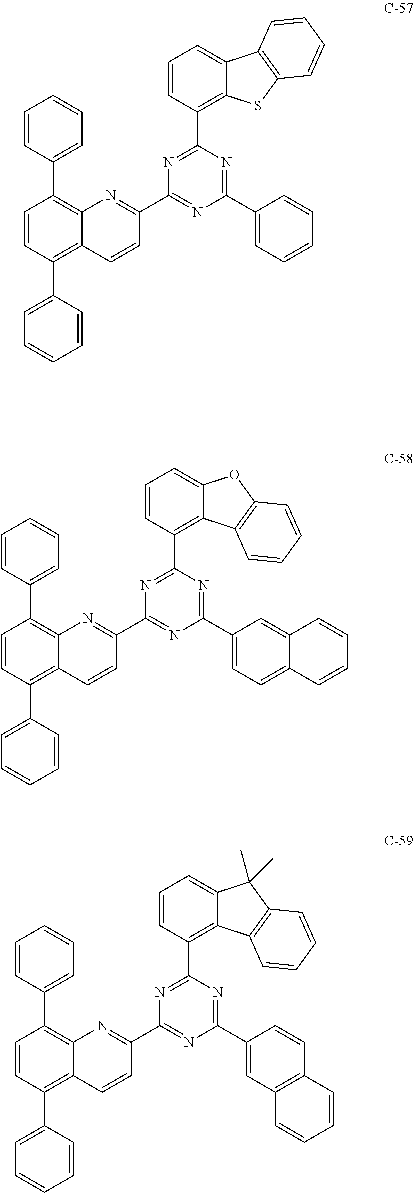 [SMILES: c1ccc(-c2nc(-c3ccc4c(-c5ccccc5)ccc(-c5ccccc5)c4n3)nc(-c3cccc4c3sc3ccccc34)n2)cc1 ; c1ccc(-c2ccc(-c3ccccc3)c3nc(-c4nc(-c5ccc6ccccc6c5)nc(-c5cccc6oc7ccccc7c56)n4)ccc23)cc1 ; CC1(C)c2ccccc2-c2c(-c3nc(-c4ccc5ccccc5c4)nc(-c4ccc5c(-c6ccccc6)ccc(-c6ccccc6)c5n4)n3)cccc21]

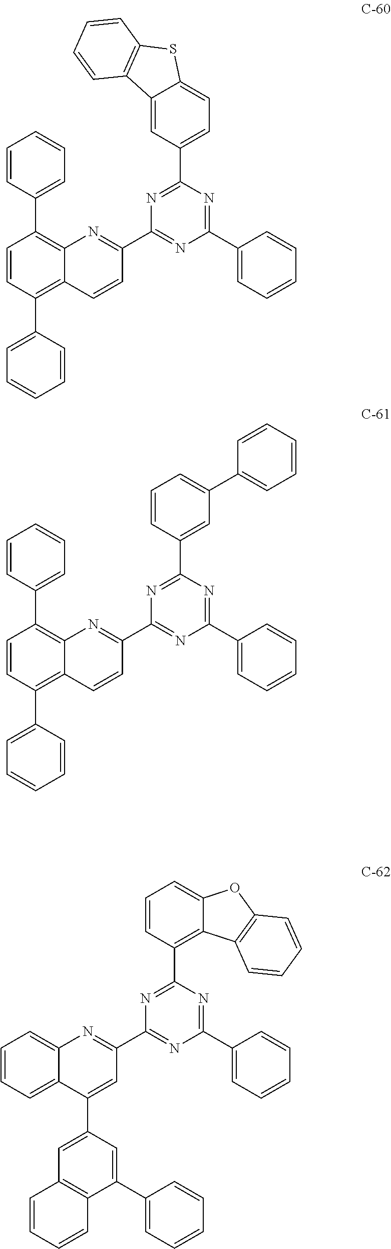 [SMILES: c1ccc(-c2nc(-c3ccc4sc5ccccc5c4c3)nc(-c3ccc4c(-c5ccccc5)ccc(-c5ccccc5)c4n3)n2)cc1 ; c1ccc(-c2cccc(-c3nc(-c4ccccc4)nc(-c4ccc5c(-c6ccccc6)ccc(-c6ccccc6)c5n4)n3)c2)cc1 ; c1ccc(-c2nc(-c3cc(-c4cc(-c5ccccc5)c5ccccc5c4)c4ccccc4n3)nc(-c3cccc4oc5ccccc5c34)n2)cc1]

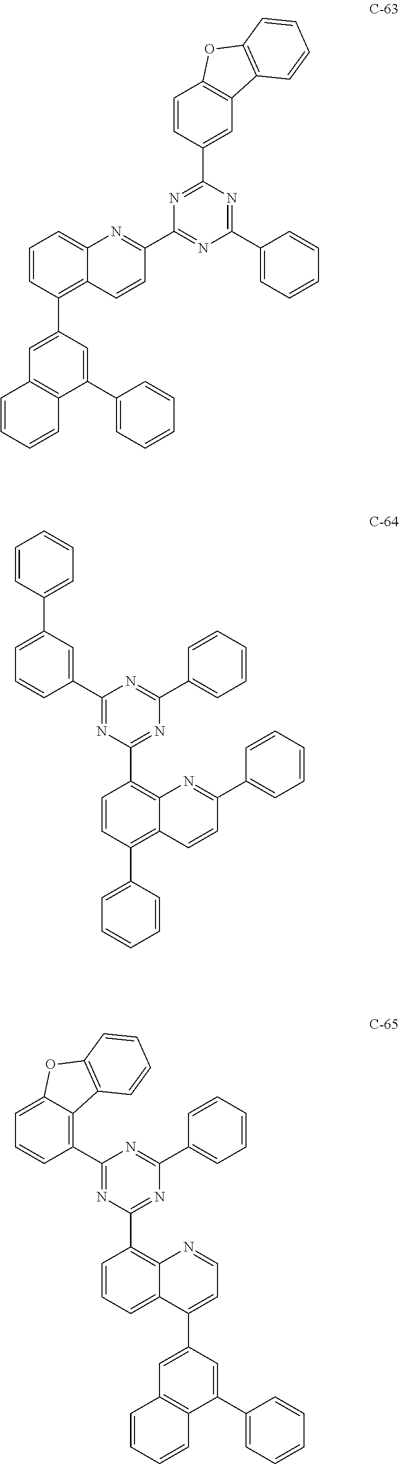 [SMILES: c1ccc(-c2nc(-c3ccc4oc5ccccc5c4c3)nc(-c3ccc4c(-c5cc(-c6ccccc6)c6ccccc6c5)cccc4n3)n2)cc1 ; c1ccc(-c2cccc(-c3nc(-c4ccccc4)nc(-c4ccc(-c5ccccc5)c5ccc(-c6ccccc6)nc45)n3)c2)cc1 ; c1ccc(-c2nc(-c3cccc4c(-c5cc(-c6ccccc6)c6ccccc6c5)ccnc34)nc(-c3cccc4oc5ccccc5c34)n2)cc1]

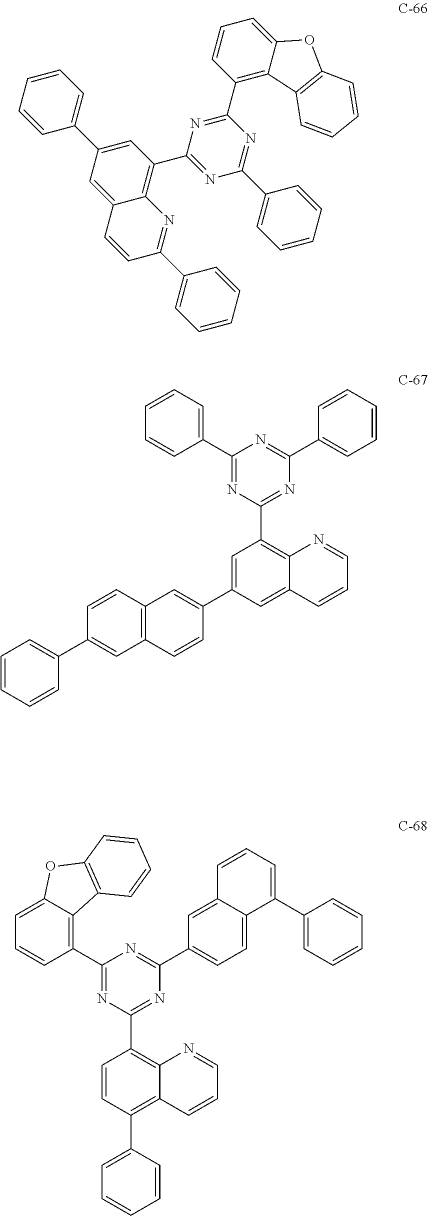 [SMILES: c1ccc(-c2cc(-c3nc(-c4ccccc4)nc(-c4cccc5oc6ccccc6c45)n3)c3nc(-c4ccccc4)ccc3c2)cc1 ; c1ccc(-c2ccc3cc(-c4cc(-c5nc(-c6ccccc6)nc(-c6ccccc6)n5)c5ncccc5c4)ccc3c2)cc1 ; c1ccc(-c2cccc3cc(-c4nc(-c5ccc(-c6ccccc6)c6cccnc56)nc(-c5cccc6oc7ccccc7c56)n4)ccc23)cc1]

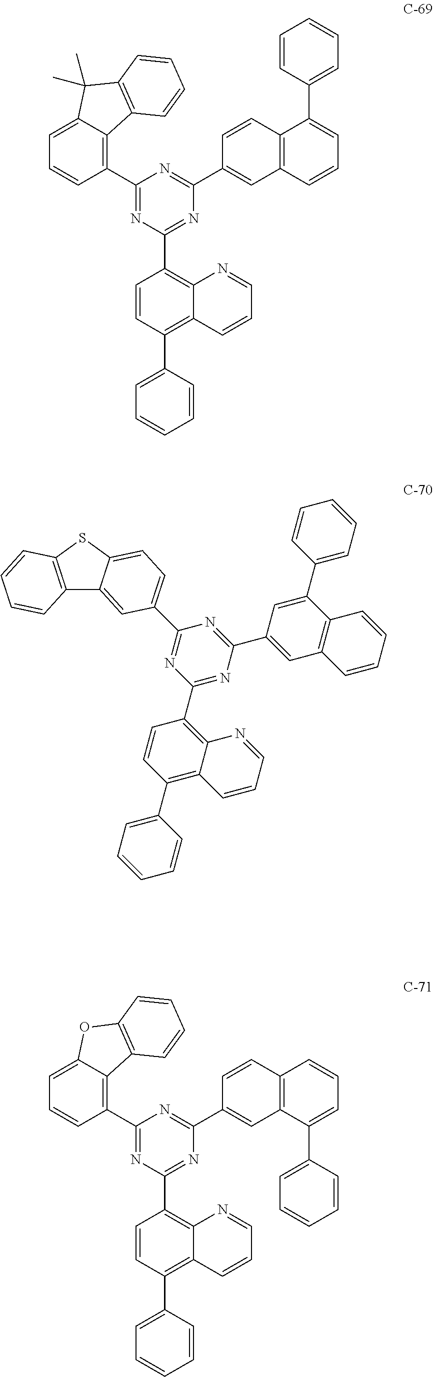 [SMILES: CC1(C)c2ccccc2-c2c(-c3nc(-c4ccc5c(-c6ccccc6)cccc5c4)nc(-c4ccc(-c5ccccc5)c5cccnc45)n3)cccc21 ; c1ccc(-c2cc(-c3nc(-c4ccc5sc6ccccc6c5c4)nc(-c4ccc(-c5ccccc5)c5cccnc45)n3)cc3ccccc23)cc1 ; c1ccc(-c2cccc3ccc(-c4nc(-c5ccc(-c6ccccc6)c6cccnc56)nc(-c5cccc6oc7ccccc7c56)n4)cc23)cc1]

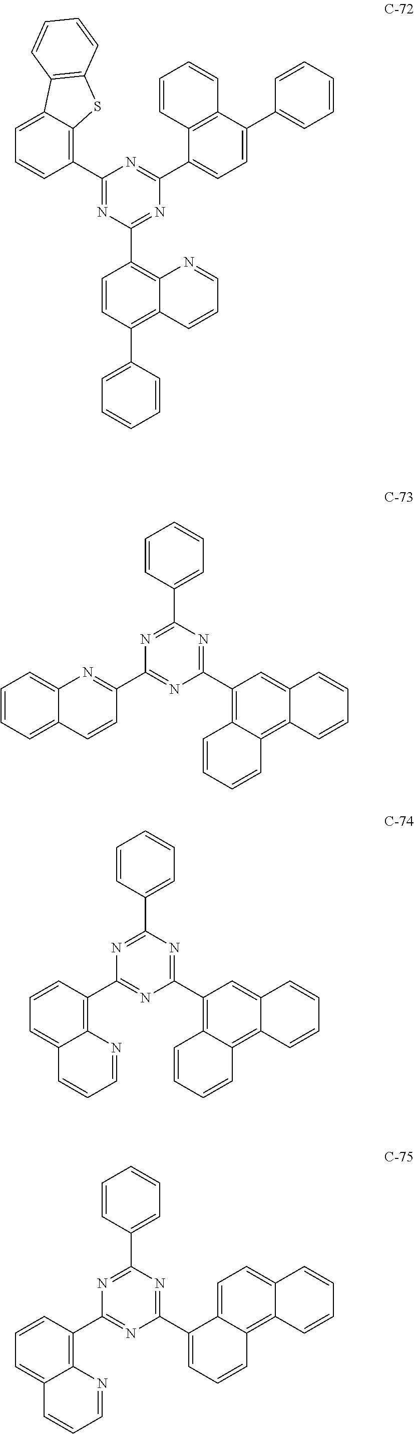 [SMILES: c1ccc(-c2ccc(-c3nc(-c4ccc(-c5ccccc5)c5cccnc45)nc(-c4cccc5c4sc4ccccc45)n3)c3ccccc23)cc1 ; c1ccc(-c2nc(-c3ccc4ccccc4n3)nc(-c3cc4ccccc4c4ccccc34)n2)cc1 ; c1ccc(-c2nc(-c3cc4ccccc4c4ccccc34)nc(-c3cccc4cccnc34)n2)cc1 ; c1ccc(-c2nc(-c3cccc4c3ccc3ccccc34)nc(-c3cccc4cccnc34)n2)cc1]

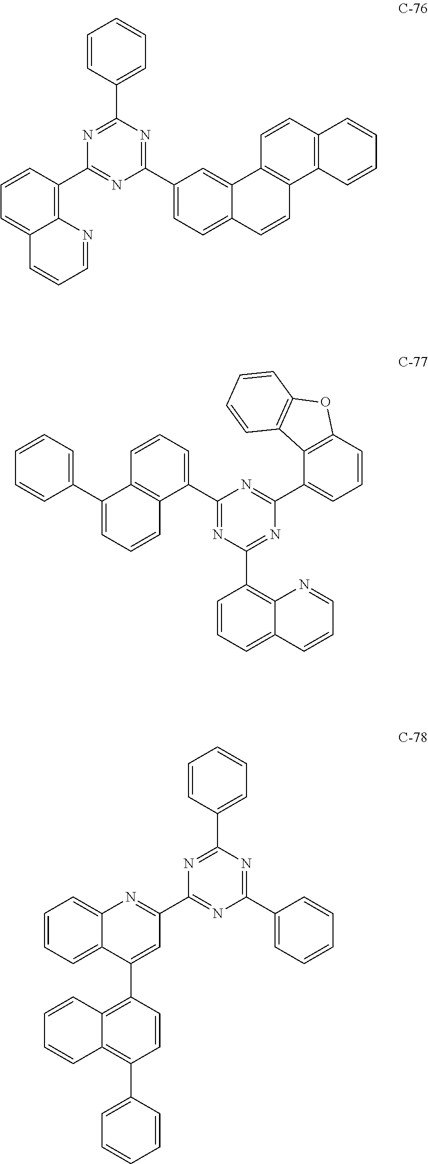 [SMILES: c1ccc(-c2nc(-c3ccc4ccc5c6ccccc6ccc5c4c3)nc(-c3cccc4cccnc34)n2)cc1 ; c1ccc(-c2cccc3c(-c4nc(-c5cccc6cccnc56)nc(-c5cccc6oc7ccccc7c56)n4)cccc23)cc1 ; c1ccc(-c2nc(-c3ccccc3)nc(-c3cc(-c4ccc(-c5ccccc5)c5ccccc45)c4ccccc4n3)n2)cc1]

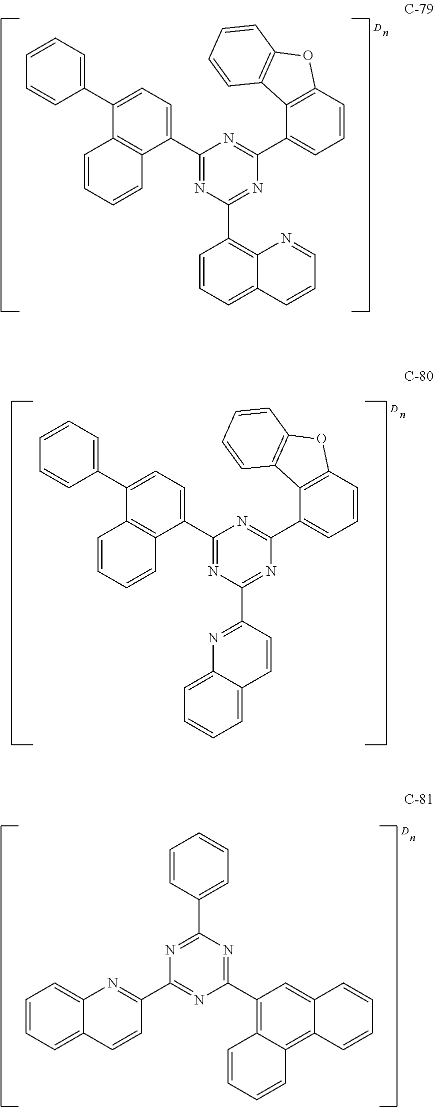

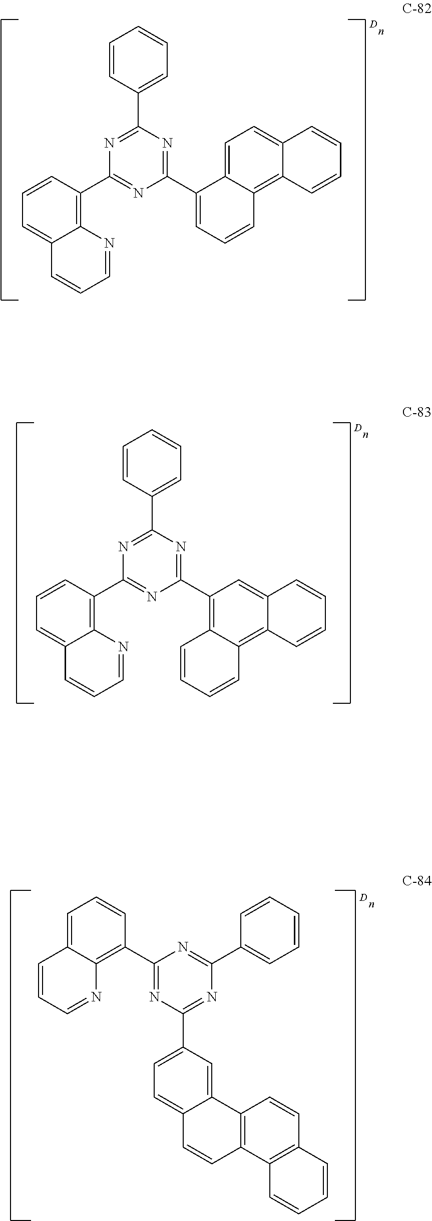

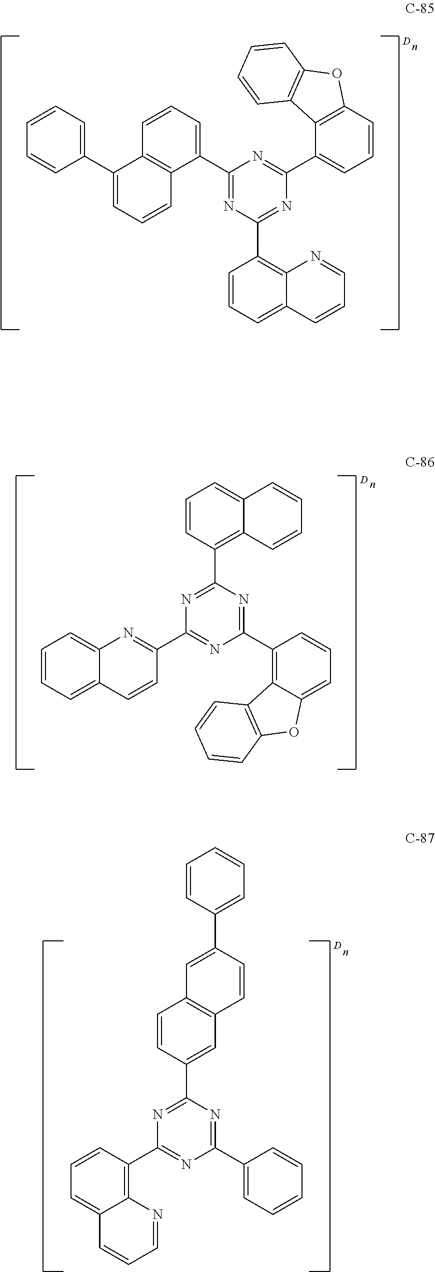

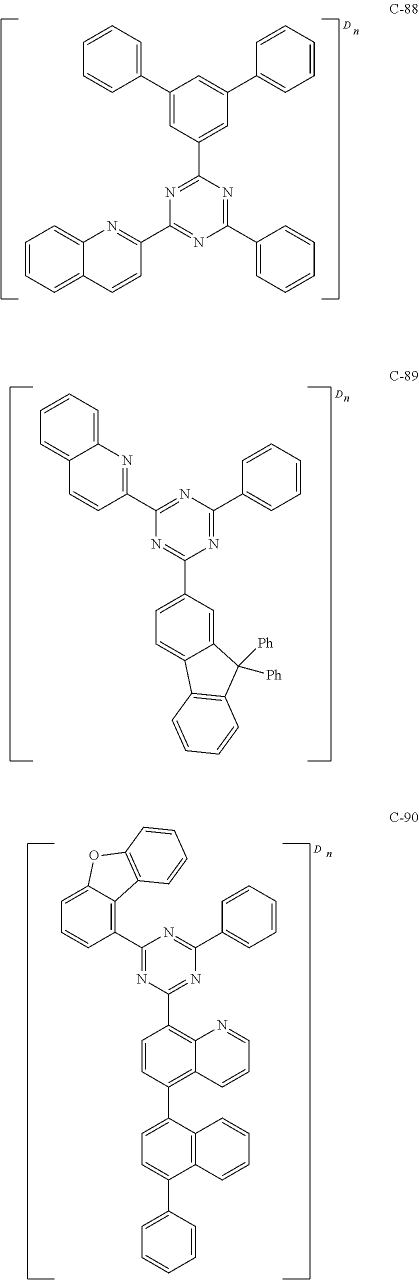

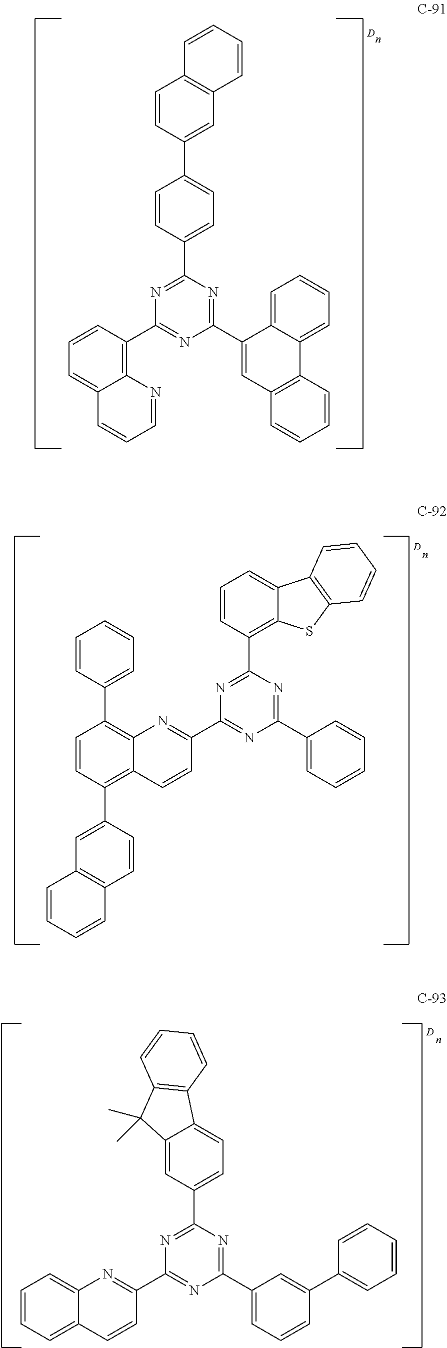

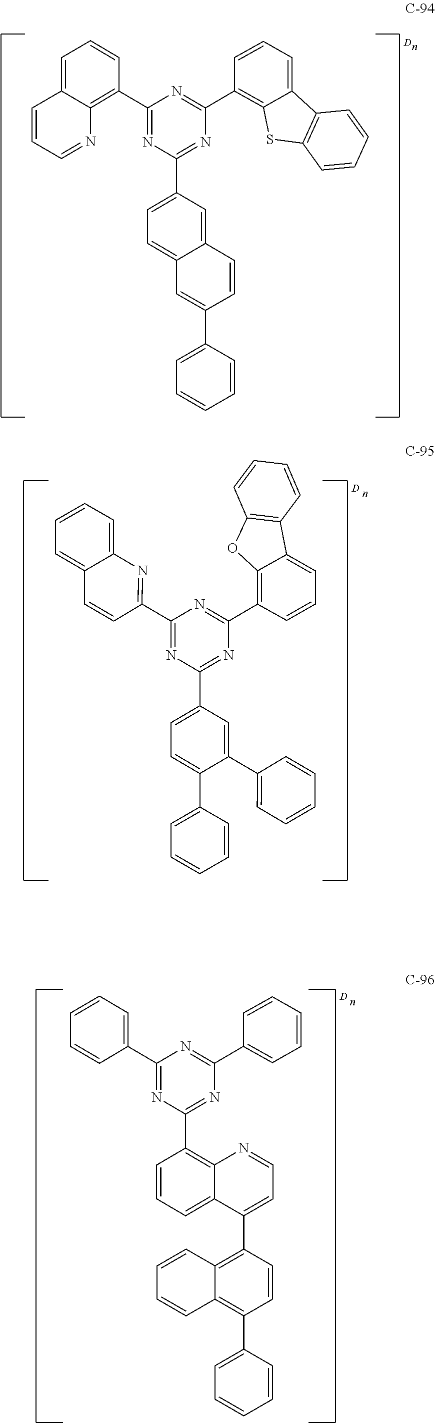

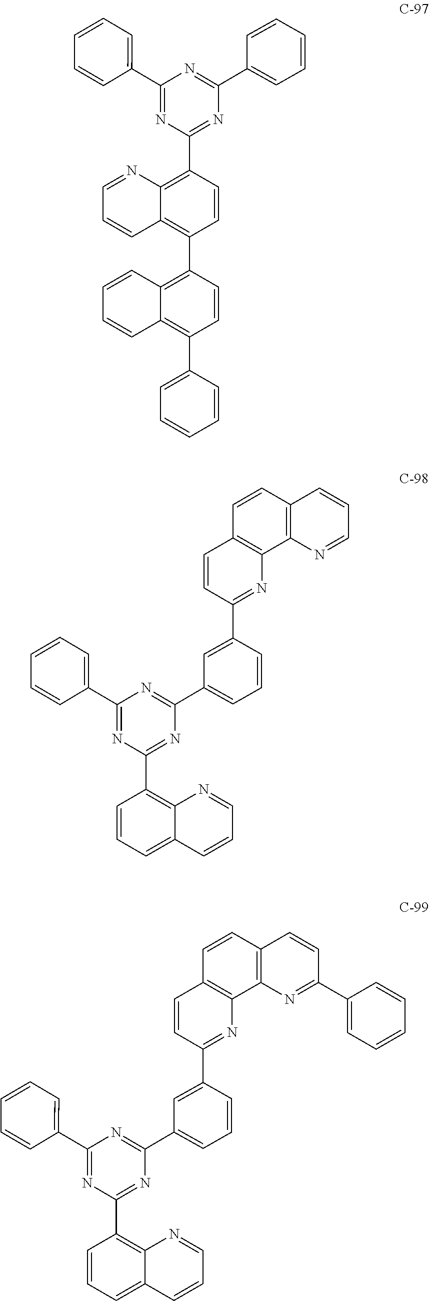 [SMILES: c1ccc(-c2nc(-c3ccccc3)nc(-c3ccc(-c4ccc(-c5ccccc5)c5ccccc45)c4cccnc34)n2)cc1 ; c1ccc(-c2nc(-c3cccc(-c4ccc5ccc6cccnc6c5n4)c3)nc(-c3cccc4cccnc34)n2)cc1 ; c1ccc(-c2ccc3ccc4ccc(-c5cccc(-c6nc(-c7ccccc7)nc(-c7cccc8cccnc78)n6)c5)nc4c3n2)cc1]

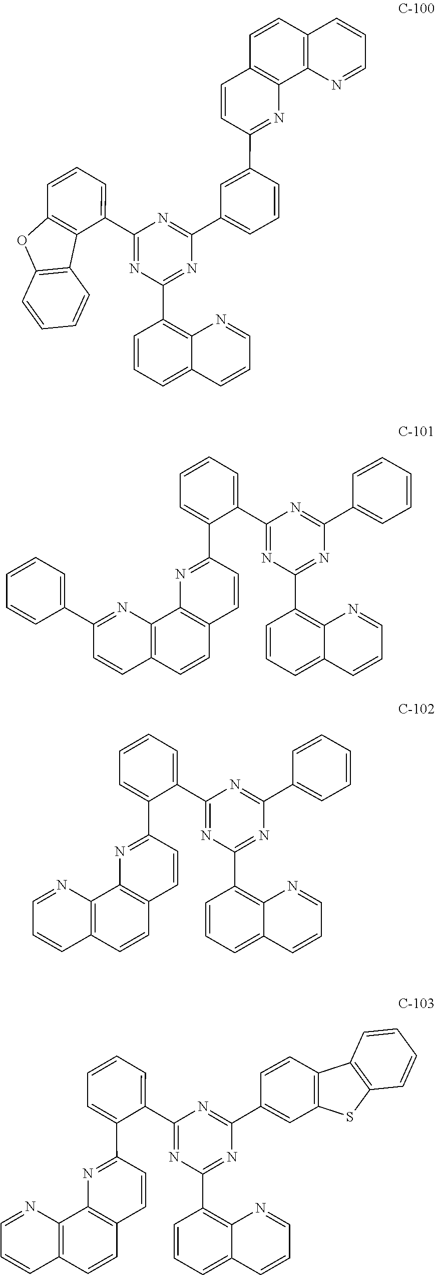 [SMILES: c1cc(-c2ccc3ccc4cccnc4c3n2)cc(-c2nc(-c3cccc4cccnc34)nc(-c3cccc4oc5ccccc5c34)n2)c1 ; c1ccc(-c2ccc3ccc4ccc(-c5ccccc5-c5nc(-c6ccccc6)nc(-c6cccc7cccnc67)n5)nc4c3n2)cc1 ; c1ccc(-c2nc(-c3ccccc3-c3ccc4ccc5cccnc5c4n3)nc(-c3cccc4cccnc34)n2)cc1 ; c1ccc(-c2nc(-c3ccc4c(c3)sc3ccccc34)nc(-c3cccc4cccnc34)n2)c(-c2ccc3ccc4cccnc4c3n2)c1]

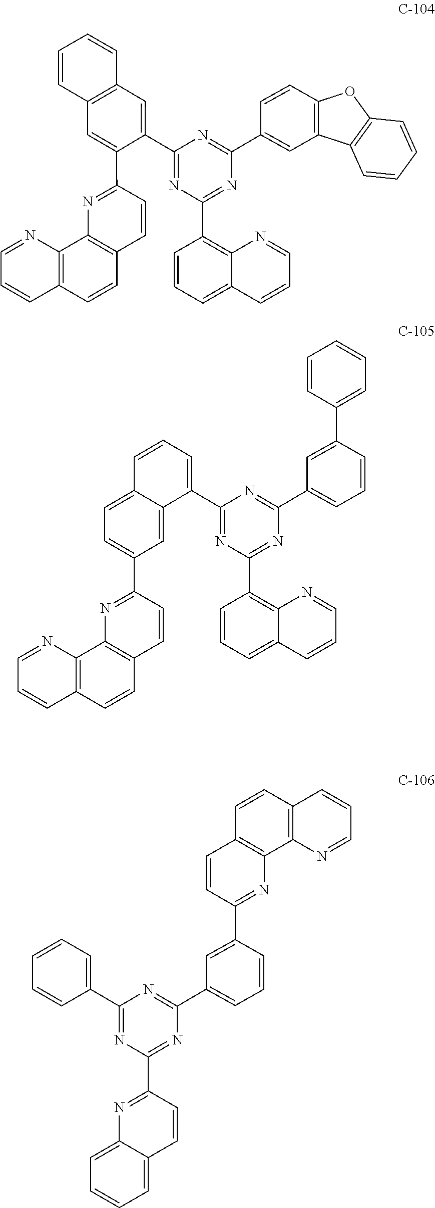 [SMILES: c1ccc2cc(-c3nc(-c4ccc5oc6ccccc6c5c4)nc(-c4cccc5cccnc45)n3)c(-c3ccc4ccc5cccnc5c4n3)cc2c1 ; c1ccc(-c2cccc(-c3nc(-c4cccc5ccc(-c6ccc7ccc8cccnc8c7n6)cc45)nc(-c4cccc5cccnc45)n3)c2)cc1 ; c1ccc(-c2nc(-c3cccc(-c4ccc5ccc6cccnc6c5n4)c3)nc(-c3ccc4ccccc4n3)n2)cc1]

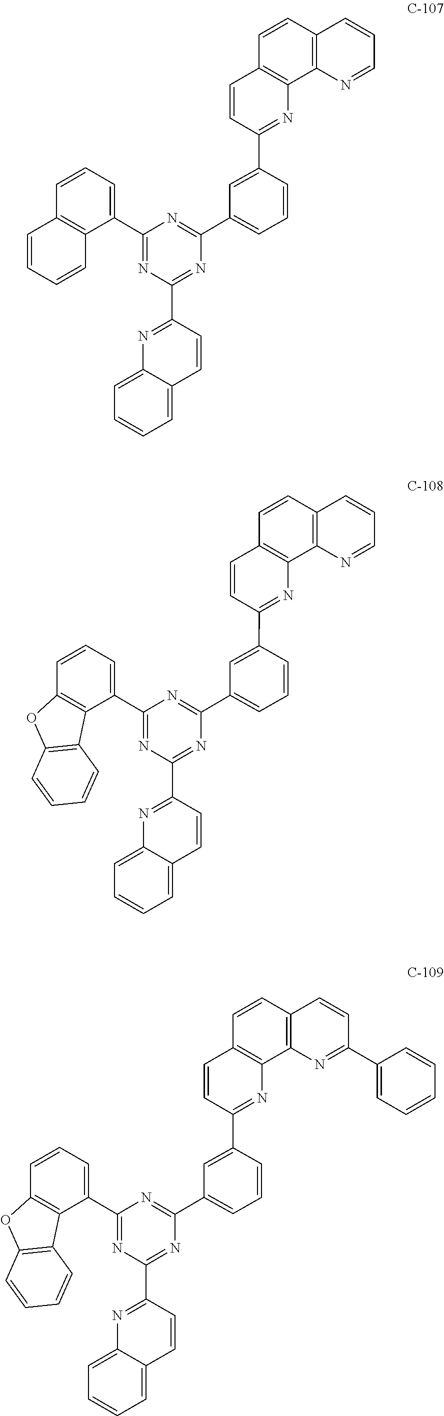 [SMILES: c1cc(-c2ccc3ccc4cccnc4c3n2)cc(-c2nc(-c3ccc4ccccc4n3)nc(-c3cccc4ccccc34)n2)c1 ; c1cc(-c2ccc3ccc4cccnc4c3n2)cc(-c2nc(-c3ccc4ccccc4n3)nc(-c3cccc4oc5ccccc5c34)n2)c1 ; c1ccc(-c2ccc3ccc4ccc(-c5cccc(-c6nc(-c7ccc8ccccc8n7)nc(-c7cccc8oc9ccccc9c78)n6)c5)nc4c3n2)cc1]

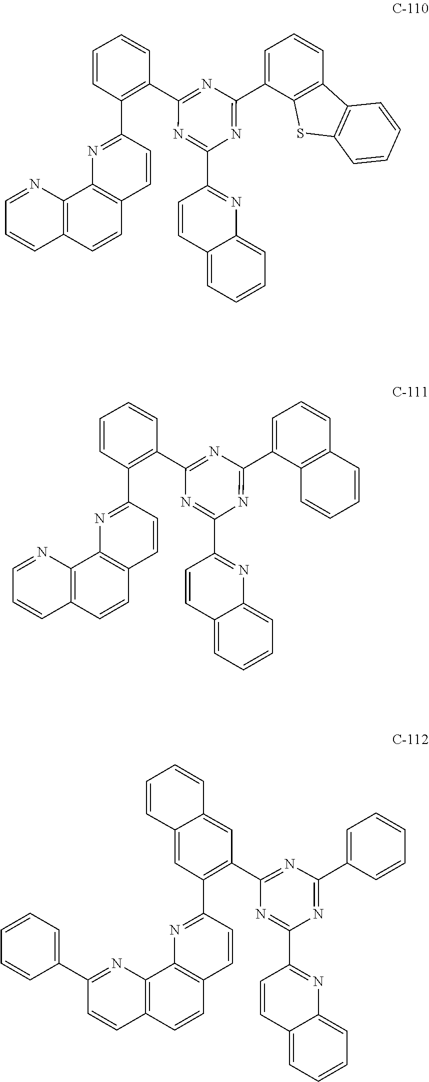 [SMILES: c1ccc(-c2nc(-c3ccc4ccccc4n3)nc(-c3cccc4c3sc3ccccc34)n2)c(-c2ccc3ccc4cccnc4c3n2)c1 ; c1ccc(-c2nc(-c3ccc4ccccc4n3)nc(-c3cccc4ccccc34)n2)c(-c2ccc3ccc4cccnc4c3n2)c1 ; c1ccc(-c2ccc3ccc4ccc(-c5cc6ccccc6cc5-c5nc(-c6ccccc6)nc(-c6ccc7ccccc7n6)n5)nc4c3n2)cc1]

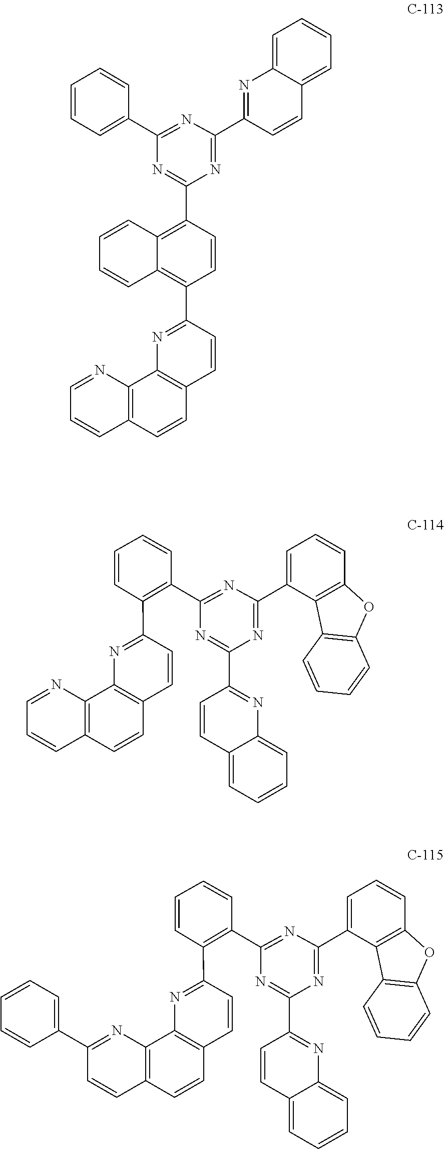 [SMILES: c1ccc(-c2nc(-c3ccc4ccccc4n3)nc(-c3ccc(-c4ccc5ccc6cccnc6c5n4)c4ccccc34)n2)cc1 ; c1ccc(-c2nc(-c3ccc4ccccc4n3)nc(-c3cccc4oc5ccccc5c34)n2)c(-c2ccc3ccc4cccnc4c3n2)c1 ; c1ccc(-c2ccc3ccc4ccc(-c5ccccc5-c5nc(-c6ccc7ccccc7n6)nc(-c6cccc7oc8ccccc8c67)n5)nc4c3n2)cc1]

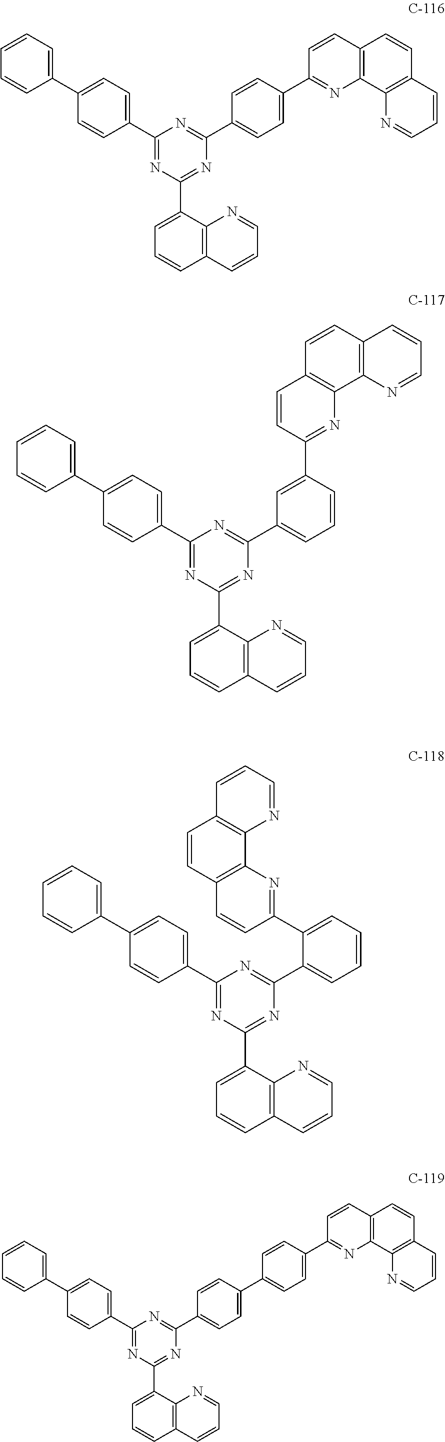 [SMILES: c1ccc(-c2ccc(-c3nc(-c4ccc(-c5ccc6ccc7cccnc7c6n5)cc4)nc(-c4cccc5cccnc45)n3)cc2)cc1 ; c1ccc(-c2ccc(-c3nc(-c4cccc(-c5ccc6ccc7cccnc7c6n5)c4)nc(-c4cccc5cccnc45)n3)cc2)cc1 ; c1ccc(-c2ccc(-c3nc(-c4ccccc4-c4ccc5ccc6cccnc6c5n4)nc(-c4cccc5cccnc45)n3)cc2)cc1 ; c1ccc(-c2ccc(-c3nc(-c4ccc(-c5ccc(-c6ccc7ccc8cccnc8c7n6)cc5)cc4)nc(-c4cccc5cccnc45)n3)cc2)cc1]

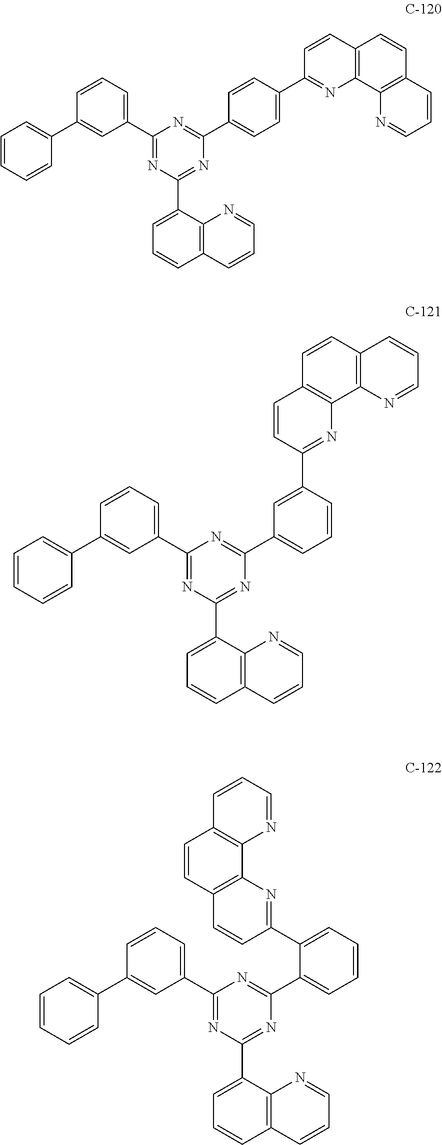 [SMILES: c1ccc(-c2cccc(-c3nc(-c4ccc(-c5ccc6ccc7cccnc7c6n5)cc4)nc(-c4cccc5cccnc45)n3)c2)cc1 ; c1ccc(-c2cccc(-c3nc(-c4cccc(-c5ccc6ccc7cccnc7c6n5)c4)nc(-c4cccc5cccnc45)n3)c2)cc1 ; c1ccc(-c2cccc(-c3nc(-c4ccccc4-c4ccc5ccc6cccnc6c5n4)nc(-c4cccc5cccnc45)n3)c2)cc1]

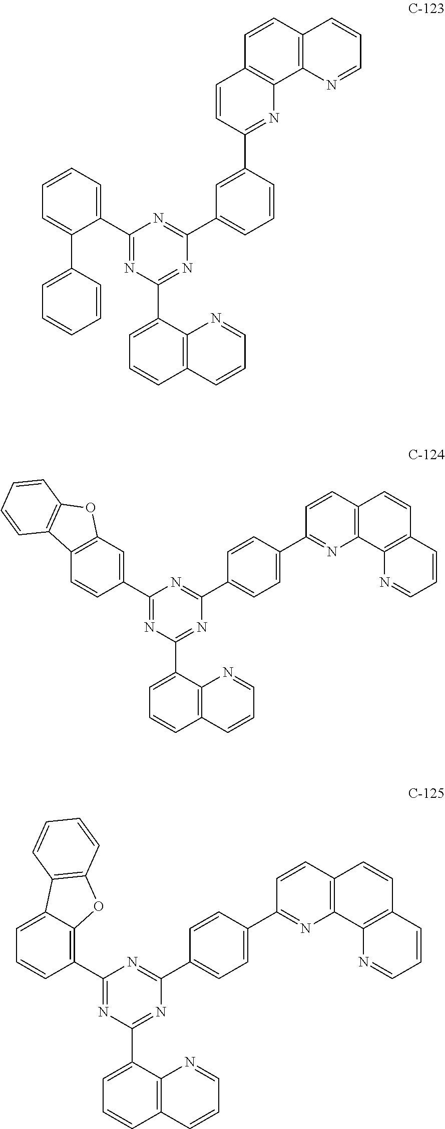 [SMILES: c1ccc(-c2ccccc2-c2nc(-c3cccc(-c4ccc5ccc6cccnc6c5n4)c3)nc(-c3cccc4cccnc34)n2)cc1 ; c1cnc2c(-c3nc(-c4ccc(-c5ccc6ccc7cccnc7c6n5)cc4)nc(-c4ccc5c(c4)oc4ccccc45)n3)cccc2c1 ; c1cnc2c(-c3nc(-c4ccc(-c5ccc6ccc7cccnc7c6n5)cc4)nc(-c4cccc5c4oc4ccccc45)n3)cccc2c1]

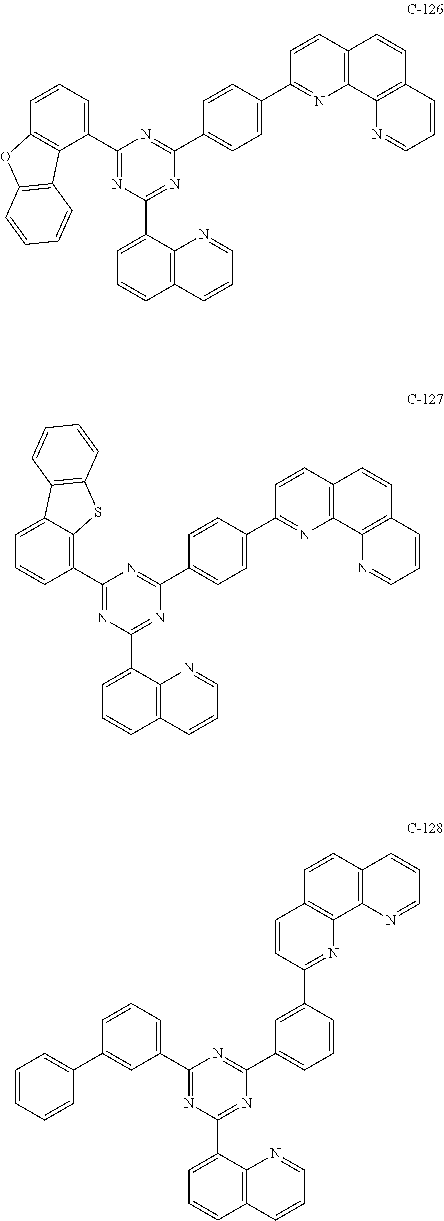 [SMILES: c1cnc2c(-c3nc(-c4ccc(-c5ccc6ccc7cccnc7c6n5)cc4)nc(-c4cccc5oc6ccccc6c45)n3)cccc2c1 ; c1cnc2c(-c3nc(-c4ccc(-c5ccc6ccc7cccnc7c6n5)cc4)nc(-c4cccc5c4sc4ccccc45)n3)cccc2c1 ; c1ccc(-c2cccc(-c3nc(-c4cccc(-c5ccc6ccc7cccnc7c6n5)c4)nc(-c4cccc5cccnc45)n3)c2)cc1]

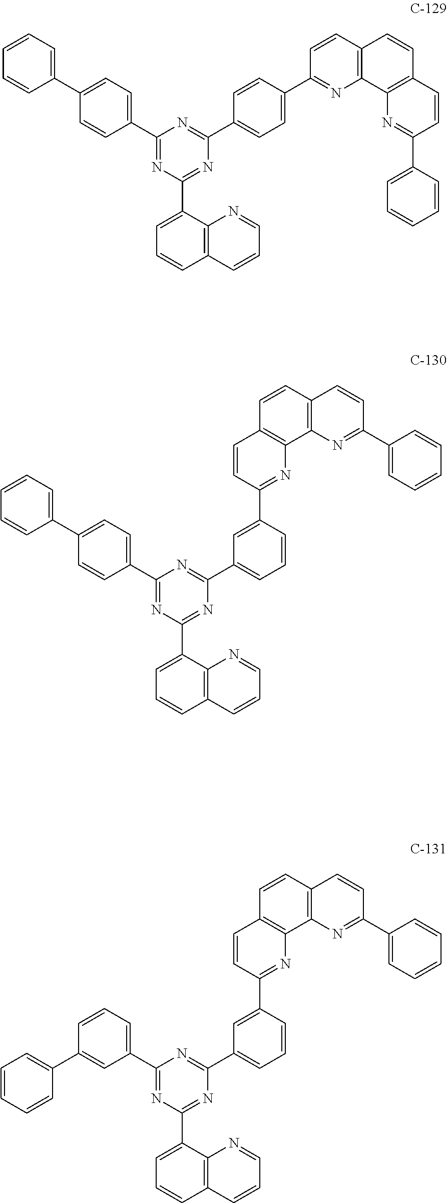 [SMILES: c1ccc(-c2ccc(-c3nc(-c4ccc(-c5ccc6ccc7ccc(-c8ccccc8)nc7c6n5)cc4)nc(-c4cccc5cccnc45)n3)cc2)cc1 ; c1ccc(-c2ccc(-c3nc(-c4cccc(-c5ccc6ccc7ccc(-c8ccccc8)nc7c6n5)c4)nc(-c4cccc5cccnc45)n3)cc2)cc1 ; c1ccc(-c2cccc(-c3nc(-c4cccc(-c5ccc6ccc7ccc(-c8ccccc8)nc7c6n5)c4)nc(-c4cccc5cccnc45)n3)c2)cc1]

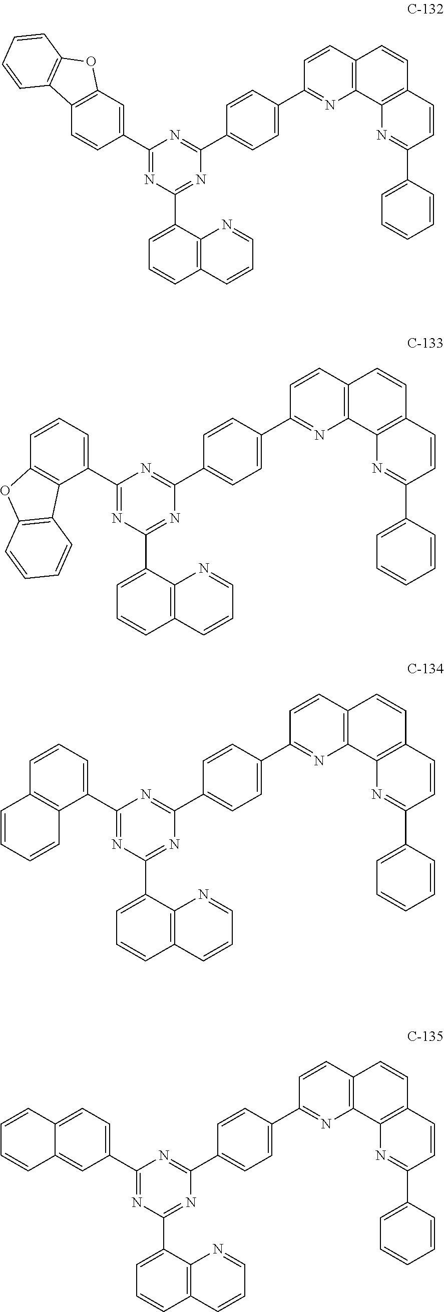 [SMILES: c1ccc(-c2ccc3ccc4ccc(-c5ccc(-c6nc(-c7ccc8c(c7)oc7ccccc78)nc(-c7cccc8cccnc78)n6)cc5)nc4c3n2)cc1 ; c1ccc(-c2ccc3ccc4ccc(-c5ccc(-c6nc(-c7cccc8cccnc78)nc(-c7cccc8oc9ccccc9c78)n6)cc5)nc4c3n2)cc1 ; c1ccc(-c2ccc3ccc4ccc(-c5ccc(-c6nc(-c7cccc8ccccc78)nc(-c7cccc8cccnc78)n6)cc5)nc4c3n2)cc1 ; c1ccc(-c2ccc3ccc4ccc(-c5ccc(-c6nc(-c7ccc8ccccc8c7)nc(-c7cccc8cccnc78)n6)cc5)nc4c3n2)cc1]

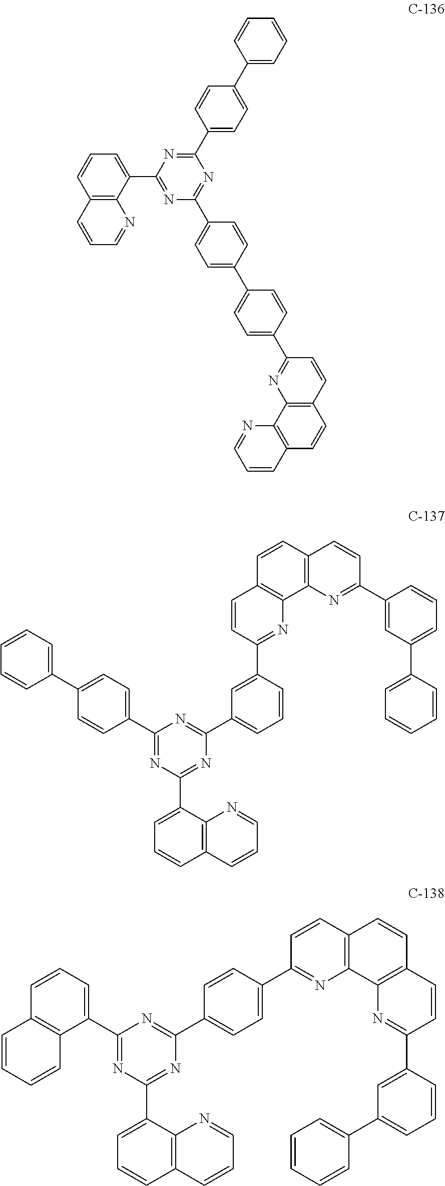 [SMILES: c1ccc(-c2ccc(-c3nc(-c4ccc(-c5ccc(-c6ccc7ccc8cccnc8c7n6)cc5)cc4)nc(-c4cccc5cccnc45)n3)cc2)cc1 ; c1ccc(-c2ccc(-c3nc(-c4cccc(-c5ccc6ccc7ccc(-c8cccc(-c9ccccc9)c8)nc7c6n5)c4)nc(-c4cccc5cccnc45)n3)cc2)cc1 ; c1ccc(-c2cccc(-c3ccc4ccc5ccc(-c6ccc(-c7nc(-c8cccc9ccccc89)nc(-c8cccc9cccnc89)n7)cc6)nc5c4n3)c2)cc1]

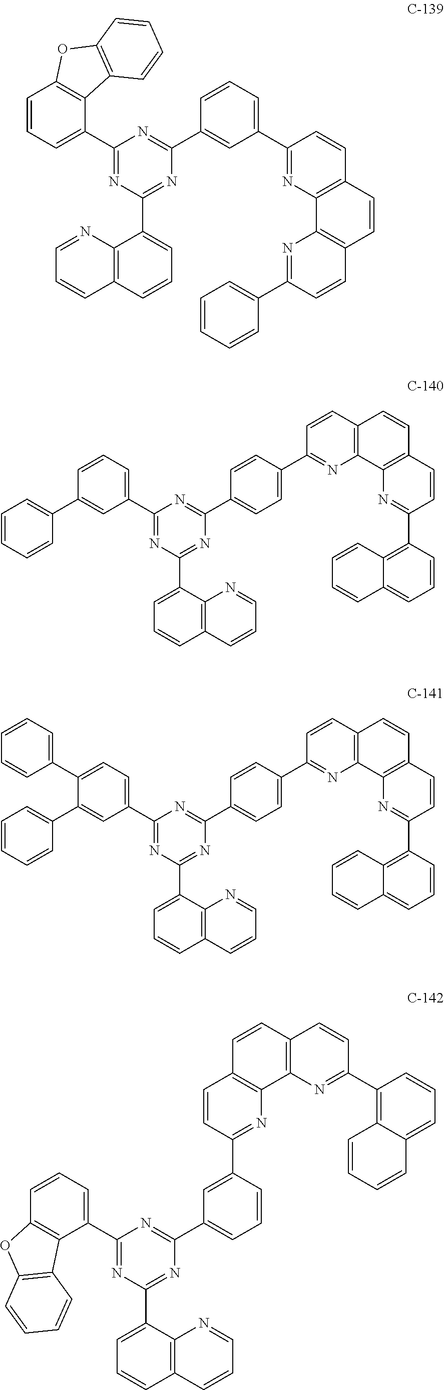 [SMILES: c1ccc(-c2ccc3ccc4ccc(-c5cccc(-c6nc(-c7cccc8cccnc78)nc(-c7cccc8oc9ccccc9c78)n6)c5)nc4c3n2)cc1 ; c1ccc(-c2cccc(-c3nc(-c4ccc(-c5ccc6ccc7ccc(-c8cccc9ccccc89)nc7c6n5)cc4)nc(-c4cccc5cccnc45)n3)c2)cc1 ; c1ccc(-c2ccc(-c3nc(-c4ccc(-c5ccc6ccc7ccc(-c8cccc9ccccc89)nc7c6n5)cc4)nc(-c4cccc5cccnc45)n3)cc2-c2ccccc2)cc1 ; c1cc(-c2ccc3ccc4ccc(-c5cccc6ccccc56)nc4c3n2)cc(-c2nc(-c3cccc4cccnc34)nc(-c3cccc4oc5ccccc5c34)n2)c1]

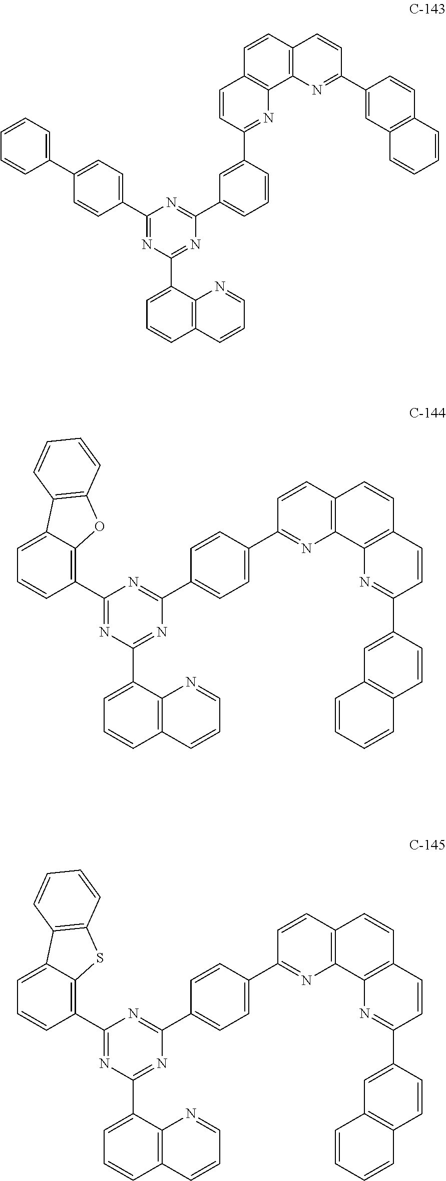 [SMILES: c1ccc(-c2ccc(-c3nc(-c4cccc(-c5ccc6ccc7ccc(-c8ccc9ccccc9c8)nc7c6n5)c4)nc(-c4cccc5cccnc45)n3)cc2)cc1 ; c1ccc2cc(-c3ccc4ccc5ccc(-c6ccc(-c7nc(-c8cccc9cccnc89)nc(-c8cccc9c8oc8ccccc89)n7)cc6)nc5c4n3)ccc2c1 ; c1ccc2cc(-c3ccc4ccc5ccc(-c6ccc(-c7nc(-c8cccc9cccnc89)nc(-c8cccc9c8sc8ccccc89)n7)cc6)nc5c4n3)ccc2c1]

In compounds C-79 to C-96, “Dₙ” means that n number of hydrogens is replaced by deuterium, wherein n is from 1 to the maximum number of hydrogen in the compound.

The triazinyl quinoline derivative compound represented by formula 1 according to the present disclosure may be prepared as shown in Reaction Schemes 1 and 2 below, but is not limited thereto, and may also be prepared by synthetic methods known to a person skilled in the art.

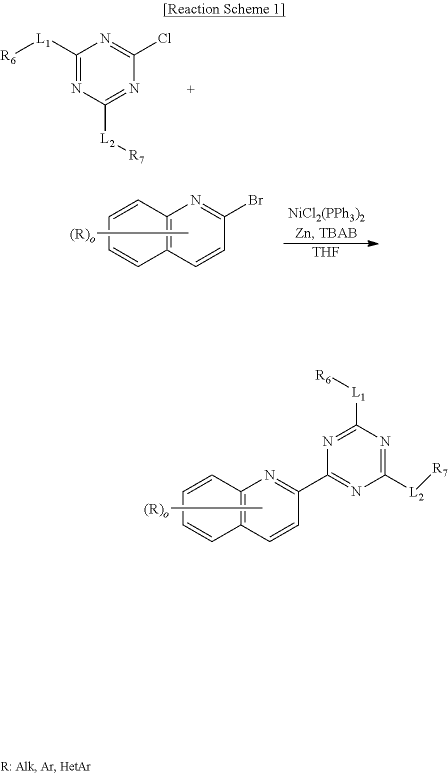

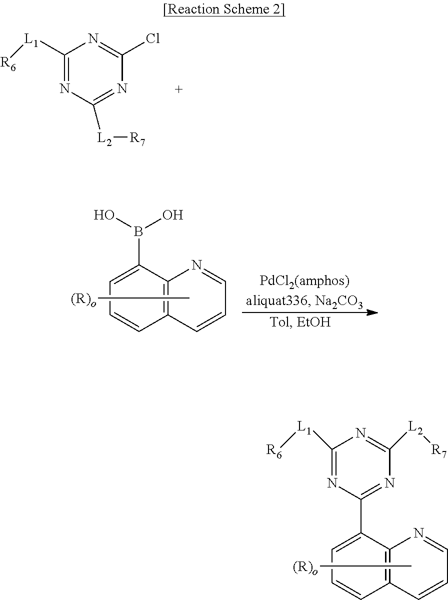

In Reaction Schemes 1 and 2, the definition of R is the same as the definitions of R₁ to R₅ of formula 1, o is an integer of 0 to 6, and the definitions of R₆, R₇, L₁, and L₂ are the same as definitions in formula 1.

Although illustrative synthesis examples of the compound represented by formula 1 are described above, a person skilled in the art will be able to readily understand that all of them are based on a Buchwald-Hartwig cross coupling reaction, an N-arylation reaction, a H-mont-mediated etherification reaction, a Miyaura borylation reaction, a Suzuki cross-coupling reaction, a Wittig reaction, an intramolecular acid-induced cyclization reaction, a Pd(II)-catalyzed oxidative cyclization reaction, a Grignard reaction, a Heck reaction, a cyclic dehydration reaction, a SN1 substitution reaction, a SN2 substitution reaction, a phosphine-mediated reductive cyclization reaction, etc. It will be readily understood by a person skilled in the art that the above reactions proceed even if other substituents defined in formula 1, other than the substituents described in the specific synthesis examples, are bonded.

According to another embodiment, the present disclosure provides an organic electroluminescent device, wherein an organic electroluminescent material comprising a compound represented by formula 1 is applied.

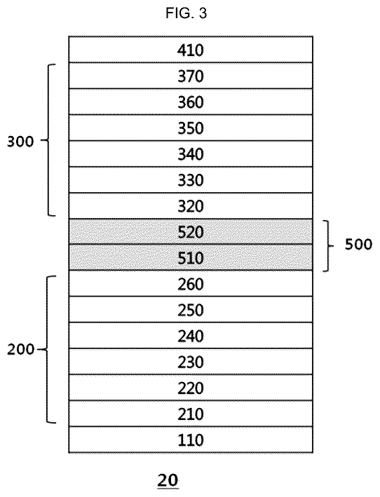

FIGS. 2 and 3 each illustrate an example of an organic electroluminescent device according to one embodiment.

Herein, “first” and “second” and the like are added for convenience to refer to each layers contained within a plurality of light-emitting units. For explaining common functionalities, the expressions such as “first” and “second” and the like may be omitted.

Hereinafter, an organic electroluminescent device applying the aforementioned organic electroluminescent material will be described with reference to the drawings.

According to one embodiment, an organic electroluminescence device comprising a triazinyl quinoline derivative compound is provided. Specifically, the organic electroluminescence device according to one embodiment of the present disclosure comprises: a plurality of light-emitting units positioned between a first electrode and a second electrode and comprising at least one light-emitting layer(s); and at least one n-type charge generation layer(s) positioned between the adjacent light-emitting units, wherein the n-type charge generation layer includes a triazinyl quinoline derivative compound. The n-type charge generation layer may be additionally doped with a metal, and the triazinyl quinoline derivative compound according to the present disclosure may form a complex to fix the metal. In addition, the triazinyl quinoline derivative compound of the present disclosure may have a bidentate structure to effectively fix the metal.

As shown in FIG. 2, an organic electroluminescent device 10 according to one embodiment includes a first electrode 110 and a second electrode 410 opposite the first electrode 110, and a plurality of light-emitting units 200, 300 positioned between the first electrode 110 and the second electrode 410, and at least one charge generation layer 500 positioned between adjacent light-emitting units 200, 300. The light-emitting units 200, 300 include at least one light-emitting layer 240, 340, and the charge generation layer 500 includes an n-type charge generation layer 510 and a p-type charge generation layer 520. In this case, the n-type charge generation layer 510 comprises a compound represented by formula 1.

The light-emitting units included in the organic electroluminescent device according to one embodiment are at least two, and the charge generation layer can be positioned between adjacent light-emitting units, allowing for the addition of more light-emitting units.

According to one embodiment, each light-emitting unit 200, 300 includes hole transport layers 220, 320, light-emitting layers 240, 340, and electron transport layers 260, 360. Specifically, the first light-emitting unit 200 may include a first hole transport layer 220, a first light-emitting layer 240, and a first electron transport layer 260, and the second light-emitting unit 300 may include a second hole transport layer 320, a second light-emitting layer 340, and a second electron transport layer 360. Further, the first light-emitting unit 200 may further include a hole injection layer 210, and the second light-emitting unit 300 may further include an electron injection layer 370.

The light-emitting layers 240, 340 are light-emitting layers comprising a host and a dopant, which may be a single layer or a plurality of layers with two or more layers stacked. In this case, the host mainly has the function of promoting the recombination of electrons and holes and trapping excitons in the light-emitting layer, and the dopant has the function of efficiently emitting excitons obtained by recombination. The dopant compounds in the light-emitting layers 240, 340 may be doped at less than 25 wt %, preferably less than 17 wt %, more preferably less than 10%, with respect to the host compound and the dopant compounds as a whole.

According to one embodiment, the charge generation layer 500 includes an n-type charge generation layer 510 located adjacent to the first light-emitting unit 200 to supply electrons to the first light-emitting unit 200 and a p-type charge generation layer 520 located adjacent to the second light-emitting unit 300 to supply holes to the second light-emitting unit 300.

The n-type charge generation layer 510 comprises a compound represented by formula 1. The compound represented by formula 1 has a deep energy level and at the same time has an excellent binding ability to metals. Therefore, when the compound represented by formula 1 above is applied to an organic electroluminescent device as an n-type charge generation layer material, the amount of electron injection into the light-emitting unit can be improved by rapidly receiving electrons from the p-type charge generation layer, and the phenomenon of diffusion of the alkali metal doped in the n-type charge generation layer 510 into the p-type charge generation layer 520 can be minimized.

According to one embodiment, the n-type charge generation layer 510 may further include an n-type dopant to enhance the electron injection characteristics into the n-type charge generation layer. For example, usable n-type dopants may further comprise alkali metals such as Li, Na, K, Rb, Cs, Fr, and the like, alkaline earth metals such as Be, Mg, Ca, Sr, Ba, Ra, and the like, or one or more complex compounds comprising such metals, as is common in the art. In the n-type charge generation layer 510, the doping concentration of the dopant may be from 0.5% to 10% of the compound of formula 1.

The p-type charge generation layer 520 may also be used as a hole injection layer, and may include hole injection layer material alone, or may include a mixture of hole injection layer material and hole transport material together.

The present disclosure may include deuterated compounds in at least one layer of the light-emitting layers 240, 340 and n-type charge generation layer 510. Specifically, the at least one n-type charge generation layer 510 included in the light-emitting units 200, 300 comprises a deuterated compound represented by formula 1.

One of the first electrode 110 and second electrode 410 may be an anode and the other may be a cathode. In this case, the first electrode 110 and the second electrode 410 may be formed of a transparent conductive material or formed of a semi-transparent or reflective conductive material, respectively. Depending on the type of material forming the first electrode 110 and the second electrode 410, the organic electroluminescent device may be a front-emitting type, a back-emitting type, or a double-emitting type.

Referring to FIG. 3, the light-emitting units 200, 300 according to one embodiment may further include the hole blocking layers 250, 350 between the light-emitting layers 240, 340 and the electron transport layers 260, 360.

The hole blocking layers 250, 350 are layers that prevent holes from reaching the cathode, which can improve the probability of recombination of electrons and holes in the light-emitting layer. A plurality of layers may be used for the hole blocking layers 250, 350 or the electron transport layers 260, 360, and a plurality of compounds may be used for each layer. Further, the electron injection layer 370 may be doped with n-dopant.

The hole injection layer 210 may have a plurality of layers for the purpose of lowering the hole injection barrier (or hole injection voltage) from the anode to the hole transport layers 220, 230 or an electronic blocking layer, and each layer may have two compounds used simultaneously. Additionally, the hole injection layer 210 may be doped with p-dopant. Although not shown, an electron blocking layer may be positioned between the hole transport layer (or hole injection layer) and the light-emitting layers 240, 340 to block the overflow of electrons from the light-emitting layer, thereby trapping excitons within the light-emitting layer and preventing light emission leakage. A plurality of layers may be used for the hole transport layers 220, 230, 320, 330 or the electron blocking layer, and a plurality of compounds may be used for each layer. If the organic electroluminescent device includes more than two layers of hole transport layers, the additional hole transport layers may be used as hole auxiliary layers or electron blocking layers.

The organic electroluminescent device according to one embodiment may comprise a light-emitting auxiliary layer positioned between the anode and the light-emitting layer or between the cathode and the light-emitting layer. When the light-emitting auxiliary layer is located between the anode and the light-emitting layer, it may be used to facilitate the injection and/or transfer of holes or to block the overflow of electrons, and when the light-emitting auxiliary layer is located between the cathode and the light-emitting layer, it may be used to facilitate the injection and/or transfer of electrons or to block the overflow of holes. In addition, the organic electroluminescent device according to one embodiment may further comprise a hole auxiliary layer located between the hole transport layer (or hole injection layer) and the light-emitting layer. The hole auxiliary layer may have the effect of smoothing or blocking the hole transfer rate (or injection rate), thereby adjusting the charge balance.

In the organic electroluminescent device of the present disclosure, preferably, at least one inner surface layer(s) selected from a chalcogenide layer, a metal halide layer, and a metal oxide layer (hereinafter referred to as “surface layers”) may be placed on an inner surface(s) of at least one of the pair of electrodes. Specifically, a chalcogenide (including oxides) layer of silicon and aluminum is preferably placed on an anode surface of an electroluminescent medium layer, and a halogenated metal layer or a metal oxide layer is preferably placed on a cathode surface of a light-emitting medium layer. The operation stability for the organic electroluminescent device may be obtained by the surface layer. Preferred examples of the chalcogenide include SiOX (1≤X≤2), AIOX (1≤X≤1.5), SiON, SiAlON, etc; preferred examples of the metal halide include LiF, MgF2, CaF2, rare earth fluoride metal, etc; and preferred examples of the metal oxide include CS₂O, Li₂O, MgO, SrO, BaO, CaO, etc.

The organic electroluminescent device according to one embodiment may further comprise one or more dopants in the light-emitting layers 240, 340. As the dopants included in the organic electroluminescent device of the present disclosure, one or more phosphorescent or fluorescent dopants can be used.

A compound represented by the following formula 100 may be used as a dopant included in the organic electroluminescent device of the present disclosure, but is not limited thereto.

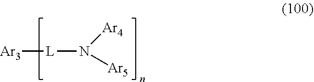

In formula 100,
L is a single bond, substituted or unsubstituted (C6-C30)arylene, or a substituted or unsubstituted (3- to 30-membered)heteroarylene;
Ar₄ and Ar₅ each independently represent a substituted or unsubstituted (C1-C30)alkyl, a substituted or unsubstituted (C6-C30)aryl, a substituted or unsubstituted (3-to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a fused ring of substituted or unsubstituted (C3-C30)aliphatic ring and (C6-C30)aromatic ring, or -L₄-N(Ar₁₃)(Ar₁₄); or Ar₄ and Ar₅ may be linked together to form a ring;
n is an integer from 0 to 2, and when n is 0, Ar₃ is represented by the following formula 100-1, when n is 2,

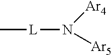

may be the same or different from each other; and

Ar₃ is represented by any one of the following formulas 100-1 to 100-5.

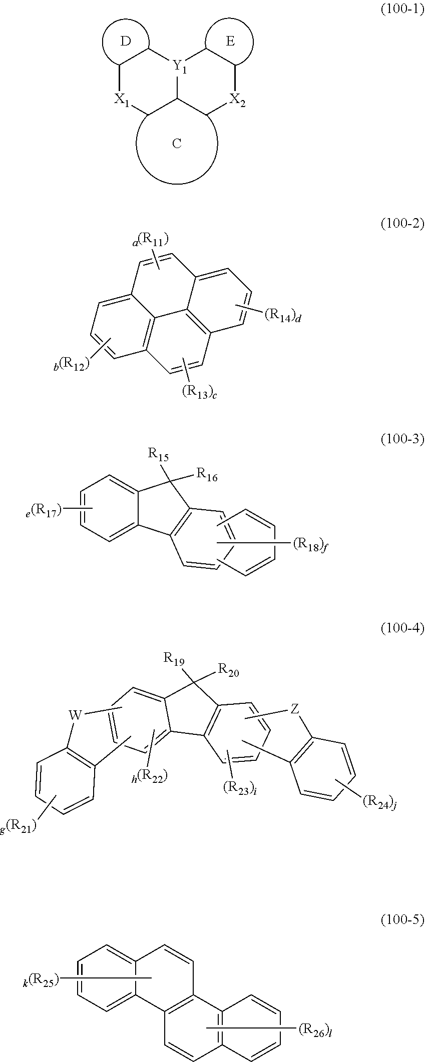

In formulas 100-1 to 100-5,
Y₁ is B;
X₁ and X₂ are each independently NR′, O, or S;
W and Z are each independently O, S, NR′, or CR₂₇R₂₈,
R′ is hydrogen, deuterium, halogen, cyano, a substituted or unsubstituted (C1-C30)alkyl, a substituted or unsubstituted (C6-C30)aryl, a substituted or unsubstituted (3-to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring of (C3-C30)aliphatic ring and (C6-C30)aromatic ring, or -L₄-N(Ar₁₃)(Ar₁₄), and R′ may be directly connected to one or more of C, D, and E rings, or may be connected to form a ring with B, O, S, or CR₂₇R₂₈ as a linker;
Rings C, D, and E are each independently a substituted or unsubstituted (C6-C30)aryl, or a substituted or unsubstituted (3- to 50-membered)heteroaryl; the D and E rings may be directly connected to each other, or connected to form a ring with B, O, S, or CR₂₇R₂₈ as a linker;
R₁₁ to R₁₄, R₁₇, R₁₈, and R₂₁ to R₂₆ are each independently selected from hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring of (C3-C30)aliphatic ring and (C6-C30) aromatic ring, or -LA-N(Ar₁₃)(Ar₁₄);
R₁₅, R₁₆, R₁₉, R₂₀, R₂₇, and R₂₈ are each independently a substituted or unsubstituted (C1-C30)alkyl, a substituted or unsubstituted (C6-C30)aryl, or a substituted or unsubstituted (3- to 30-membered)heteroaryl, wherein at least one of R₁₅ and R₁₆, R₁₉ and R₂₀, and R₂₇ and R₂₈ may be fused together to form a spiro structure;
L₄ is a single bond, a substituted or unsubstituted (C6-C30)arylene, a substituted or unsubstituted (3- to 30-membered)heteroarylene, a substituted or unsubstituted bivalent (C2-C30)aliphatic hydrocarbon group, or a bivalent fused ring of a (C3-C30)aliphatic ring and a (C6-C30) aromatic ring;
Ar₁₃ and Ar₁₄ are each independently a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl;
a, c, h, and i are each independently an integer of 1 or 2; b and d are each independently an integer of 1 to 3; f, k, and l are each independently an integer of 1 to 6; and e, g, and j are each independently an integer of 1 to 4;
when a to l are 2 or more, each of R₁₁ to R₁₄, R₁₇, R₁₈, and R₂₁ to R₂₆ may be the same or different from each other; and
C ring, D ring, E ring, and R₁₁ to R₁₄, R₁₇, R₁₈, and R₂₁ to R₂₆ may have positions that are linked to L of formula 100.

A first electrode 110 or a second electrode 410 formed on a substrate, followed by forming the light-emitting units 200, 300 by any one of dry film methods such as vacuum deposition, sputtering, plasma, ion plating, etc., or wet film-forming methods such as ink jet printing, nozzle printing, slot coating, spin coating, dip coating, flow coating, etc. Thereafter, the charge generation layer 500 and the second electrode 410 or the first electrode 110 are formed thereon to form the organic electroluminescent devices 10, 20 of the present disclosure.

For the wet film method, the materials forming each layer are dissolved or dispersed in a suitable solvent, such as ethanol, chloroform, tetrahydrofuran, or dioxane, to form a thin film, which can be any solvent in which the materials forming each layer can be dissolved or dispersed, and in which there is no problem with filmability.

According to one embodiment, the present disclosure enables the manufacture of display devices such as a smartphone, tablet, laptop, PC, TVs, or display devices for a vehicle, or a lighting device such as, an outdoor or indoor lighting device, using an organic electroluminescent device comprising a compound represented by formula 1.

Hereinafter, for a detailed understanding of the present disclosure, a preparation method of the compound according to the present disclosure will be explained using the synthesis method of a representative compound or intermediate compound of the present disclosure as an example.

### Example 1: Synthesis of Compound C-1

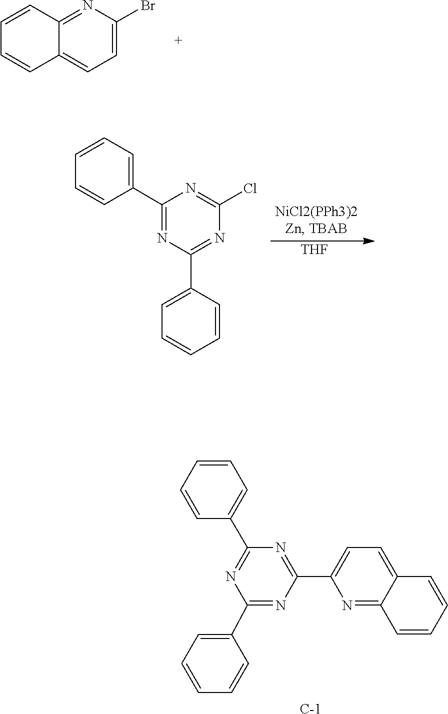

Dichlorobis(triphenylphosphine)nickel (II) (“NiCl₂(PPh₃)₂”) (11.7 g, 21.6 mmol), Zn (10.3 g, 158 mmol), and tetrabutylammonium bromide (“TBAB”) (19.3 g, 60.0 mmol) were added to 400 mL of tetrahydrofuran (“THF”) and stirred. After 30 minutes, 2-bromoquinoline (6.24 g, 30 mmol) and 2,4-diphenyl-6-chloro-1,3,5-triazine (8.0 g, 30 mmol) were added stirred at reflux. After 17 hours, it was cooled to room temperature, filtered through celite, and separated by column chromatography using chloroform/ethyl acetate (“Chloroform/EA”) to obtain compound C-1 (4.2 g, yield: 39%).

| Compound | MW | M.P. |
|---|---|---|
| C-1 | 360.4 | 209.4° C. |

### Example 2: Synthesis of Compound C-2

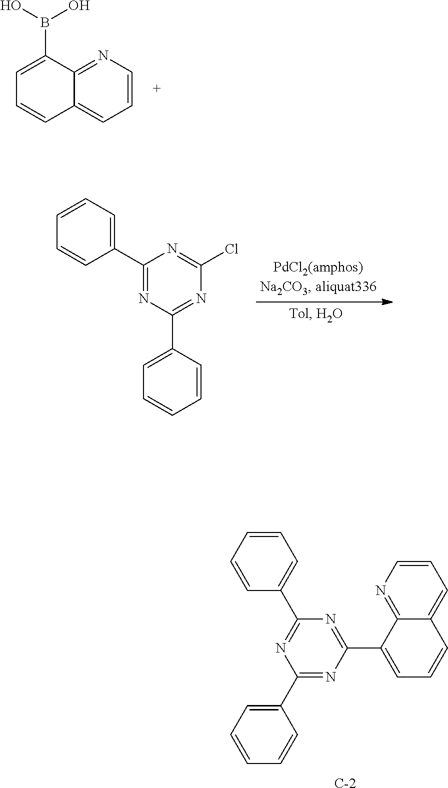

Quinolinyl-8-boronic acid (14 g, 48 mmol), 2-chloro-4,6-diphenyl-1,3,5-triazine (11 g, 40 mmol), PdCl₂ (amphos) (2.8 g, 4.0 mmol), Na₂CO₃ (8.5 g, 80 mmol), and Aliquot336 (1.6 g, 4.0 mmol) were added to 210 ml of toluene, 70 mL of H₂O, and stirred at reflux. After 18 hours, it was cooled to room temperature, filtered through celite, and separated by column chromatography using methylene chloride/tetrahydrofuran (“MC/THF”) to obtain compound C-2 (10 g, yield: 69%).

| Compound | MW | M.P. |
|---|---|---|
| C-2 | 360.4 | 194° C. |

### Example 3: Synthesis of Compound C-75

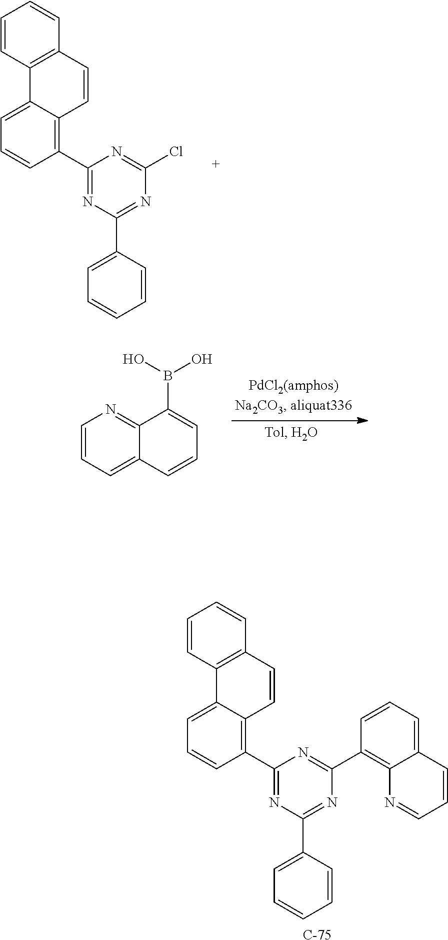

Quinolinyl-8-boronic acid (6.9 g, 24 mmol), 2-chloro-4-(phenanthrene-1-yl)-6-phenyl-1,3,5-triazine (“2-chloro-4-(phenanthrene-1-yl)-6-phenyl-1,3,5-triazine”) (7.3 g, 20 mmol), PdCl₂ (amphos) (1.4 g, 2.0 mmol), Na₂CO₃ (4.2 g, 40 mmol), and Aliquot336 (0.81 g, 2.0 mmol) were added to 300 mL of toluene and 100 mL of H₂O, and stirred at reflux. After 18 hours, it was cooled to room temperature, filtered through celite, and separated by column chromatography using dichloromethane/hexane (“DCM/Hexane”) to obtain compound C-75 (7.0 g, yield: 76%).

| Compound | MW | M.P. |
|---|---|---|
| C-75 | 460.5 | 173.8° C. |

### Example 4: Synthesis of Compound C-97

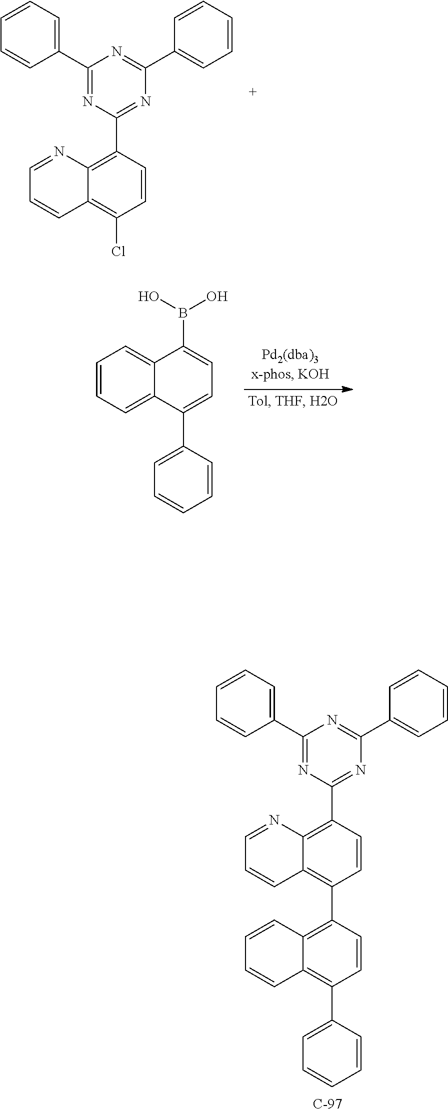

5-chloro-8-(4,6-diphenyl-1,3,5-triazin-2-yl)quinoline (7.9 g, 20 mmol), (4-phenylnaphthalene-1-yl)boronic (5.0 g, 20 mmol), Pd₂(dba)₃ (0.2 g, 0.2 mmol), x-phos (0.2 g, 0.4 mmol), and KOH (1.8 g, 40 mmol) were added to 250 mL of toluene, 100 ml of tetrahydrofuran, and 50 ml of H₂O, and stirred at 70° C. After 18 hours, it was cooled to room temperature, filtered through celite, and separated by column chromatography using toluene/EtOH to obtain compound C-97 (6.2 g, yield:55%).

| Compound | MW | M.P. |
|---|---|---|
| C-97 | 562.2 | 230° C. |

### Example 5: Synthesis of Compound C-77

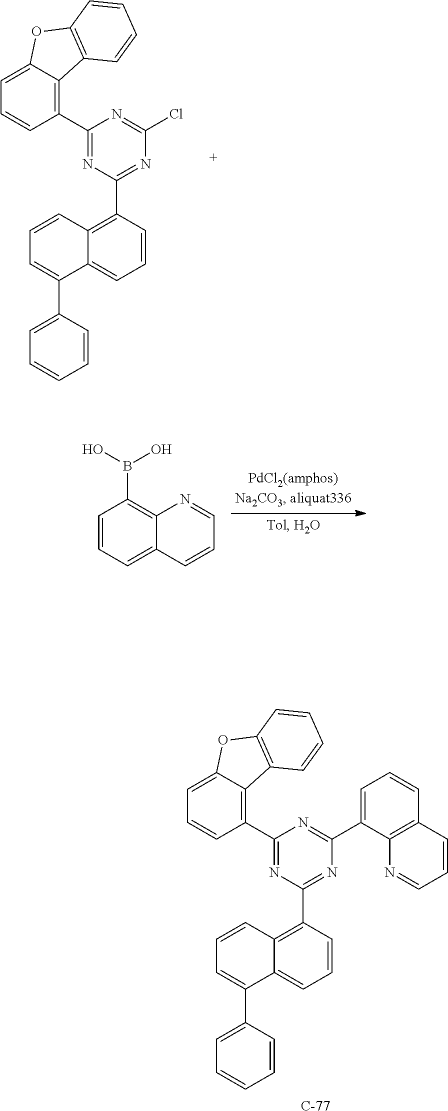

Quinolinyl-8-boronic acid (5.2 g, 18 mmol), 2-chloro-4-(dibenzo[b,d]furan-1-yl)-6-(5-phenylnaphthalene-1-yl)-1,3,5-triazine (7.3 g, 15 mmol), PdCl₂ (amphios) (1.1 g, 1.5 mmol), Na₂CO₃ (3.2 g, 30 mmol), and Aliquot336 (0.61 g, 1.5 mmol) were added to toluene 105 mL, H₂O 35 mL, and stirred at reflux. After 18 hours, it was cooled to room temperature, filtered through celite, and separated by column chromatography using dichloromethane to obtain compound C-77 (7.5 g, yield: 87%).

| Compound | MW | M.P. |
|---|---|---|
| C-77 | 576.2 | 179.7° C. |

### Example 6: Synthesis of Compound C-99

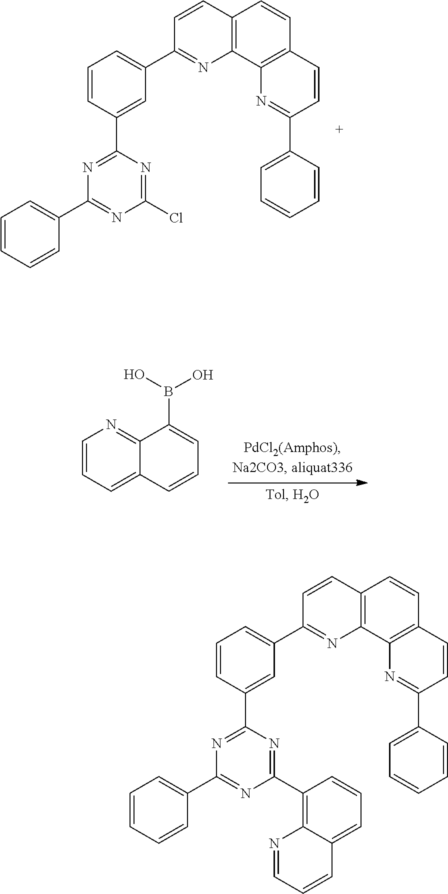 [SMILES: Clc1nc(-c2ccccc2)nc(-c2cccc(-c3ccc4ccc5ccc(-c6ccccc6)nc5c4n3)c2)n1 ; OB(O)c1cccc2cccnc12 ; c1ccc(-c2ccc3ccc4ccc(-c5cccc(-c6nc(-c7ccccc7)nc(-c7cccc8cccnc78)n6)c5)nc4c3n2)cc1]

2-(3-(4-chloro-6-phenyl-1,3,5-triazin-2-yl)phenyl)-9-phenyl-1,10-phenanthroline (16.3 g, 31.22 mmol), quinolinyl-8-boronic acid (6.5 g, 37.47 mmol), PdCl₂ (amphos) (2.2 g, 3.12 mmol), Aliquot336 (1.3 g, 3.12 mmol), and Na₂CO₃ (6.6 g, 62.44 mmol) were dissolved in 210 mL of toluene, 70 mL of H₂O, and stirred at reflux at 130° C. After 2 hours, the solid-produced reaction product was cooled to room temperature, filtered through silica, and recrystallized to obtain compound C-99 (6.1 g, yield: 31.93%).

| Compound | MW | M.P. |
|---|---|---|
| C-99 | 460.53 | 237° C. |

For a detailed understanding of the present disclosure, a preparation method of an organic electroluminescent device comprising the aforementioned organic electroluminescent material and characteristics thereof will be described.

### Device Examples 1 to 6: Preparation of OLEDs Deposited with a Compound According to the Present Disclosure as a Charge Generation Layer

OLEDs according to the present disclosure were prepared. First, a transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and then was stored in isopropyl alcohol. The ITO substrate was mounted on a substrate holder of a vacuum vapor deposition apparatus. Compound HI-1 was introduced into a cell of the vacuum vapor deposition apparatus, and compound HT-1 was introduced into another cell. The two materials were evaporated at different rates, and compound HI-1 was deposited in a doping amount of 7 wt % based to the total amount of compound HI-1 and compound HT-1 to form a hole injection layer with a thickness of 5 nm. Subsequently, compound HT-1 was deposited on the hole injection layer to form a first hole transport layer with a thickness of 30 nm. Next, compound HT-2 was introduced into another cell of the vacuum vapor deposition apparatus and was evaporated by applying an electric current to the cell, thereby depositing a second hole transport layer with a thickness of 5 nm on the first hole transport layer. After forming the hole injection layer and the hole transport layers, a first light-emitting layer was deposited thereon as follows: After placing compound H-1 as a host in a cell in the vacuum deposition equipment and compound D-1 as a dopant in another cell, the two substances were evaporated at different rates to dope the dopant in an amount of 2 wt % with respect to the total amount of host and dopant, to form a first light-emitting layer having a thickness of 20 nm on the second hole transport layer. Next, 5 nm of compound ET-1 was deposited as the first hole blocking layer material on the first light-emitting layer. Then, a first electron transport layer with a thickness of 25 nm was deposited by doping compound ET-2:EI-1 in a weight ratio of 1 as the electron transport layer material. Thereafter, a n-type charge generation layer of 10 nm was formed by depositing 1 wt % of Li on the compounds in Table 1 below. Then, a p-type charge generation layer of 10 nm thickness was deposited by doping compound HI-1 in an amount of 15 wt % with respect to the total amount of compound HI-1 and compound HT-1. Next, 30 nm of compound HT-1 was deposited to form a third hole transport layer, followed by 5 nm of compound HT-2 to form a fourth hole transport layer. A second light-emitting layer was then deposited as follows: after adding compound H-1 as a host and compound D-1 as a dopant to the cell in the vacuum deposition equipment, the two substances were evaporated at different rates to dope the dopant in an amount of 2 wt % relative to the total amount of host and dopant to form a second light-emitting layer 20 nm thickness on the fourth hole transport layer. On the second light-emitting layer, 5 nm of compound ET-1 was deposited as the second hole blocking layer material, and then compounds ET-2 and EI-1 were respectively added to two cells in the vacuum deposition equipment as a second electron transport layer material, and then the two materials were deposited to a thickness of 25 nm at a weight ratio of 2:1. After depositing Yb as an electron injection layer with a thickness of 1 nm on the second electron transport layer, an Al cathode was deposited to a thickness of 80 nm on the electron injection layer by using another vacuum vapor deposition apparatus. Thus, OLEDs were produced. Each compounds used for all the materials were purified by vacuum sublimation under 10-6 torr.

### Device Comparative Example 1: Preparation of OLED with a Conventional Compound Deposited as a Charge Generation Layer

Except for using the compound in Table 1 below as the n-type charge generation layer material, OLED was prepared in the same way as in device example 1.

The driving voltage, luminous efficiency, and the amount of progressive driving voltage change (ΔV) at a luminance of 1,000 nits of the organic electroluminescent device of Device Examples 1 to 6 and Device Comparative Example 1 prepared as described above, were measured, respectively, and the results thereof are shown in the following Table 1.

TABLE 1

| | n-type Charge | | Luminous | |
|---|---|---|---|---|
| | Generation | Driving | Efficiency | ΔV |
| | Layer | Voltage [V] | [cd/A] | (%) |
| Device | C-2 | 6.45 | 8.5 | 103.5 |
| Example 1 | | | | |
| Device | C-1 | 6.38 | 8.6 | 103.3 |
| Example 2 | | | | |
| Device | C-97 | 6.20 | 8.7 | 101.2 |
| Example 3 | | | | |
| Device | C-75 | 6.20 | 8.6 | 101.3 |
| Example 4 | | | | |
| Device | C-77 | 6.29 | 8.7 | 101.1 |
| Example 5 | | | | |
| Device | C-74 | 6.34 | 8.6 | 101.4 |
| Example 6 | | | | |
| Device | ref-1 | 6.92 | 8.2 | 105.4 |
| Comparative | | | | |
| Example 1 | | | | |

From Table 1, it can be seen that the organic electroluminescent device including the compound according to the present disclosure in the n-type charge generation layer has significantly improved luminous efficiency and/or lifespan compared to the conventional organic electroluminescent device.

The compounds used in the Device Examples 1 to 6 and the Device Comparative Example 1 are shown in Table 2 below.

TABLE 2

| | |
|---|---|
| Hole injection layer/ Hole transport layer | 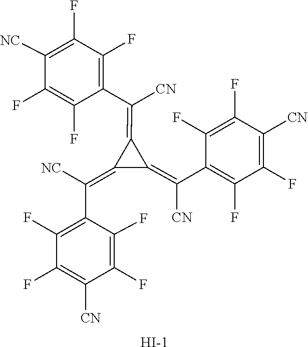 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; [I-]] |
| | 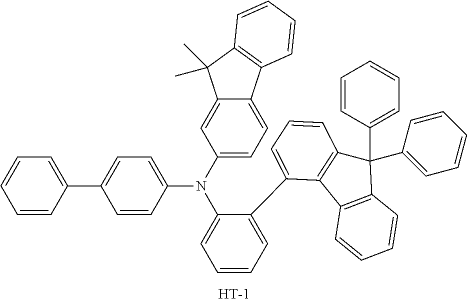 |
| | 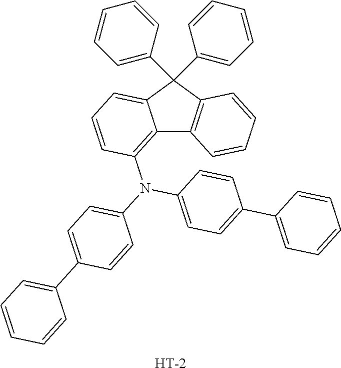 |
| Light-emitting layer | 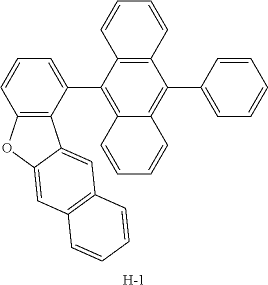 |
| | 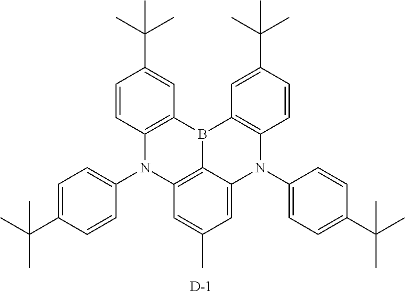 |
| Electron transport layer/ Hole blocking layer/ Electron injection layer | 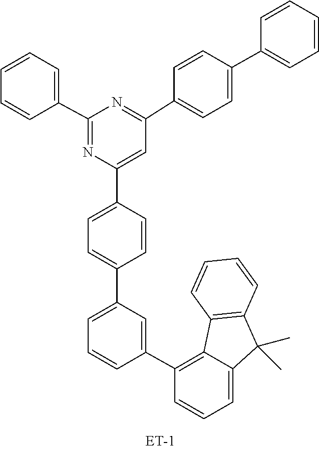 [SMILES: CC1(C)c2ccccc2-c2c(-c3cccc(-c4ccc(-c5cc(-c6ccc(-c7ccccc7)cc6)nc(-c6ccccc6)n5)cc4)c3)cccc21] |
| | 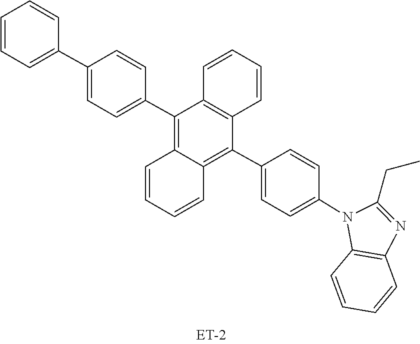 [SMILES: CCc1nc2ccccc2n1-c1ccc(-c2c3ccccc3c(-c3ccc(-c4ccccc4)cc3)c3ccccc23)cc1] |
| | 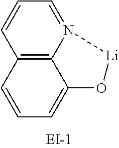 |
| n-type charge generation layer | 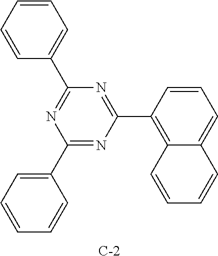 |
| | 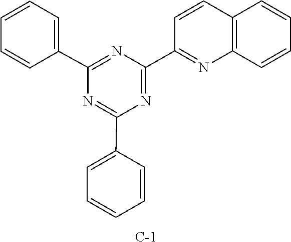 |
| | 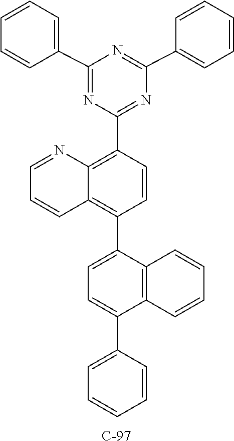 |
| | 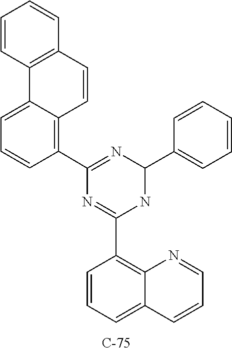 |
| | 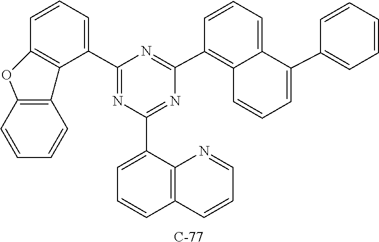 |
| | 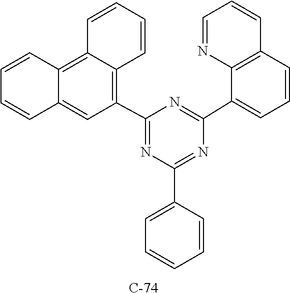 |
| | 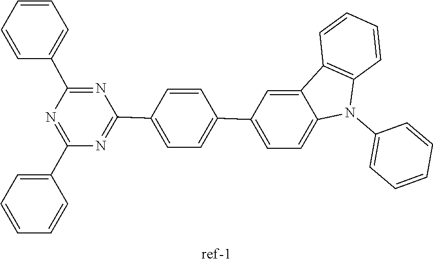 [SMILES: c1ccc(-c2nc(-c3ccccc3)nc(-c3ccc(-c4ccc5c(c4)c4ccccc4n5-c4ccccc4)cc3)n2)cc1] |

### Device Examples 7 and 8: Preparation of OLEDs Deposited with a Compound According to the Present Disclosure as a Charge Generation Layer

OLEDs according to the present disclosure were prepared. First, a transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and then was stored in isopropyl alcohol. The ITO substrate was mounted on a substrate holder of a vacuum vapor deposition apparatus. Compound HI-1 was introduced into a cell of the vacuum vapor deposition apparatus, and compound HT-3 was introduced into another cell. The two materials were evaporated at different rates, and compound HI-1 was deposited in a doping amount of 3 wt % based to the total amount of compound HI-1 and compound HT-3 to form a hole injection layer with a thickness of 5 nm. Subsequently, compound HT-1 was deposited on the hole injection layer to form a first hole transport layer with a thickness of 30 nm. Next, compound HT-4 was introduced into another cell of the vacuum vapor deposition apparatus and was evaporated by applying an electric current to the cell, thereby depositing a second hole transport layer with a thickness of 5 nm on the first hole transport layer. After forming the hole injection layer and the hole transport layers, a first light-emitting layer was deposited thereon as follows:

After placing compound H-1 as a host in a cell in the vacuum deposition equipment and compound D-1 as a dopant in another cell, the two substances were evaporated at different rates to dope the dopant in an amount of 2 wt % with respect to the total amount of host and dopant, to form a first light-emitting layer having a thickness of 20 nm on the second hole transport layer. Then, 5 nm of compound ET-1 was deposited as the first hole blocking layer material on the first light-emitting layer.

Then, a first electron transport layer with a thickness of 10 nm was formed by depositing compound ET-3 in a weight ratio of 1 as the electron transport layer material. Thereafter, a n-type charge generation layer of 4 nm was formed by depositing 0.5 wt % of Li on the compounds in Table 4 below. Then, a p-type charge generation layer of 10 nm thickness was deposited by doping compound HI-1 in an amount of 6 wt % with respect to the total amount of compound HI-1 and compound HT-1.

Next, 30 nm of compound HT-1 was deposited to form a third hole transport layer, followed by 5 nm of compound HT-2 to form a fourth hole transport layer. A second light-emitting layer was then deposited as follows: after adding compound H-1 as a host and compound D-1 as a dopant to the cell in the vacuum deposition equipment, the two substances were evaporated at different rates to dope the dopant in an amount of 2 wt % relative to the total amount of host and dopant to form a second light-emitting layer 20 nm thickness on the fourth hole transport layer. On the second light-emitting layer, 5 nm of compound ET-1 was deposited as the second hole blocking layer material, and then compounds ET-2 and EI-1 were respectively added to two cells in the vacuum deposition equipment as a second electron transport layer material, and then the two materials were deposited to a thickness of 25 nm at a weight ratio of 2:1. After depositing Yb as an electron injection layer with a thickness of 1 nm on the second electron transport layer, an Al cathode was deposited to a thickness of 80 nm on the electron injection layer by using another vacuum vapor deposition apparatus. Thus, OLEDs were produced. Each compounds used for all the materials were purified by vacuum sublimation under 10-6 torr.

### Device Comparative Example 2: Preparation of OLEDs with a Conventional Compound Deposited as a Charge Generation Layer

Except for using the compound in Table 3 below as the n-type charge generation layer material, OLEDs were prepared in the same way as in device example 7.

The driving voltage, the time taken for the luminance to decrease from 100% to 95% (lifespan; T95), and the amount of progressive driving voltage change (ΔV) at a luminance of 1,000 nits of the organic electroluminescent device of Device Examples 7 and 8 and Device Comparative Example 2 prepared as described above, were measured, respectively, and the results thereof are shown in the following Table 3.

TABLE 3

| | n-type Charge | Driving | Lifespan T95 | ΔV |
|---|---|---|---|---|
| | Generation Layer | Voltage [V] | [hr] | (%) |
| Device | C-77 | 7.7 | 158 | 102.9 |
| Example 7 | | | | |
| Device | C-99 | 6.5 | 144 | 101.4 |
| Example 8 | | | | |
| Device | ref-1 | 7.5 | 3 | 126.9 |
| Comparative | | | | |
| Example 2 | | | | |

From Table 3, it can be seen that the organic electroluminescent device including the compound according to the present disclosure in the n-type charge generation layer has significantly improved lifespan compared to the conventional organic electroluminescent device.

The compounds used in the Device Examples 7 and 8 and the Device Comparative Example 2 are shown in Table 4 below.

TABLE 4

| | |
|---|---|
| Hole injection layer/ Hole transport layer | 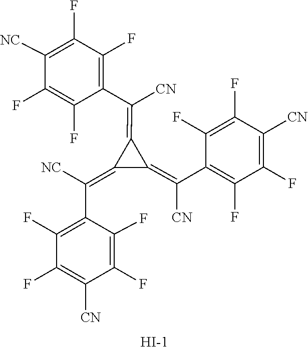 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; [I-]] |
| | 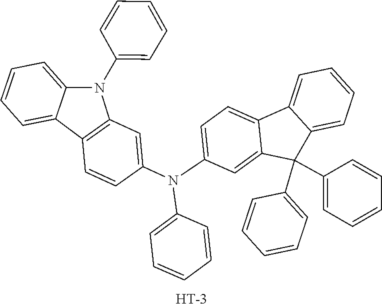 |
| | 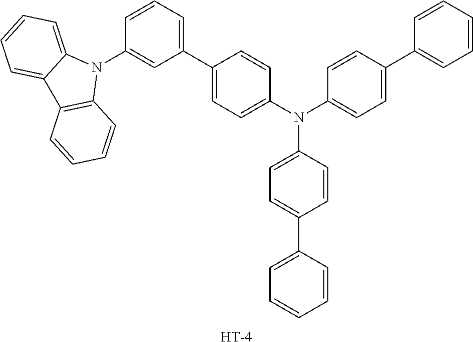 |
| Light-emitting layer | 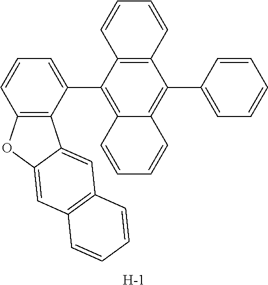 |
| | 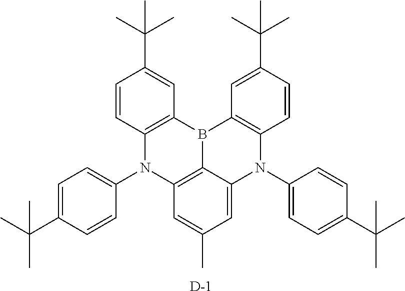 |
| Electron transport layer/ Hole blocking layer/ Electron injection layer | 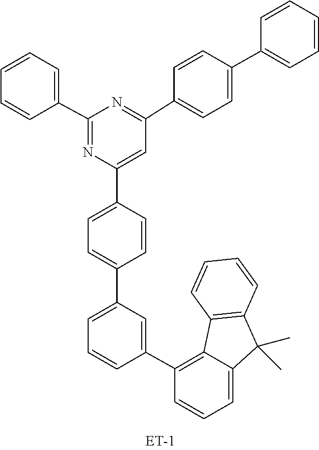 [SMILES: CC1(C)c2ccccc2-c2c(-c3cccc(-c4ccc(-c5cc(-c6ccc(-c7ccccc7)cc6)nc(-c6ccccc6)n5)cc4)c3)cccc21] |
| | 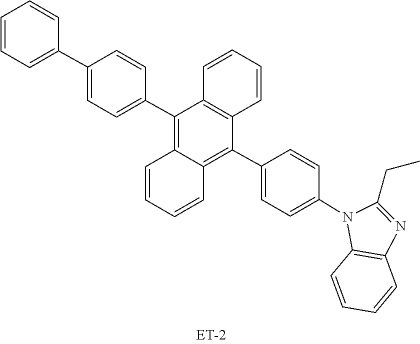 [SMILES: CCc1nc2ccccc2n1-c1ccc(-c2c3ccccc3c(-c3ccc(-c4ccccc4)cc3)c3ccccc23)cc1] |
| | 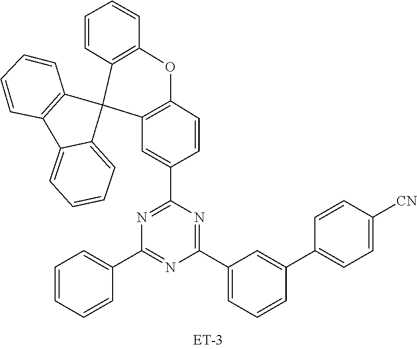 [SMILES: N#Cc1ccc(-c2cccc(-c3nc(-c4ccccc4)nc(-c4ccc5c(c4)C4(c6ccccc6O5)c5ccccc5-c5ccccc54)n3)c2)cc1] |
| | 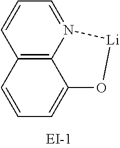 |
| n-type charge generation layer | 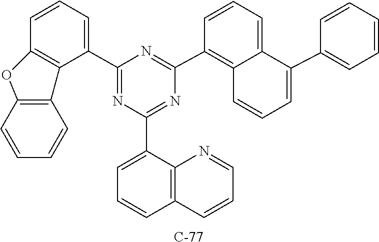 |
| | 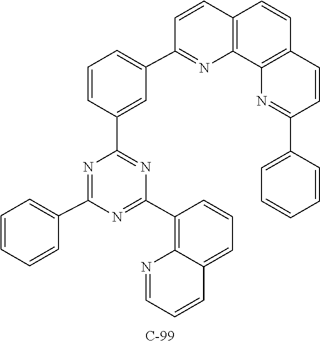 |
| | 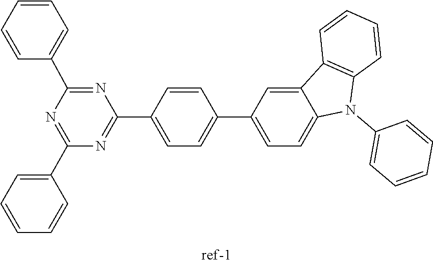 [SMILES: c1ccc(-c2nc(-c3ccccc3)nc(-c3ccc(-c4ccc5c(c4)c4ccccc4n5-c4ccccc4)cc3)n2)cc1] |

### EXPLANATION OF SYMBOLS

10, 20: Organic electroluminescent device
110: First electrode
200, 300: Light emitting unit
210: Hole injection layer
220, 230, 320, 330: Hole transport layer
240, 340: Light-emitting layer
250, 350: Hole blocking layer
260, 360: Electron transport layer
370: Electron injection layer
410: Second electrode
500: Charge generation layer
510: n-type charge generation layer
520: p-type charge generation layer

1. An organic electroluminescent device comprising: a plurality of light emitting units disposed between a first electrode and a second electrode and comprising at least one light-emitting layer(s); and at least one n-type charge generation layer(s) disposed between the adjacent light-emitting units, wherein the n-type charge generation layer comprises a triazinyl quinoline derivative compound.

2. The organic electroluminescence device according to claim 1, wherein the triazinyl quinoline derivative compound is represented by the following formula 1: 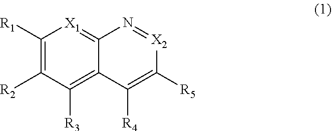
In formula 1,
R₁ to R₅ each independently represent, hydrogen, deuterium, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, or a substituted or unsubstituted tri(C6-C30)arylsilyl;
X₁ and X₂ each independently represent CR₈ or are represented by the following formula 2, and one of X₁ and X₂ is represented by the following formula 2, with the proviso that the case where both X₁ and X₂ are represented by the following formula 2 is excluded;
R₈ represents hydrogen, deuterium, or a substituted or unsubstituted (C6-C30)aryl, and 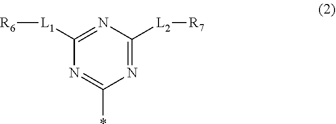
In formula 2,
L₁ and L₂ each independently represent a single bond, a substituted or unsubstituted (C6-C30)arylene, a substituted or unsubstituted (3- to 30-membered)heteroarylene, or a substituted or unsubstituted (C3-C30)cycloalkylene;
R₆ and R₇ each independently represent a substituted or unsubstituted (C1-C30)alkyl, a substituted or unsubstituted (C6-C30)aryl, a substituted or unsubstituted (3-to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, or a substituted or unsubstituted tri(C6-C30)arylsilyl; and
* represents a carbon atom attached to formula 1.

3. The organic electroluminescence device according to claim 2, wherein R₁ to R₅ are each independently selected from hydrogen, deuterium, a substituted or unsubstituted phenyl, a substituted or unsubstituted naphthyl, or a combination thereof, and the substituted phenyl or the substituted naphthyl is substituted with at least one selected from deuterium and a phenyl substituted with deuterium, and
wherein R₈ is hydrogen, deuterium, or a substituted or unsubstituted phenyl, and the substituted phenyl is substituted with deuterium.

4. The organic electroluminescence device according to claim 2, wherein L₁ and L₂ are each independently selected from a single bond, a substituted or unsubstituted phenylene, a substituted or unsubstituted naphthylene, a substituted or unsubstituted biphenylene, or a combination thereof, and the substituted phenylene, the substituted naphthylene, or the substituted biphenylene is substituted with deuterium.

5. The organic electroluminescence device according to claim 2, wherein R₆ and R₇ are each independently selected from a substituted or unsubstituted phenyl, a substituted or unsubstituted naphthyl, a substituted or unsubstituted dibenzofuranyl, a substituted or unsubstituted dibenzothiophenyl, a substituted or unsubstituted biphenyl, a substituted or unsubstituted m-terphenyl, a substituted or unsubstituted o-terphenyl, a substituted or unsubstituted dimethylfluorenyl, a substituted or unsubstituted diphenylfluorenyl, a substituted or unsubstituted phenanthrenyl, a substituted or unsubstituted phenanthrolinyl, a substituted or unsubstituted anthracenyl, a substituted or unsubstituted chrysenyl, a substituted or unsubstituted isocrysenyl, a substituted or unsubstituted benzoxazolyl, a substituted or unsubstituted carbazolyl, or a combination thereof, and the substituted phenyl, the substituted naphthyl, the substituted dibenzofuranyl, the substituted dibenzothiophenyl, the substituted biphenyl, the substituted m-terphenyl, the substituted o-terphenyl, the substituted dimethylfluorenyl, the substituted diphenylfluorenyl, the substituted phenanthrenyl, or the substituted chrysenyl is substituted with deuterium, the substituted phenanthrolinyl is substituted with at least one selected from phenyl and naphthyl, and the substituted anthracenyl, the substituted benzoxazolyl, or the substituted carbazolyl is substituted with phenyl.

6. The organic electroluminescence device according to claim 2, wherein the compound represented by formula 1 is selected from the following compounds: 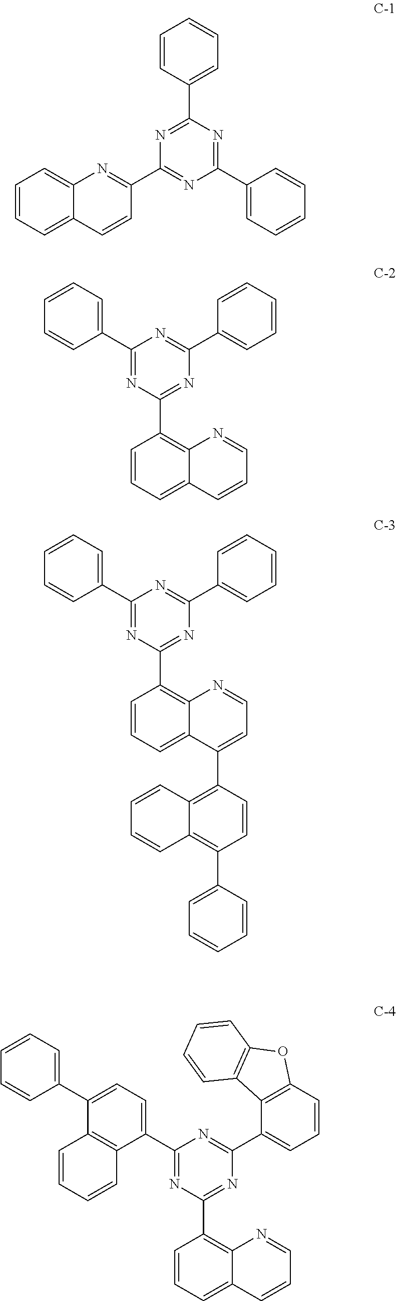 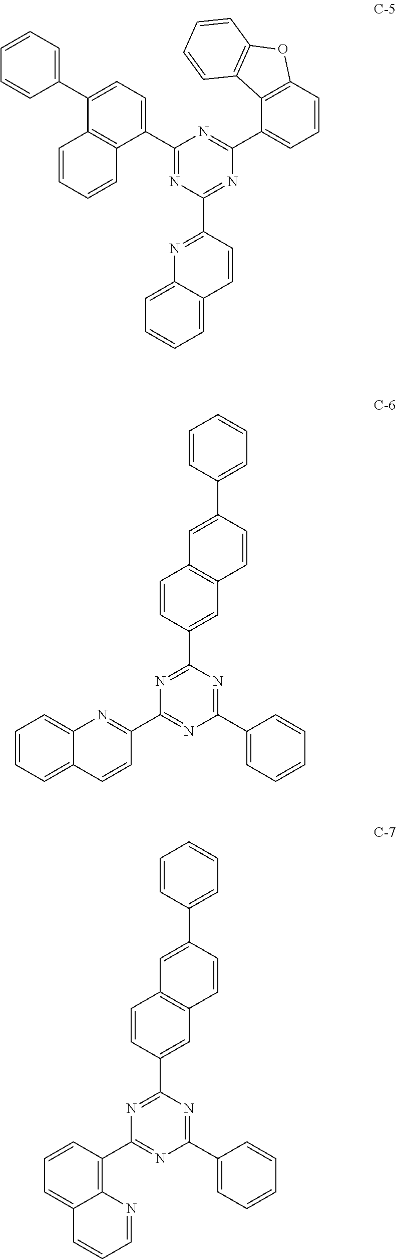 [SMILES: c1ccc(-c2ccc(-c3nc(-c4ccc5ccccc5n4)nc(-c4cccc5oc6ccccc6c45)n3)c3ccccc23)cc1 ; c1ccc(-c2ccc3cc(-c4nc(-c5ccccc5)nc(-c5ccc6ccccc6n5)n4)ccc3c2)cc1 ; c1ccc(-c2ccc3cc(-c4nc(-c5ccccc5)nc(-c5cccc6cccnc56)n4)ccc3c2)cc1] 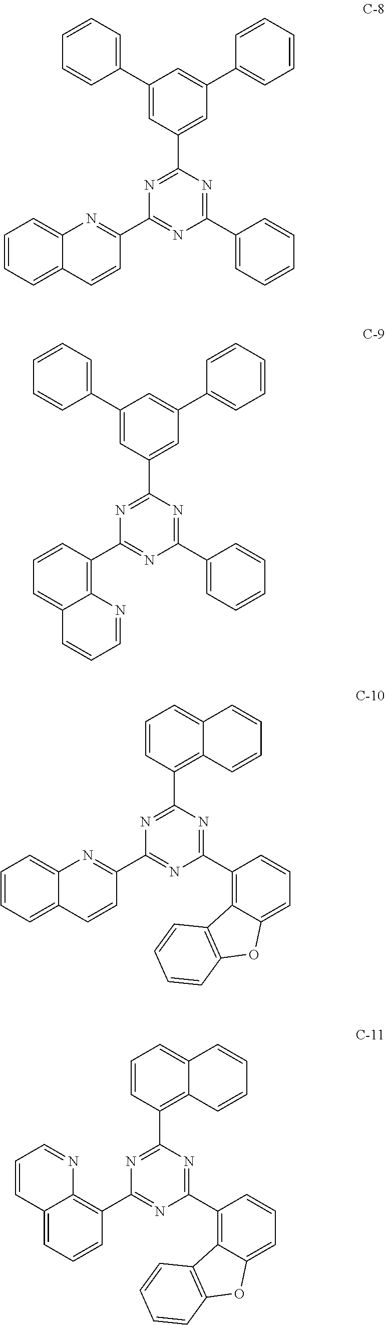 [SMILES: c1ccc(-c2cc(-c3ccccc3)cc(-c3nc(-c4ccccc4)nc(-c4ccc5ccccc5n4)n3)c2)cc1 ; c1ccc(-c2cc(-c3ccccc3)cc(-c3nc(-c4ccccc4)nc(-c4cccc5cccnc45)n3)c2)cc1 ; c1ccc2nc(-c3nc(-c4cccc5ccccc45)nc(-c4cccc5oc6ccccc6c45)n3)ccc2c1 ; c1ccc2c(-c3nc(-c4cccc5cccnc45)nc(-c4cccc5oc6ccccc6c45)n3)cccc2c1] 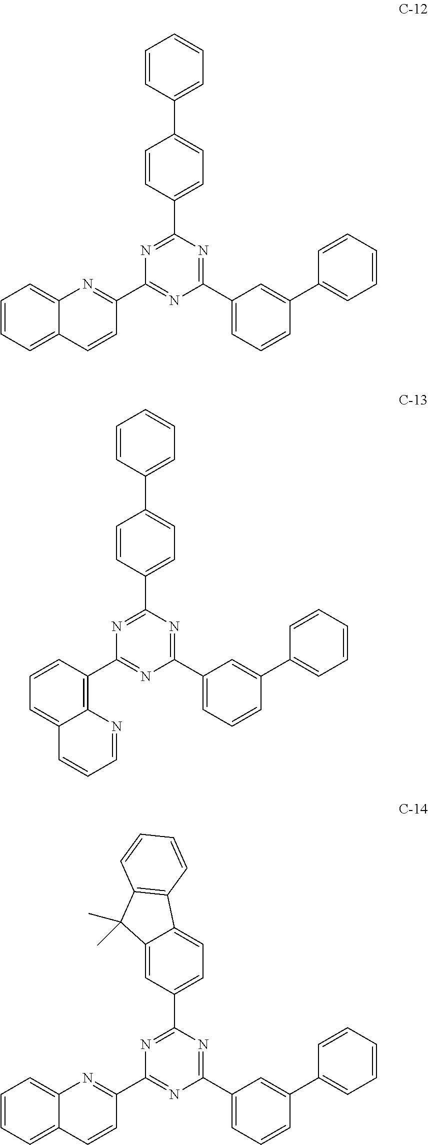 [SMILES: c1ccc(-c2ccc(-c3nc(-c4cccc(-c5ccccc5)c4)nc(-c4ccc5ccccc5n4)n3)cc2)cc1 ; c1ccc(-c2ccc(-c3nc(-c4cccc(-c5ccccc5)c4)nc(-c4cccc5cccnc45)n3)cc2)cc1 ; CC1(C)c2ccccc2-c2ccc(-c3nc(-c4cccc(-c5ccccc5)c4)nc(-c4ccc5ccccc5n4)n3)cc21] 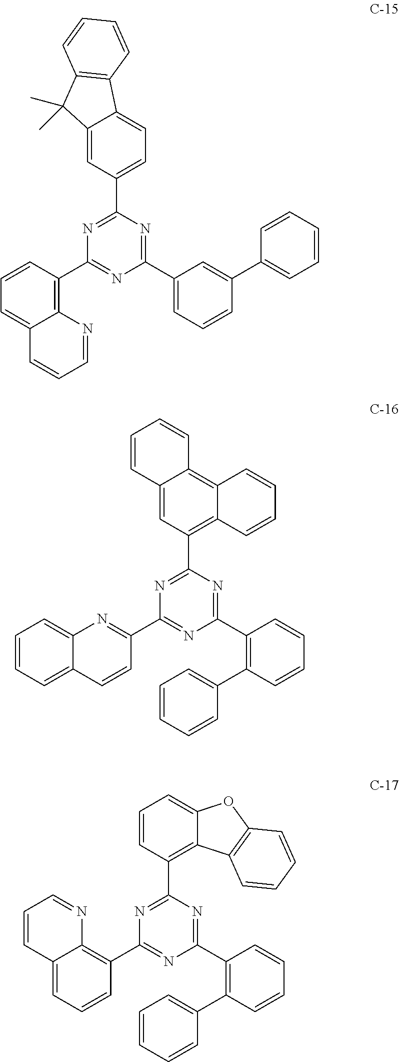 [SMILES: CC1(C)c2ccccc2-c2ccc(-c3nc(-c4cccc(-c5ccccc5)c4)nc(-c4cccc5cccnc45)n3)cc21 ; c1ccc(-c2ccccc2-c2nc(-c3ccc4ccccc4n3)nc(-c3cc4ccccc4c4ccccc34)n2)cc1 ; c1ccc(-c2ccccc2-c2nc(-c3cccc4cccnc34)nc(-c3cccc4oc5ccccc5c34)n2)cc1] 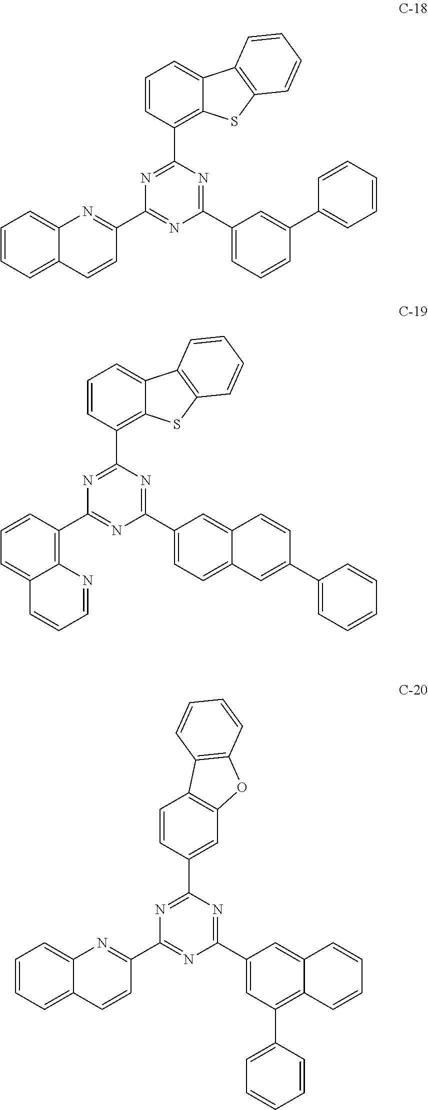 [SMILES: c1ccc(-c2cccc(-c3nc(-c4ccc5ccccc5n4)nc(-c4cccc5c4sc4ccccc45)n3)c2)cc1 ; c1ccc(-c2ccc3cc(-c4nc(-c5cccc6cccnc56)nc(-c5cccc6c5sc5ccccc56)n4)ccc3c2)cc1 ; c1ccc(-c2cc(-c3nc(-c4ccc5c(c4)oc4ccccc45)nc(-c4ccc5ccccc5n4)n3)cc3ccccc23)cc1] 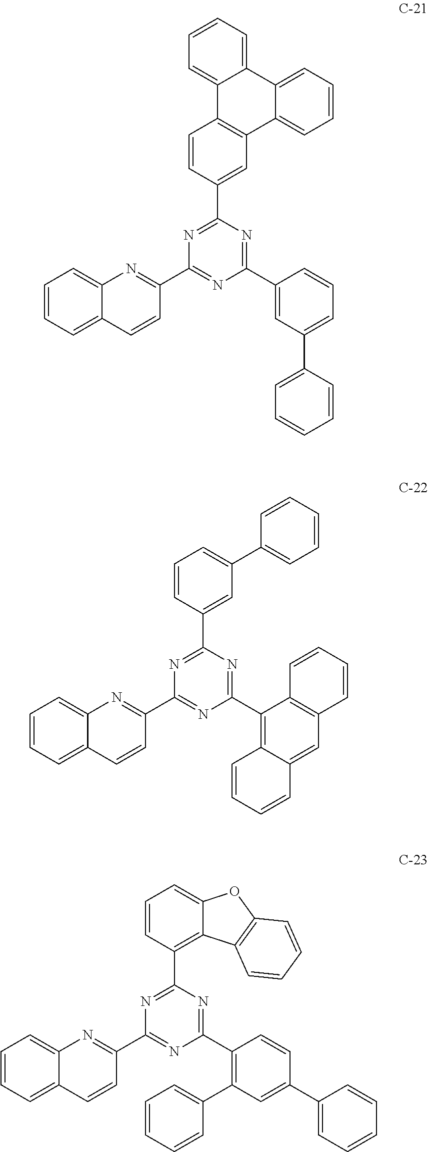 [SMILES: c1ccc(-c2cccc(-c3nc(-c4ccc5c6ccccc6c6ccccc6c5c4)nc(-c4ccc5ccccc5n4)n3)c2)cc1 ; c1ccc(-c2cccc(-c3nc(-c4ccc5ccccc5n4)nc(-c4c5ccccc5cc5ccccc45)n3)c2)cc1 ; c1ccc(-c2ccc(-c3nc(-c4ccc5ccccc5n4)nc(-c4cccc5oc6ccccc6c45)n3)c(-c3ccccc3)c2)cc1] 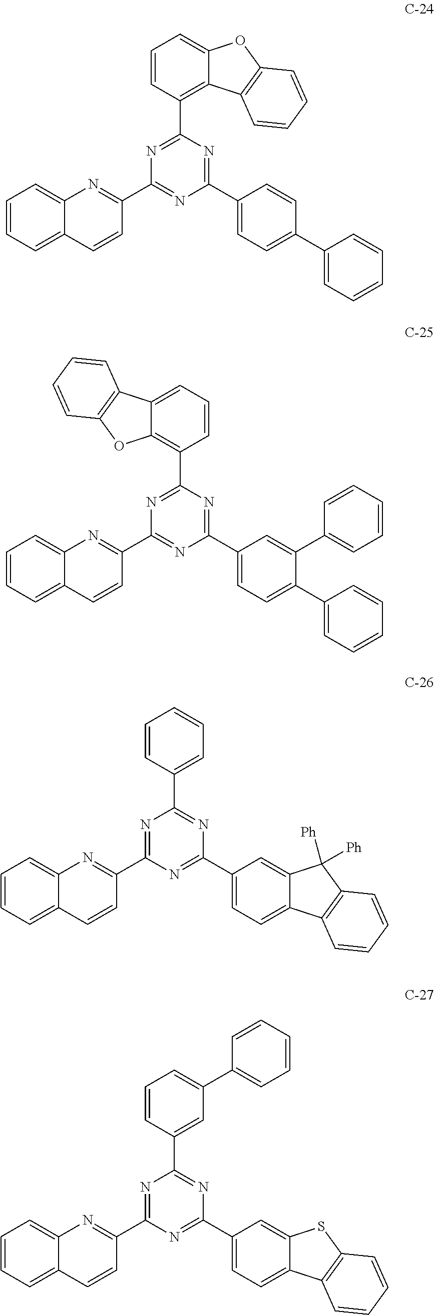 [SMILES: c1ccc(-c2ccc(-c3nc(-c4ccc5ccccc5n4)nc(-c4cccc5oc6ccccc6c45)n3)cc2)cc1 ; c1ccc(-c2ccc(-c3nc(-c4ccc5ccccc5n4)nc(-c4cccc5c4oc4ccccc45)n3)cc2-c2ccccc2)cc1 ; c1ccc(-c2nc(-c3ccc4c(c3)C(c3ccccc3)(c3ccccc3)c3ccccc3-4)nc(-c3ccc4ccccc4n3)n2)cc1 ; c1ccc(-c2cccc(-c3nc(-c4ccc5c(c4)sc4ccccc45)nc(-c4ccc5ccccc5n4)n3)c2)cc1] 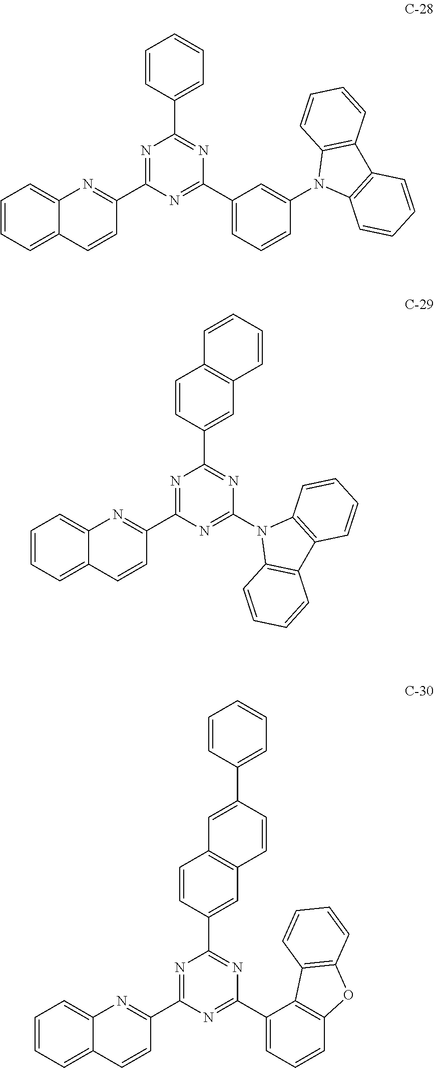 [SMILES: c1ccc(-c2nc(-c3cccc(-n4c5ccccc5c5ccccc54)c3)nc(-c3ccc4ccccc4n3)n2)cc1 ; c1ccc2cc(-c3nc(-c4ccc5ccccc5n4)nc(-n4c5ccccc5c5ccccc54)n3)ccc2c1 ; c1ccc(-c2ccc3cc(-c4nc(-c5ccc6ccccc6n5)nc(-c5cccc6oc7ccccc7c56)n4)ccc3c2)cc1] 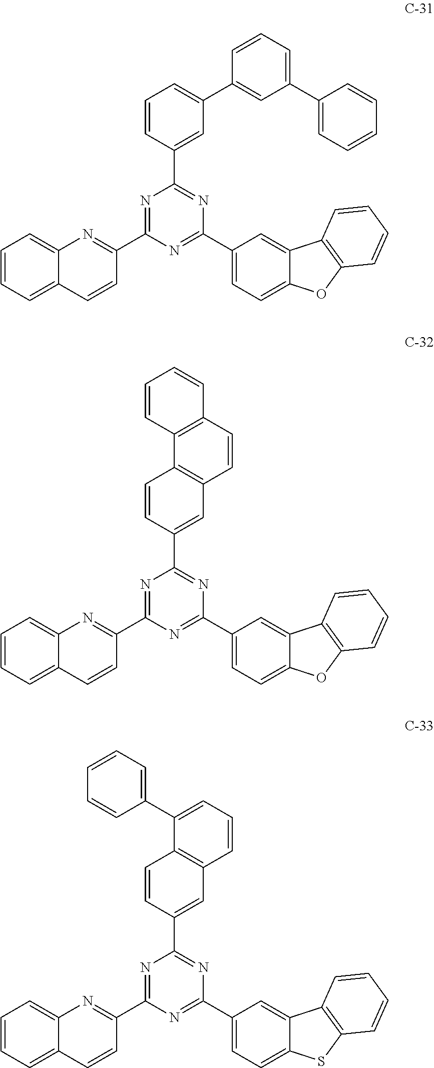 [SMILES: c1ccc(-c2cccc(-c3cccc(-c4nc(-c5ccc6oc7ccccc7c6c5)nc(-c5ccc6ccccc6n5)n4)c3)c2)cc1 ; c1ccc2nc(-c3nc(-c4ccc5c(ccc6ccccc65)c4)nc(-c4ccc5oc6ccccc6c5c4)n3)ccc2c1 ; c1ccc(-c2cccc3cc(-c4nc(-c5ccc6sc7ccccc7c6c5)nc(-c5ccc6ccccc6n5)n4)ccc23)cc1] 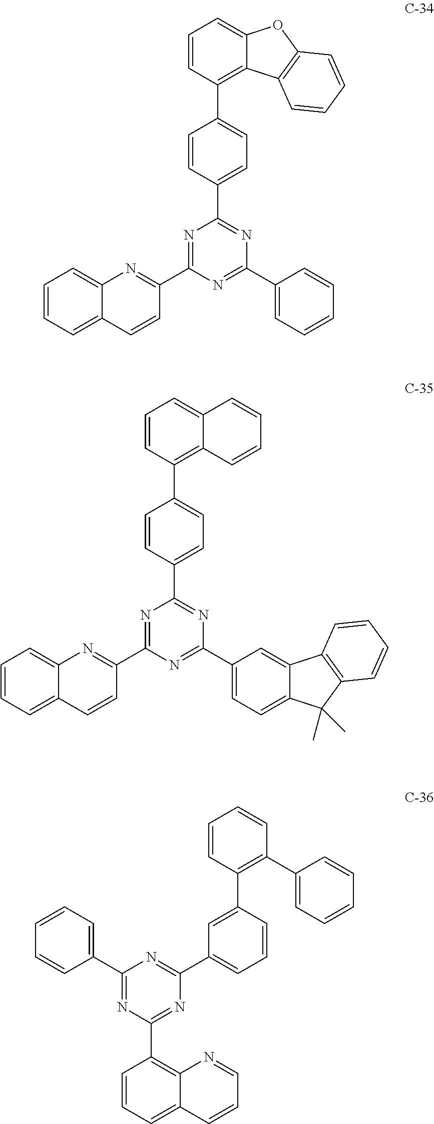 [SMILES: c1ccc(-c2nc(-c3ccc(-c4cccc5oc6ccccc6c45)cc3)nc(-c3ccc4ccccc4n3)n2)cc1 ; CC1(C)c2ccccc2-c2cc(-c3nc(-c4ccc(-c5cccc6ccccc56)cc4)nc(-c4ccc5ccccc5n4)n3)ccc21 ; c1ccc(-c2nc(-c3cccc(-c4ccccc4-c4ccccc4)c3)nc(-c3cccc4cccnc34)n2)cc1] 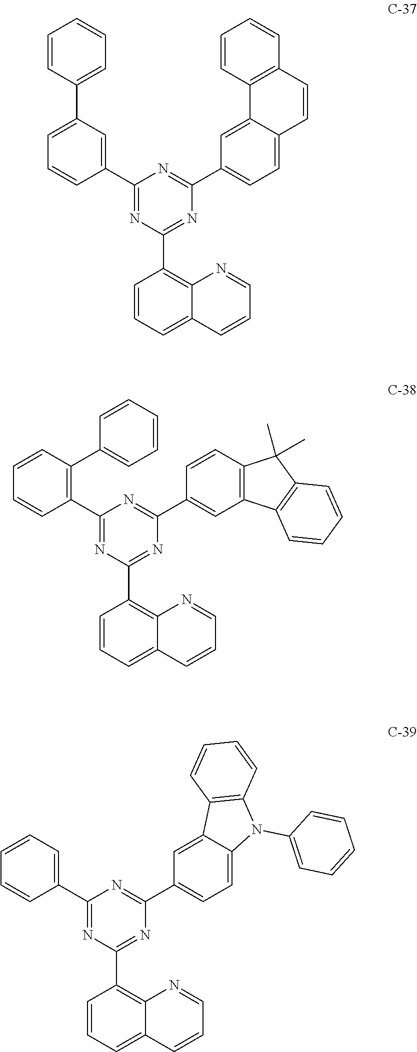 [SMILES: c1ccc(-c2cccc(-c3nc(-c4ccc5ccc6ccccc6c5c4)nc(-c4cccc5cccnc45)n3)c2)cc1 ; CC1(C)c2ccccc2-c2cc(-c3nc(-c4ccccc4-c4ccccc4)nc(-c4cccc5cccnc45)n3)ccc21 ; c1ccc(-c2nc(-c3ccc4c(c3)c3ccccc3n4-c3ccccc3)nc(-c3cccc4cccnc34)n2)cc1] 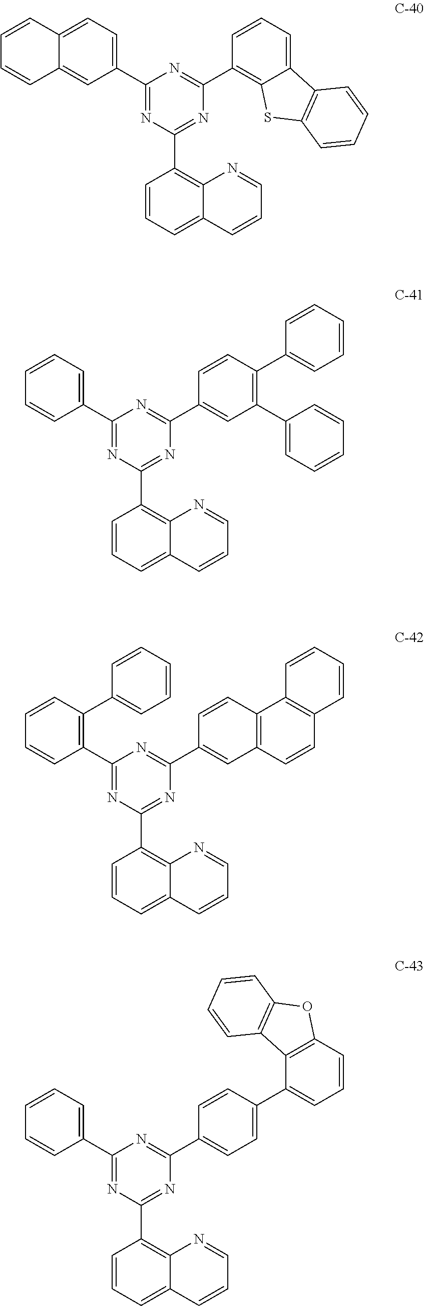 [SMILES: c1ccc2cc(-c3nc(-c4cccc5cccnc45)nc(-c4cccc5c4sc4ccccc45)n3)ccc2c1 ; c1ccc(-c2nc(-c3ccc(-c4ccccc4)c(-c4ccccc4)c3)nc(-c3cccc4cccnc34)n2)cc1 ; c1ccc(-c2ccccc2-c2nc(-c3ccc4c(ccc5ccccc54)c3)nc(-c3cccc4cccnc34)n2)cc1 ; c1ccc(-c2nc(-c3ccc(-c4cccc5oc6ccccc6c45)cc3)nc(-c3cccc4cccnc34)n2)cc1] 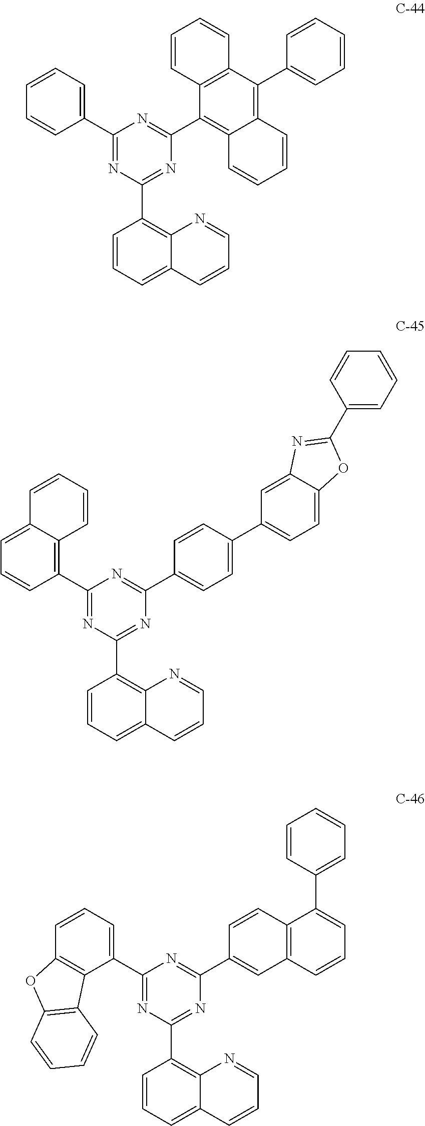 [SMILES: c1ccc(-c2nc(-c3c4ccccc4c(-c4ccccc4)c4ccccc34)nc(-c3cccc4cccnc34)n2)cc1 ; c1ccc(-c2nc3cc(-c4ccc(-c5nc(-c6cccc7ccccc67)nc(-c6cccc7cccnc67)n5)cc4)ccc3o2)cc1 ; c1ccc(-c2cccc3cc(-c4nc(-c5cccc6cccnc56)nc(-c5cccc6oc7ccccc7c56)n4)ccc23)cc1] 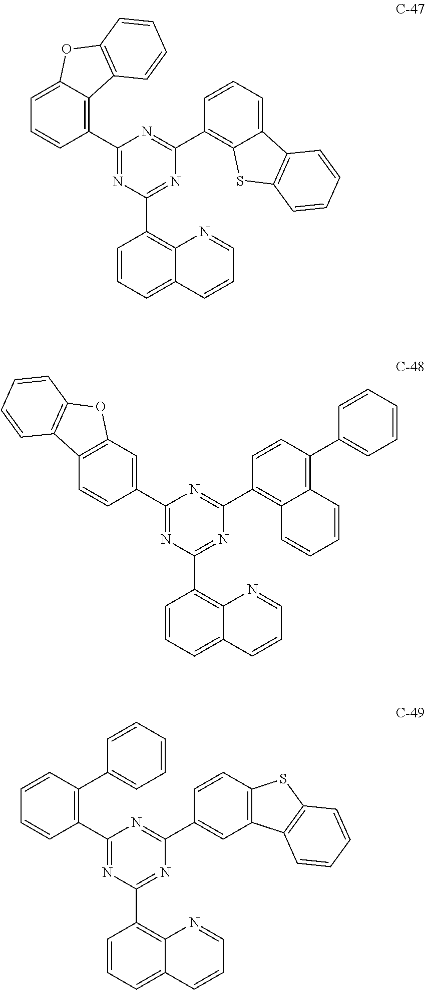 [SMILES: c1cnc2c(-c3nc(-c4cccc5c4sc4ccccc45)nc(-c4cccc5oc6ccccc6c45)n3)cccc2c1 ; c1ccc(-c2ccc(-c3nc(-c4ccc5c(c4)oc4ccccc45)nc(-c4cccc5cccnc45)n3)c3ccccc23)cc1 ; c1ccc(-c2ccccc2-c2nc(-c3ccc4sc5ccccc5c4c3)nc(-c3cccc4cccnc34)n2)cc1] 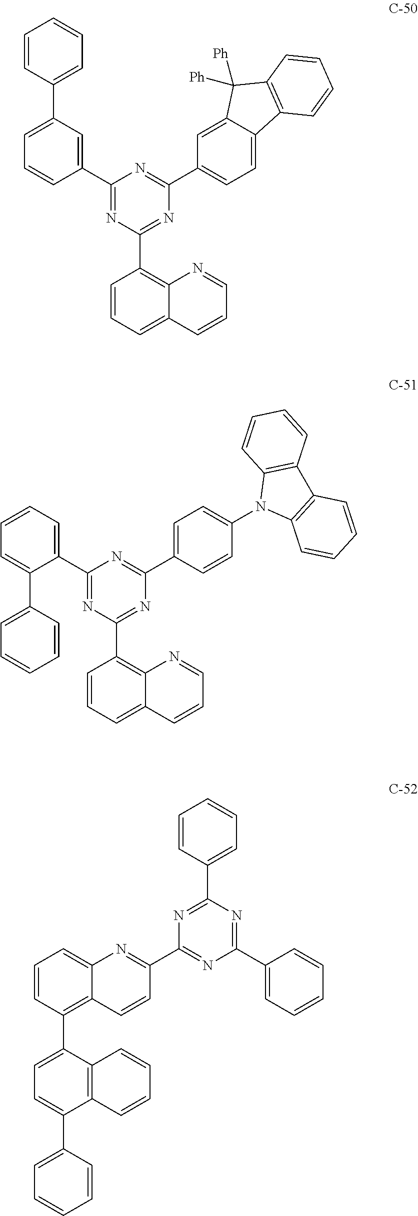 [SMILES: c1ccc(-c2cccc(-c3nc(-c4ccc5c(c4)C(c4ccccc4)(c4ccccc4)c4ccccc4-5)nc(-c4cccc5cccnc45)n3)c2)cc1 ; c1ccc(-c2ccccc2-c2nc(-c3ccc(-n4c5ccccc5c5ccccc54)cc3)nc(-c3cccc4cccnc34)n2)cc1 ; c1ccc(-c2nc(-c3ccccc3)nc(-c3ccc4c(-c5ccc(-c6ccccc6)c6ccccc56)cccc4n3)n2)cc1] 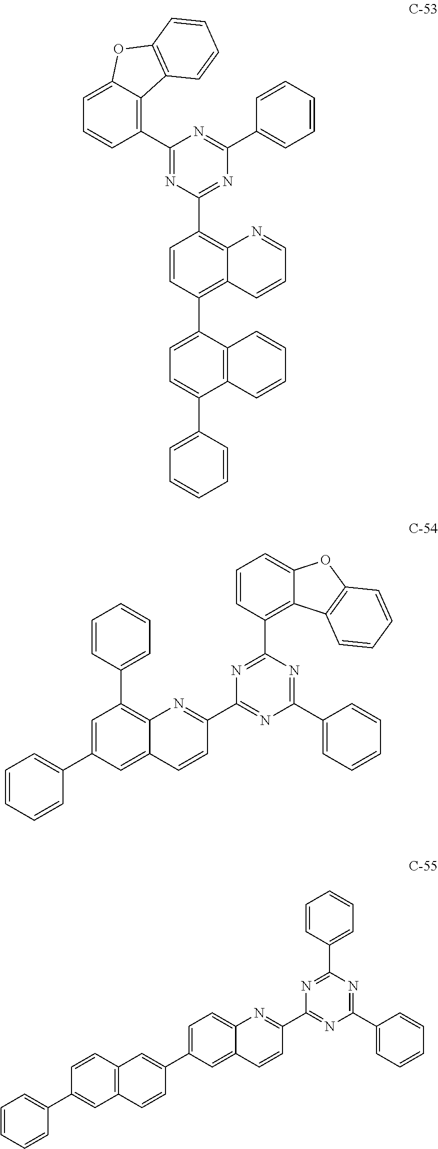 [SMILES: c1ccc(-c2nc(-c3ccc(-c4ccc(-c5ccccc5)c5ccccc45)c4cccnc34)nc(-c3cccc4oc5ccccc5c34)n2)cc1 ; c1ccc(-c2cc(-c3ccccc3)c3nc(-c4nc(-c5ccccc5)nc(-c5cccc6oc7ccccc7c56)n4)ccc3c2)cc1 ; c1ccc(-c2ccc3cc(-c4ccc5nc(-c6nc(-c7ccccc7)nc(-c7ccccc7)n6)ccc5c4)ccc3c2)cc1] 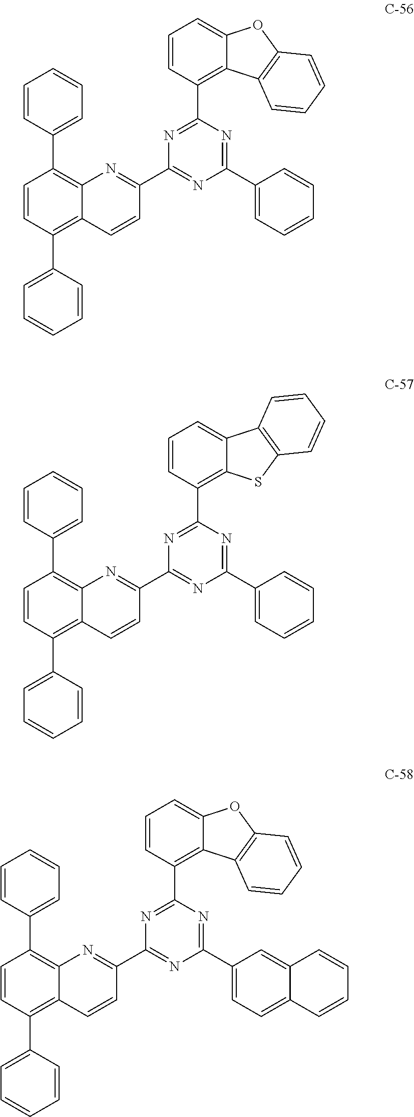 [SMILES: c1ccc(-c2nc(-c3ccc4c(-c5ccccc5)ccc(-c5ccccc5)c4n3)nc(-c3cccc4oc5ccccc5c34)n2)cc1 ; c1ccc(-c2nc(-c3ccc4c(-c5ccccc5)ccc(-c5ccccc5)c4n3)nc(-c3cccc4c3sc3ccccc34)n2)cc1 ; c1ccc(-c2ccc(-c3ccccc3)c3nc(-c4nc(-c5ccc6ccccc6c5)nc(-c5cccc6oc7ccccc7c56)n4)ccc23)cc1] 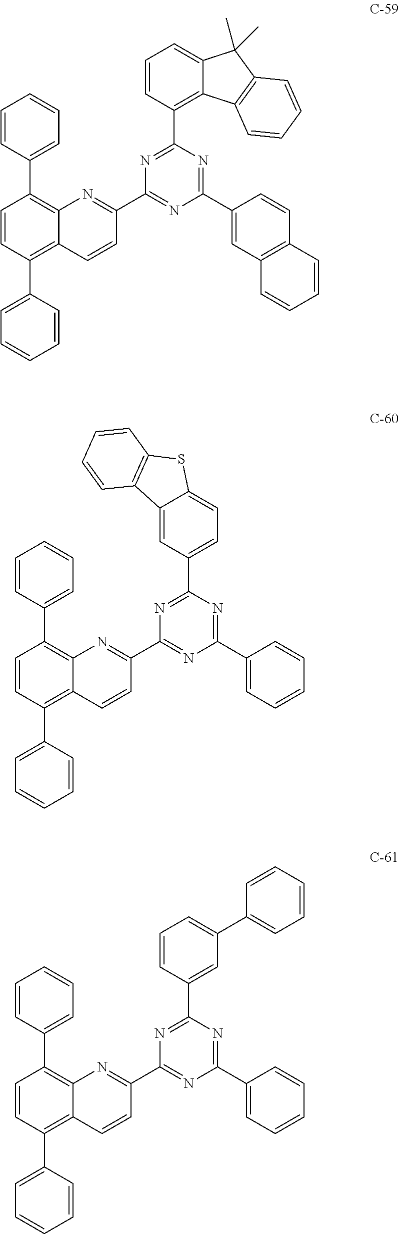 [SMILES: CC1(C)c2ccccc2-c2c(-c3nc(-c4ccc5ccccc5c4)nc(-c4ccc5c(-c6ccccc6)ccc(-c6ccccc6)c5n4)n3)cccc21 ; c1ccc(-c2nc(-c3ccc4sc5ccccc5c4c3)nc(-c3ccc4c(-c5ccccc5)ccc(-c5ccccc5)c4n3)n2)cc1 ; c1ccc(-c2cccc(-c3nc(-c4ccccc4)nc(-c4ccc5c(-c6ccccc6)ccc(-c6ccccc6)c5n4)n3)c2)cc1] 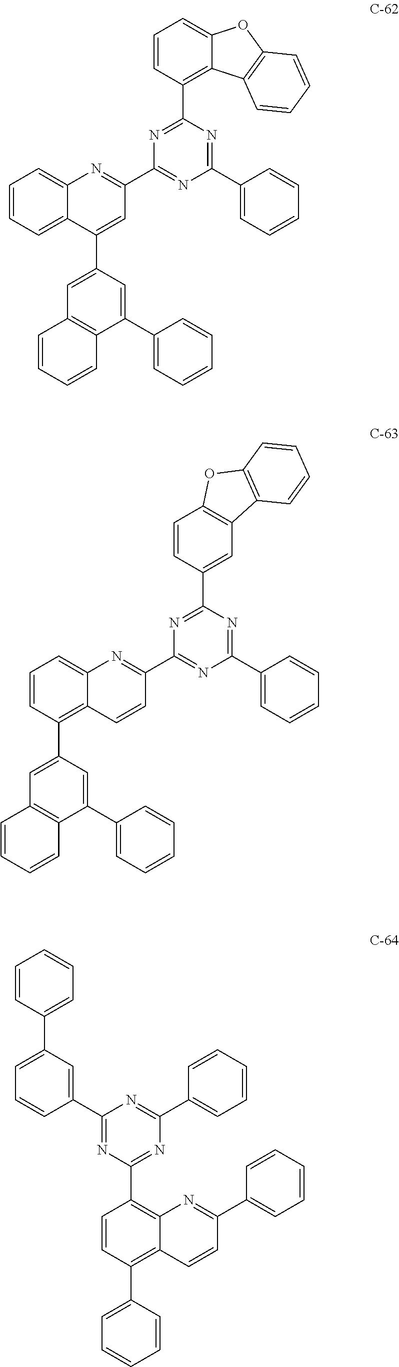 [SMILES: c1ccc(-c2nc(-c3cc(-c4cc(-c5ccccc5)c5ccccc5c4)c4ccccc4n3)nc(-c3cccc4oc5ccccc5c34)n2)cc1 ; c1ccc(-c2nc(-c3ccc4oc5ccccc5c4c3)nc(-c3ccc4c(-c5cc(-c6ccccc6)c6ccccc6c5)cccc4n3)n2)cc1 ; c1ccc(-c2cccc(-c3nc(-c4ccccc4)nc(-c4ccc(-c5ccccc5)c5ccc(-c6ccccc6)nc45)n3)c2)cc1] 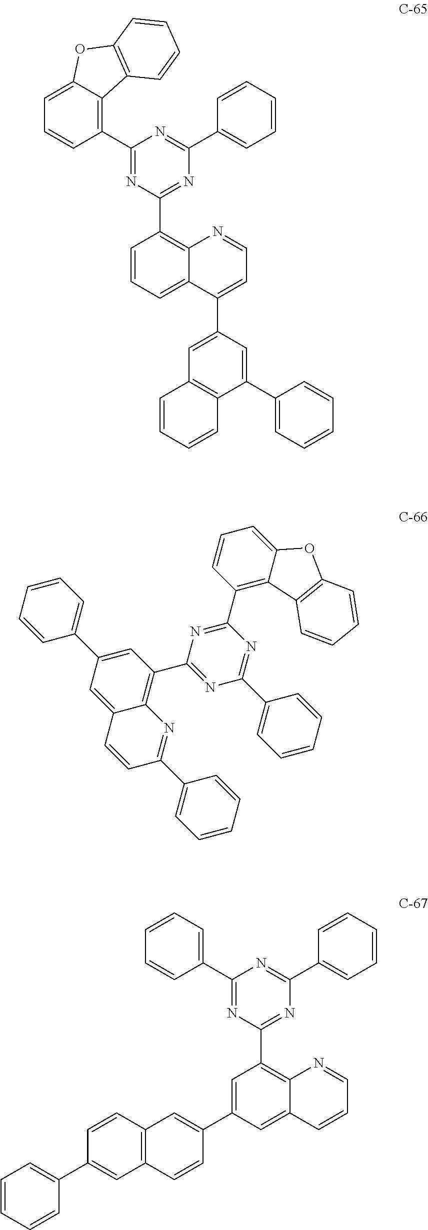 [SMILES: c1ccc(-c2nc(-c3cccc4c(-c5cc(-c6ccccc6)c6ccccc6c5)ccnc34)nc(-c3cccc4oc5ccccc5c34)n2)cc1 ; c1ccc(-c2cc(-c3nc(-c4ccccc4)nc(-c4cccc5oc6ccccc6c45)n3)c3nc(-c4ccccc4)ccc3c2)cc1 ; c1ccc(-c2ccc3cc(-c4cc(-c5nc(-c6ccccc6)nc(-c6ccccc6)n5)c5ncccc5c4)ccc3c2)cc1] 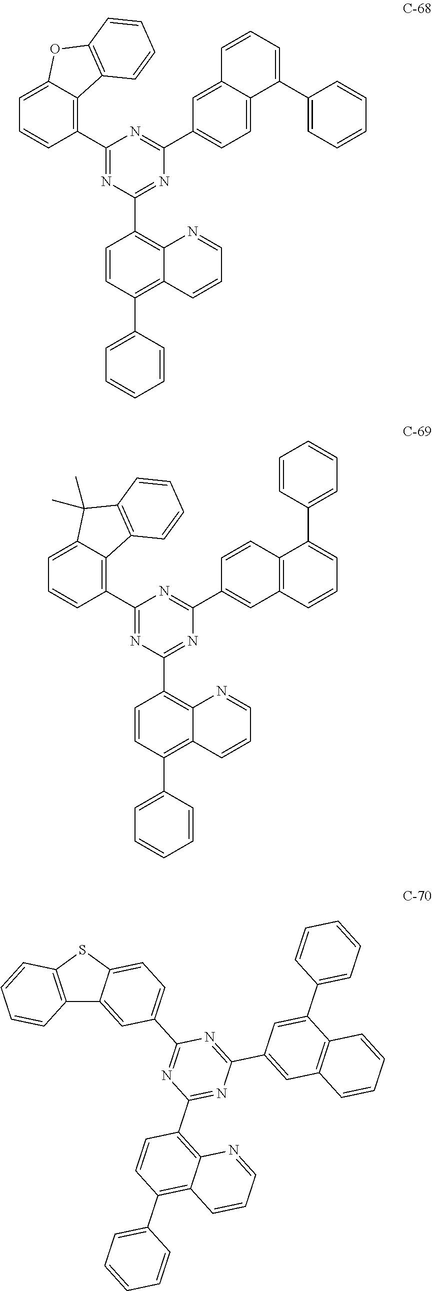 [SMILES: c1ccc(-c2cccc3cc(-c4nc(-c5ccc(-c6ccccc6)c6cccnc56)nc(-c5cccc6oc7ccccc7c56)n4)ccc23)cc1 ; CC1(C)c2ccccc2-c2c(-c3nc(-c4ccc5c(-c6ccccc6)cccc5c4)nc(-c4ccc(-c5ccccc5)c5cccnc45)n3)cccc21 ; c1ccc(-c2cc(-c3nc(-c4ccc5sc6ccccc6c5c4)nc(-c4ccc(-c5ccccc5)c5cccnc45)n3)cc3ccccc23)cc1] 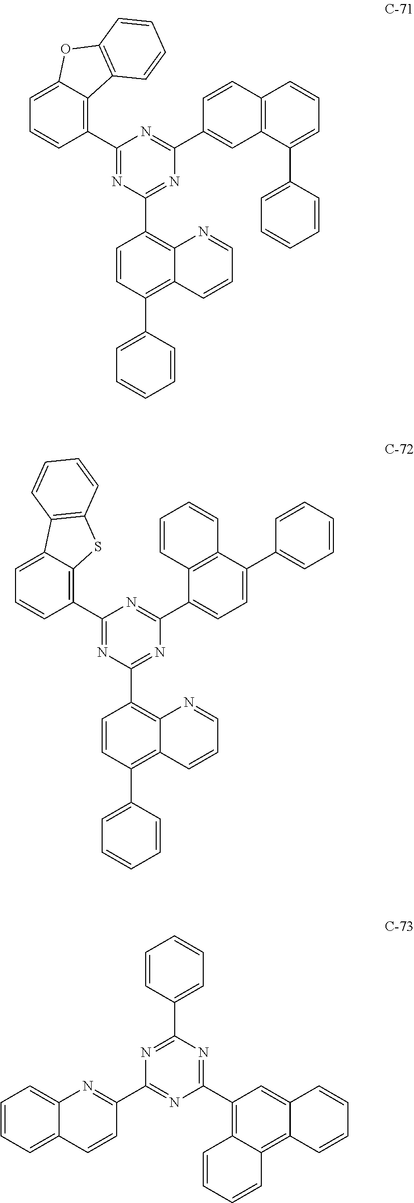 [SMILES: c1ccc(-c2cccc3ccc(-c4nc(-c5ccc(-c6ccccc6)c6cccnc56)nc(-c5cccc6oc7ccccc7c56)n4)cc23)cc1 ; c1ccc(-c2ccc(-c3nc(-c4ccc(-c5ccccc5)c5cccnc45)nc(-c4cccc5c4sc4ccccc45)n3)c3ccccc23)cc1 ; c1ccc(-c2nc(-c3ccc4ccccc4n3)nc(-c3cc4ccccc4c4ccccc34)n2)cc1] 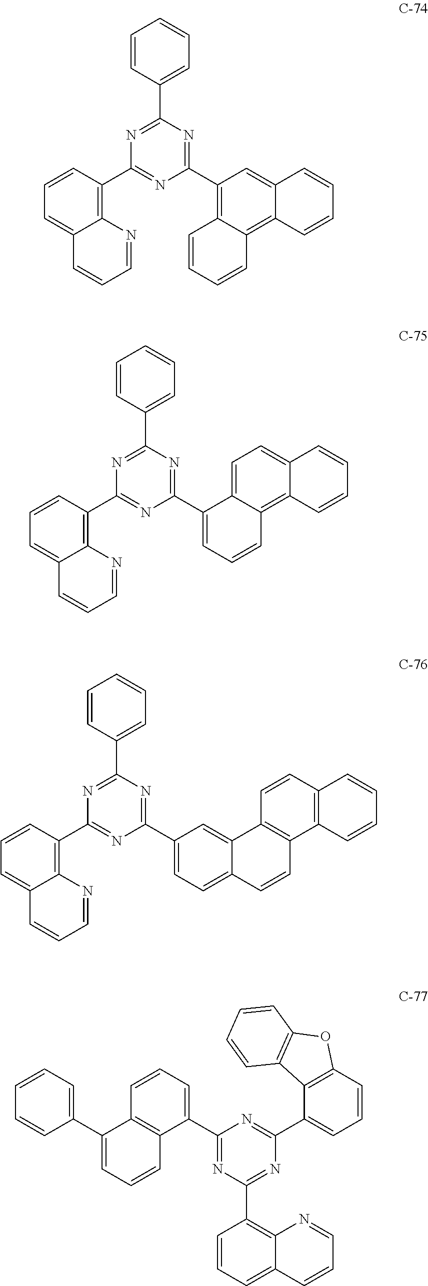 [SMILES: c1ccc(-c2nc(-c3cc4ccccc4c4ccccc34)nc(-c3cccc4cccnc34)n2)cc1 ; c1ccc(-c2nc(-c3cccc4c3ccc3ccccc34)nc(-c3cccc4cccnc34)n2)cc1 ; c1ccc(-c2nc(-c3ccc4ccc5c6ccccc6ccc5c4c3)nc(-c3cccc4cccnc34)n2)cc1 ; c1ccc(-c2cccc3c(-c4nc(-c5cccc6cccnc56)nc(-c5cccc6oc7ccccc7c56)n4)cccc23)cc1] 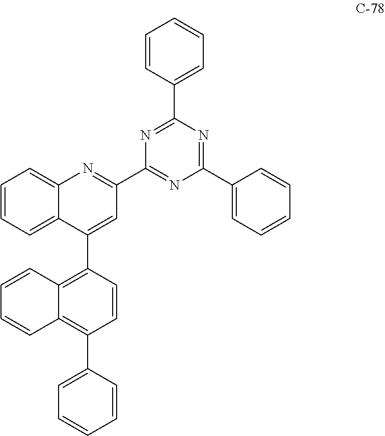 [SMILES: c1ccc(-c2nc(-c3ccccc3)nc(-c3cc(-c4ccc(-c5ccccc5)c5ccccc45)c4ccccc4n3)n2)cc1] 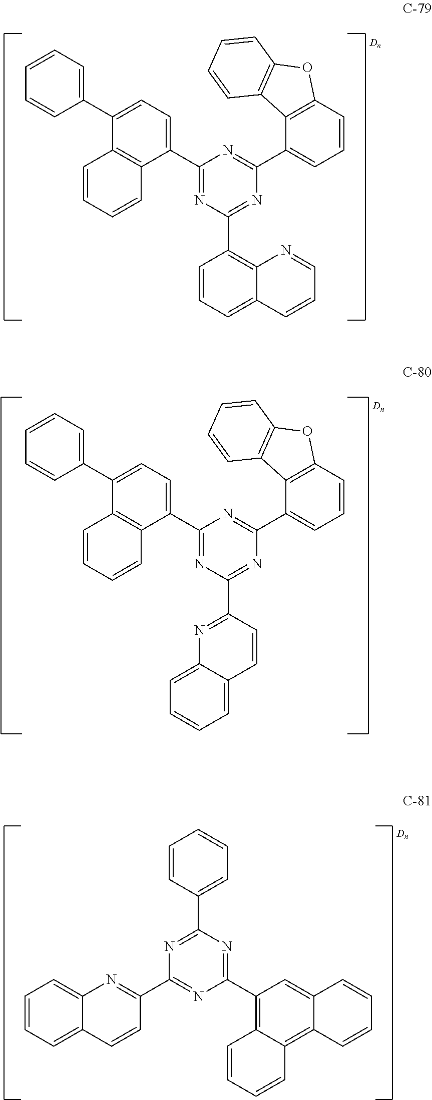 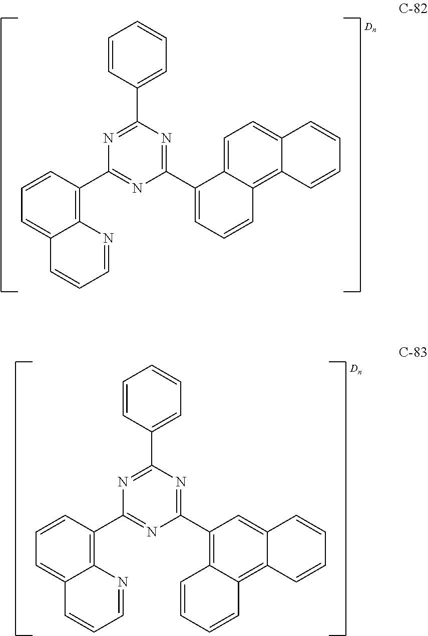 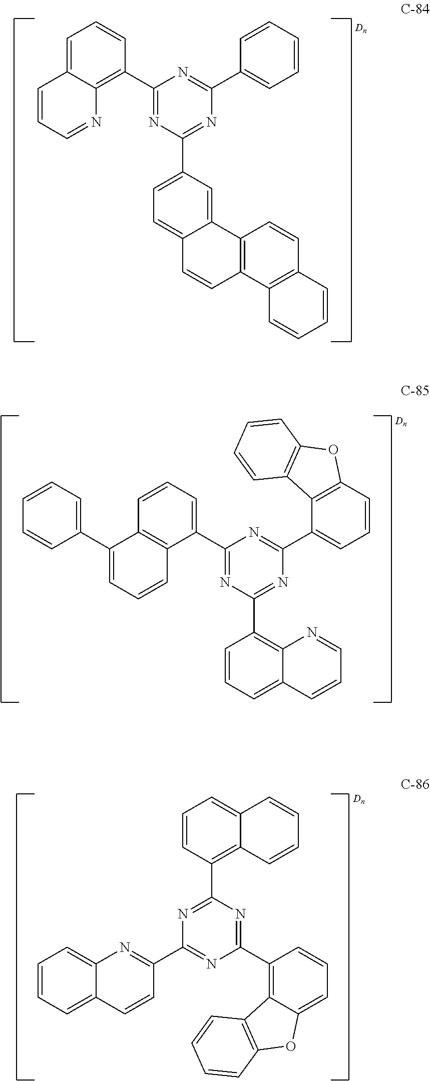 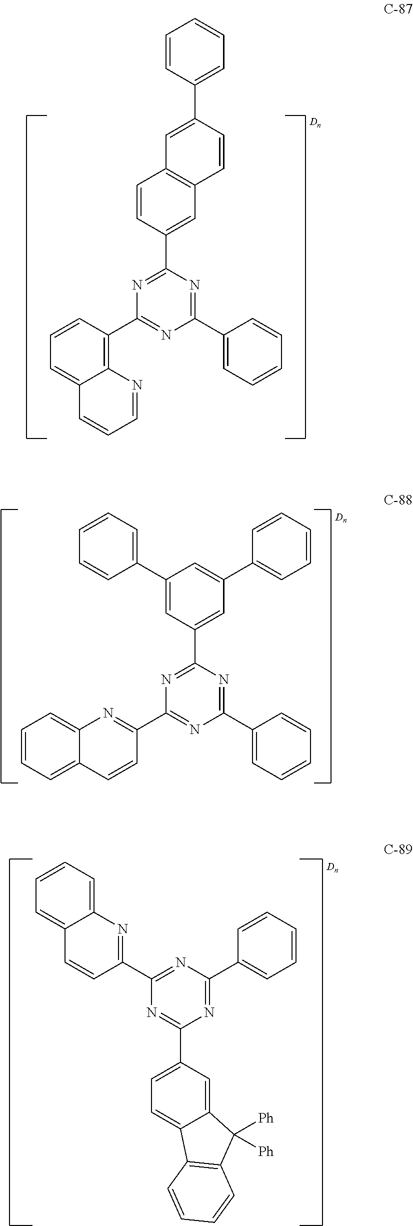 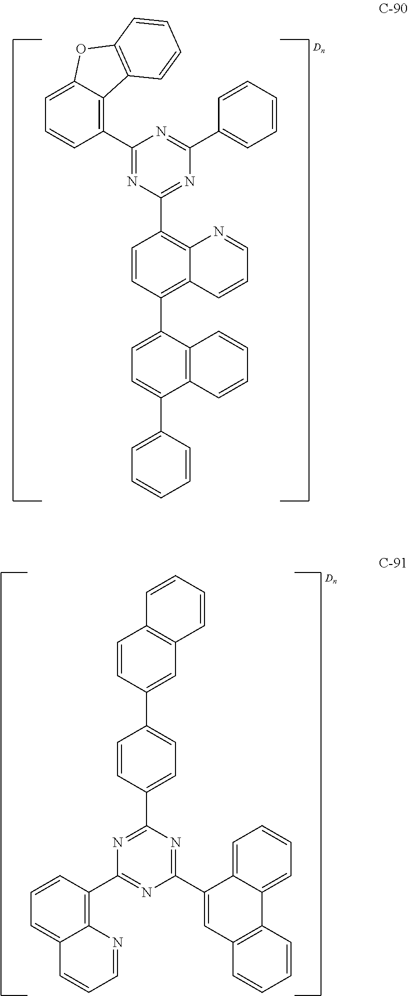 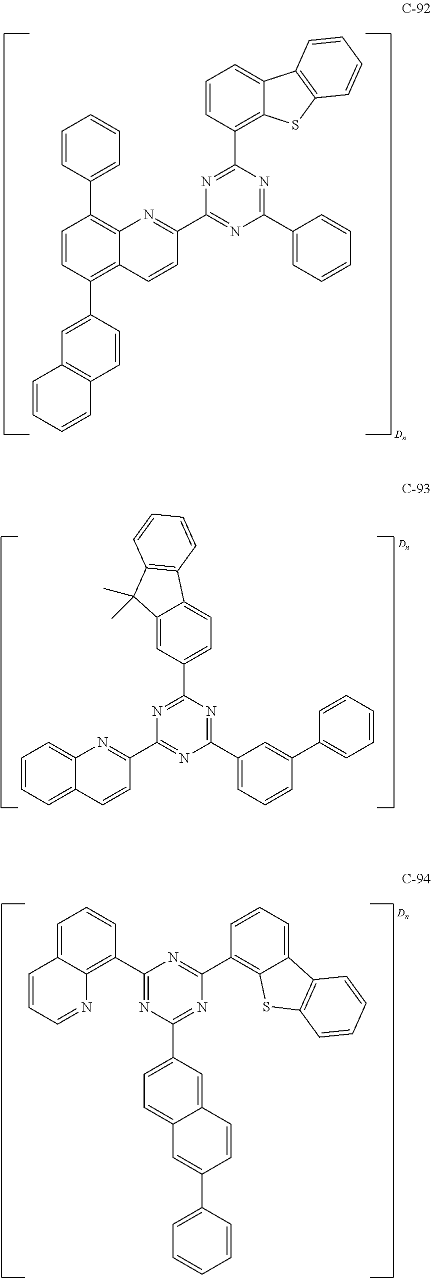 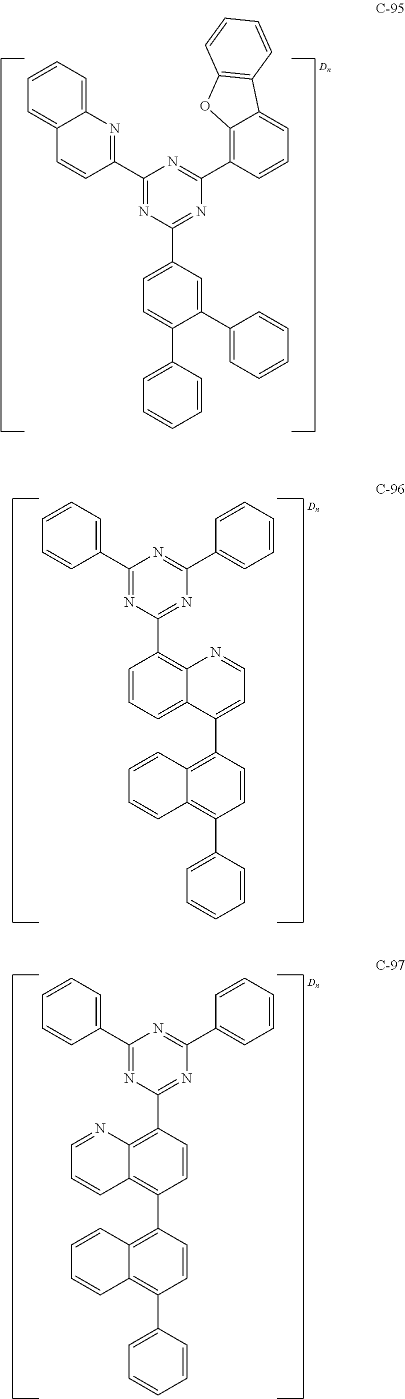 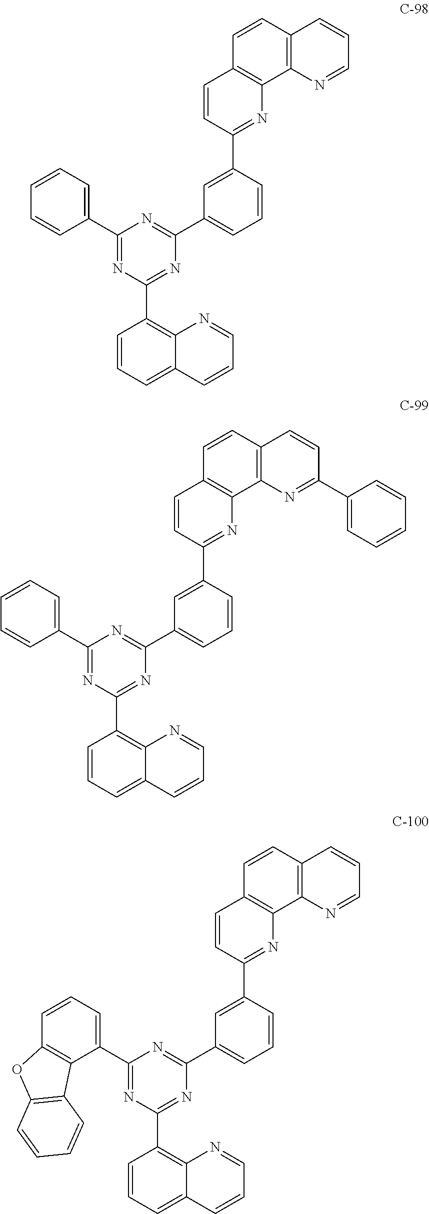 [SMILES: c1ccc(-c2nc(-c3cccc(-c4ccc5ccc6cccnc6c5n4)c3)nc(-c3cccc4cccnc34)n2)cc1 ; c1ccc(-c2ccc3ccc4ccc(-c5cccc(-c6nc(-c7ccccc7)nc(-c7cccc8cccnc78)n6)c5)nc4c3n2)cc1 ; c1cc(-c2ccc3ccc4cccnc4c3n2)cc(-c2nc(-c3cccc4cccnc34)nc(-c3cccc4oc5ccccc5c34)n2)c1] 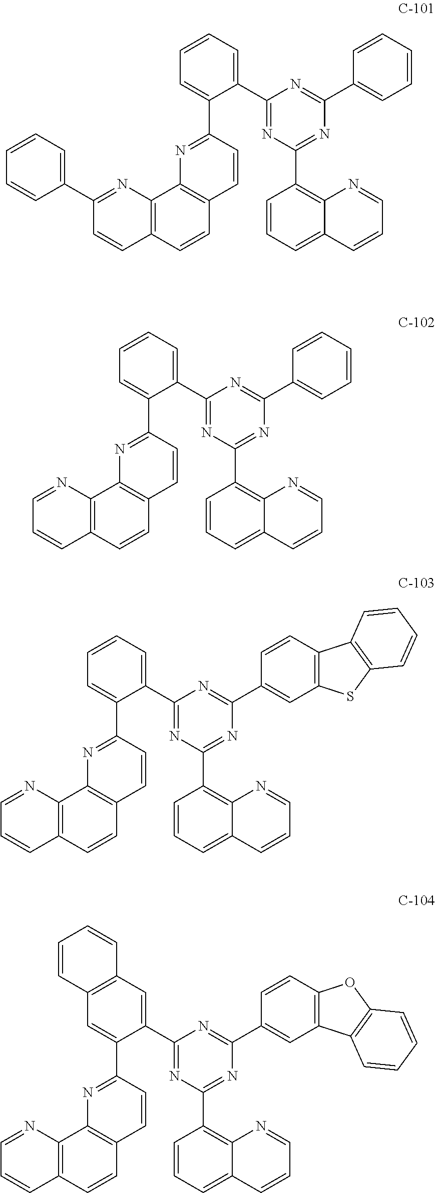 [SMILES: c1ccc(-c2ccc3ccc4ccc(-c5ccccc5-c5nc(-c6ccccc6)nc(-c6cccc7cccnc67)n5)nc4c3n2)cc1 ; c1ccc(-c2nc(-c3ccccc3-c3ccc4ccc5cccnc5c4n3)nc(-c3cccc4cccnc34)n2)cc1 ; c1ccc(-c2nc(-c3ccc4c(c3)sc3ccccc34)nc(-c3cccc4cccnc34)n2)c(-c2ccc3ccc4cccnc4c3n2)c1 ; c1ccc2cc(-c3nc(-c4ccc5oc6ccccc6c5c4)nc(-c4cccc5cccnc45)n3)c(-c3ccc4ccc5cccnc5c4n3)cc2c1] 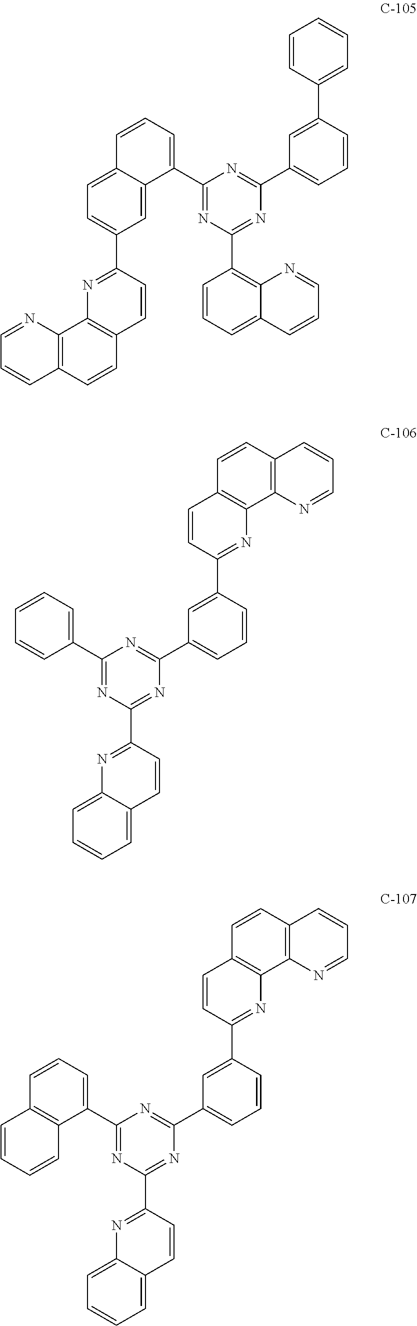 [SMILES: c1ccc(-c2cccc(-c3nc(-c4cccc5ccc(-c6ccc7ccc8cccnc8c7n6)cc45)nc(-c4cccc5cccnc45)n3)c2)cc1 ; c1ccc(-c2nc(-c3cccc(-c4ccc5ccc6cccnc6c5n4)c3)nc(-c3ccc4ccccc4n3)n2)cc1 ; c1cc(-c2ccc3ccc4cccnc4c3n2)cc(-c2nc(-c3ccc4ccccc4n3)nc(-c3cccc4ccccc34)n2)c1] 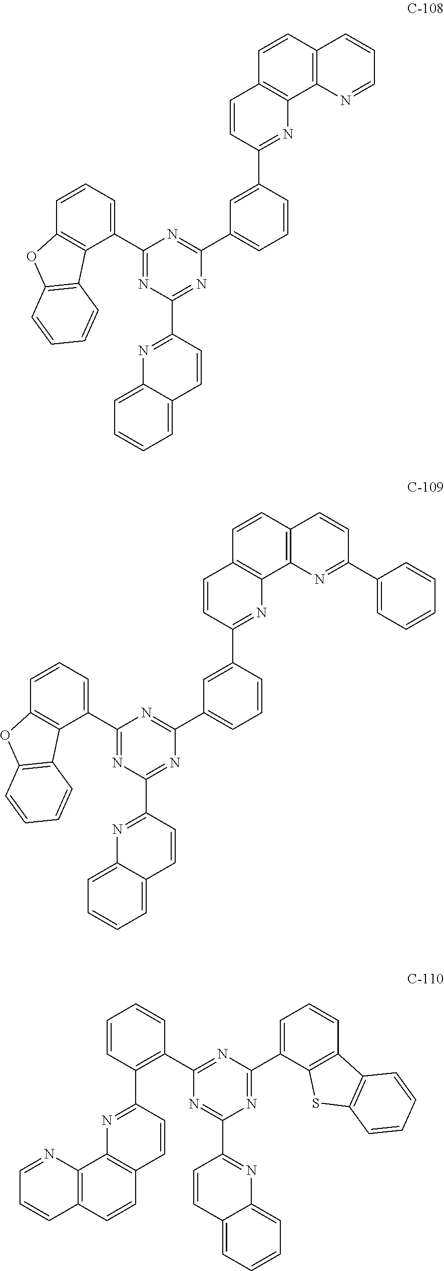 [SMILES: c1cc(-c2ccc3ccc4cccnc4c3n2)cc(-c2nc(-c3ccc4ccccc4n3)nc(-c3cccc4oc5ccccc5c34)n2)c1 ; c1ccc(-c2ccc3ccc4ccc(-c5cccc(-c6nc(-c7ccc8ccccc8n7)nc(-c7cccc8oc9ccccc9c78)n6)c5)nc4c3n2)cc1 ; c1ccc(-c2nc(-c3ccc4ccccc4n3)nc(-c3cccc4c3sc3ccccc34)n2)c(-c2ccc3ccc4cccnc4c3n2)c1] 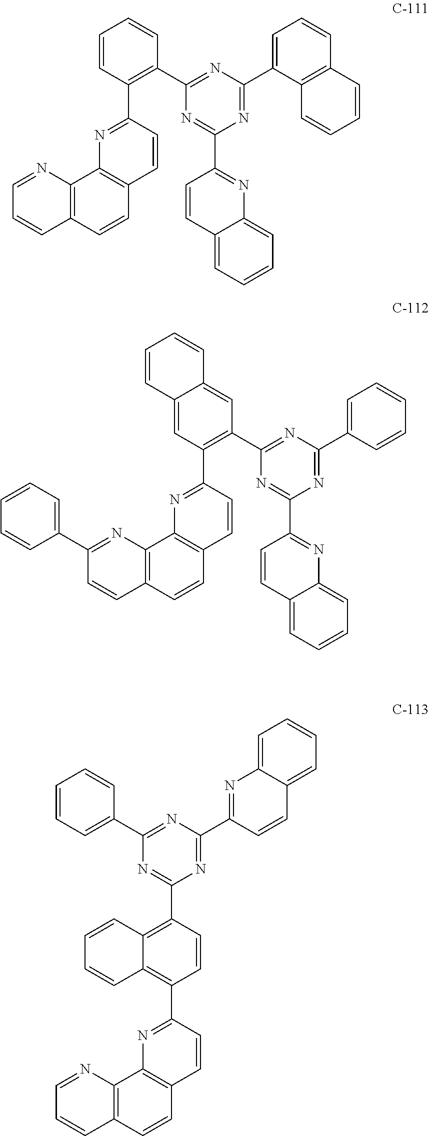 [SMILES: c1ccc(-c2nc(-c3ccc4ccccc4n3)nc(-c3cccc4ccccc34)n2)c(-c2ccc3ccc4cccnc4c3n2)c1 ; c1ccc(-c2ccc3ccc4ccc(-c5cc6ccccc6cc5-c5nc(-c6ccccc6)nc(-c6ccc7ccccc7n6)n5)nc4c3n2)cc1 ; c1ccc(-c2nc(-c3ccc4ccccc4n3)nc(-c3ccc(-c4ccc5ccc6cccnc6c5n4)c4ccccc34)n2)cc1] 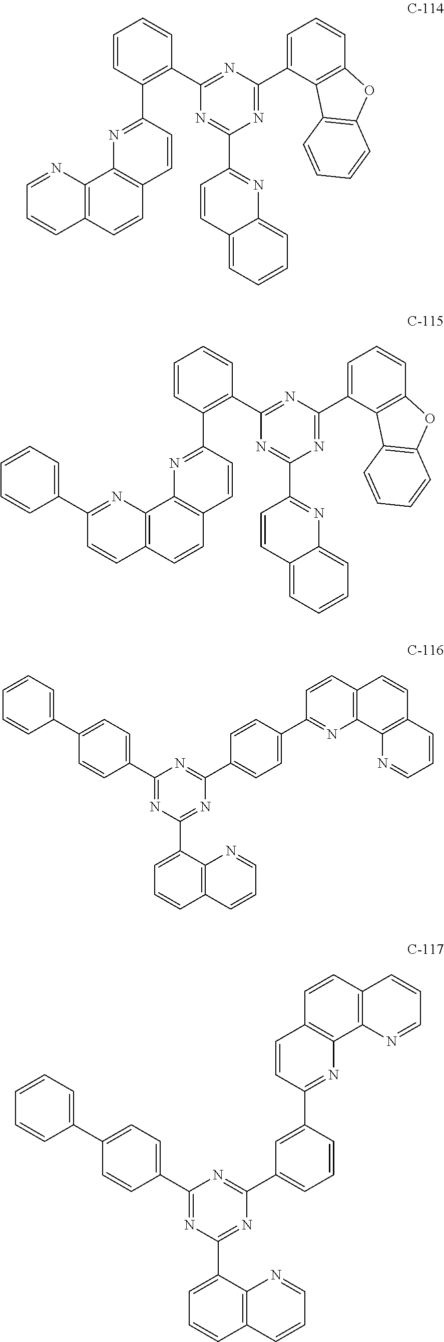 [SMILES: c1ccc(-c2nc(-c3ccc4ccccc4n3)nc(-c3cccc4oc5ccccc5c34)n2)c(-c2ccc3ccc4cccnc4c3n2)c1 ; c1ccc(-c2ccc3ccc4ccc(-c5ccccc5-c5nc(-c6ccc7ccccc7n6)nc(-c6cccc7oc8ccccc8c67)n5)nc4c3n2)cc1 ; c1ccc(-c2ccc(-c3nc(-c4ccc(-c5ccc6ccc7cccnc7c6n5)cc4)nc(-c4cccc5cccnc45)n3)cc2)cc1 ; c1ccc(-c2ccc(-c3nc(-c4cccc(-c5ccc6ccc7cccnc7c6n5)c4)nc(-c4cccc5cccnc45)n3)cc2)cc1] 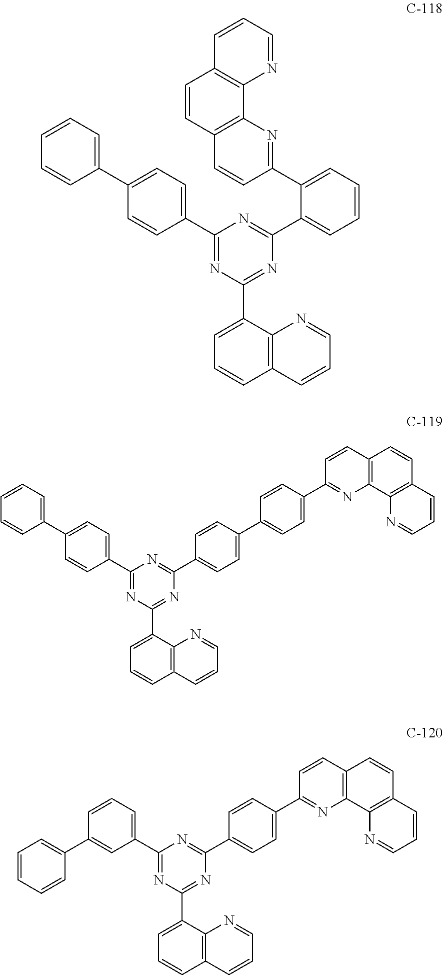 [SMILES: c1ccc(-c2ccc(-c3nc(-c4ccccc4-c4ccc5ccc6cccnc6c5n4)nc(-c4cccc5cccnc45)n3)cc2)cc1 ; c1ccc(-c2ccc(-c3nc(-c4ccc(-c5ccc(-c6ccc7ccc8cccnc8c7n6)cc5)cc4)nc(-c4cccc5cccnc45)n3)cc2)cc1 ; c1ccc(-c2cccc(-c3nc(-c4ccc(-c5ccc6ccc7cccnc7c6n5)cc4)nc(-c4cccc5cccnc45)n3)c2)cc1] 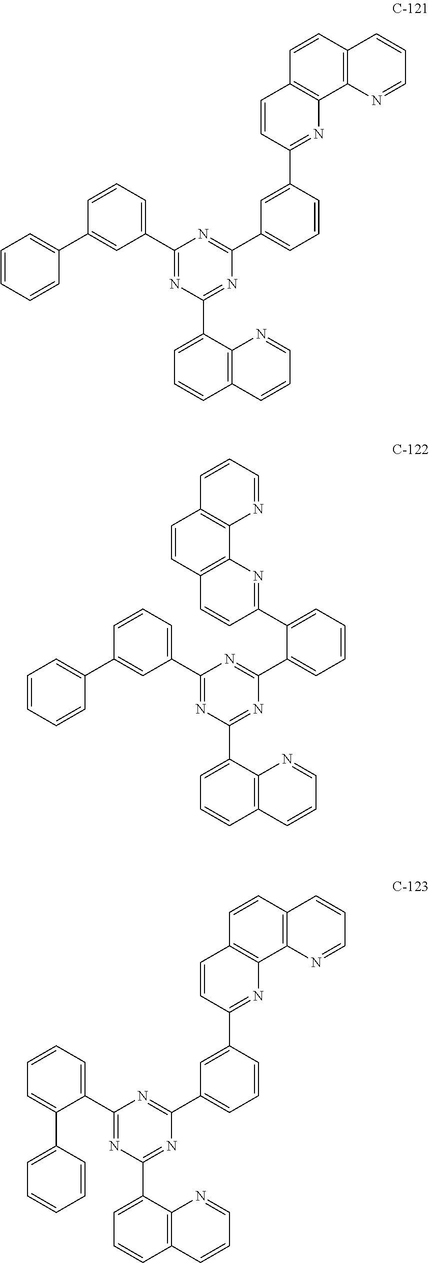 [SMILES: c1ccc(-c2cccc(-c3nc(-c4cccc(-c5ccc6ccc7cccnc7c6n5)c4)nc(-c4cccc5cccnc45)n3)c2)cc1 ; c1ccc(-c2cccc(-c3nc(-c4ccccc4-c4ccc5ccc6cccnc6c5n4)nc(-c4cccc5cccnc45)n3)c2)cc1 ; c1ccc(-c2ccccc2-c2nc(-c3cccc(-c4ccc5ccc6cccnc6c5n4)c3)nc(-c3cccc4cccnc34)n2)cc1] 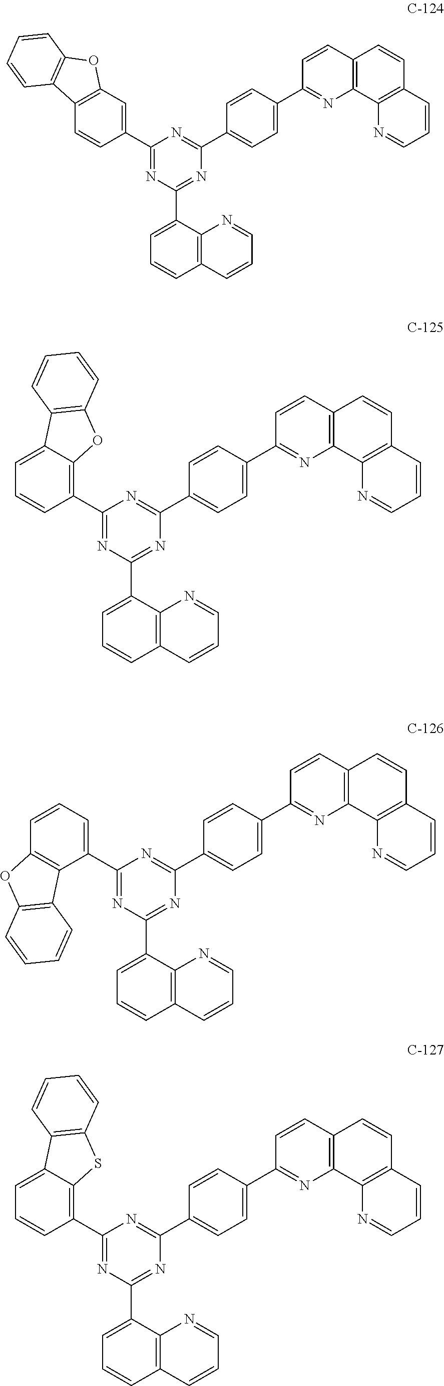 [SMILES: c1cnc2c(-c3nc(-c4ccc(-c5ccc6ccc7cccnc7c6n5)cc4)nc(-c4ccc5c(c4)oc4ccccc45)n3)cccc2c1 ; c1cnc2c(-c3nc(-c4ccc(-c5ccc6ccc7cccnc7c6n5)cc4)nc(-c4cccc5c4oc4ccccc45)n3)cccc2c1 ; c1cnc2c(-c3nc(-c4ccc(-c5ccc6ccc7cccnc7c6n5)cc4)nc(-c4cccc5oc6ccccc6c45)n3)cccc2c1 ; c1cnc2c(-c3nc(-c4ccc(-c5ccc6ccc7cccnc7c6n5)cc4)nc(-c4cccc5c4sc4ccccc45)n3)cccc2c1] 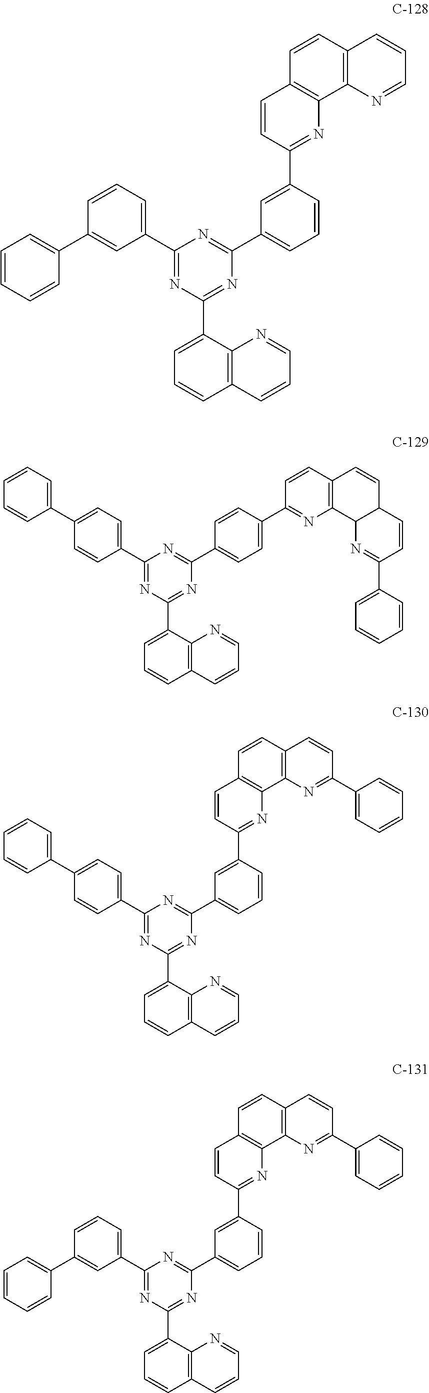 [SMILES: c1ccc(-c2cccc(-c3nc(-c4cccc(-c5ccc6ccc7cccnc7c6n5)c4)nc(-c4cccc5cccnc45)n3)c2)cc1 ; C1=CC2C=Cc3ccc(-c4ccc(-c5nc(-c6ccc(-c7ccccc7)cc6)nc(-c6cccc7cccnc67)n5)cc4)nc3C2N=C1c1ccccc1 ; c1ccc(-c2ccc(-c3nc(-c4cccc(-c5ccc6ccc7ccc(-c8ccccc8)nc7c6n5)c4)nc(-c4cccc5cccnc45)n3)cc2)cc1 ; c1ccc(-c2cccc(-c3nc(-c4cccc(-c5ccc6ccc7ccc(-c8ccccc8)nc7c6n5)c4)nc(-c4cccc5cccnc45)n3)c2)cc1] 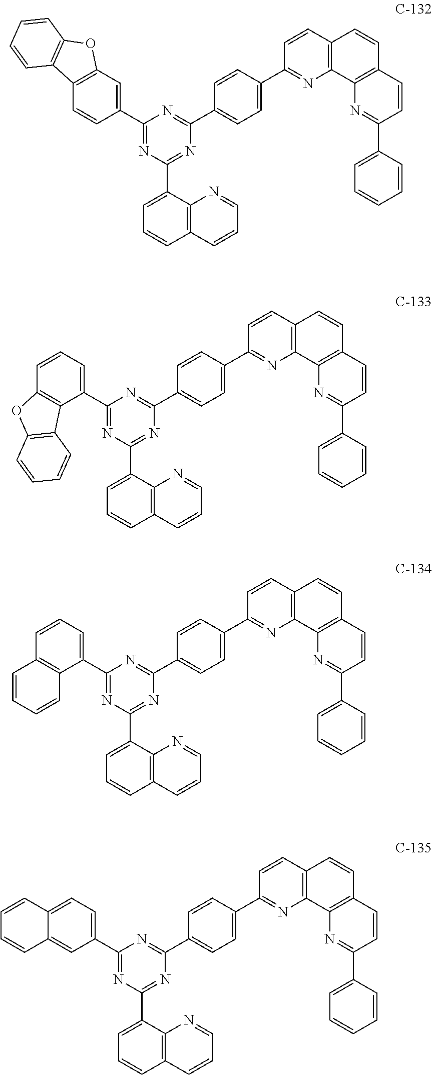 [SMILES: c1ccc(-c2ccc3ccc4ccc(-c5ccc(-c6nc(-c7ccc8c(c7)oc7ccccc78)nc(-c7cccc8cccnc78)n6)cc5)nc4c3n2)cc1 ; c1ccc(-c2ccc3ccc4ccc(-c5ccc(-c6nc(-c7cccc8cccnc78)nc(-c7cccc8oc9ccccc9c78)n6)cc5)nc4c3n2)cc1 ; c1ccc(-c2ccc3ccc4ccc(-c5ccc(-c6nc(-c7cccc8ccccc78)nc(-c7cccc8cccnc78)n6)cc5)nc4c3n2)cc1 ; c1ccc(-c2ccc3ccc4ccc(-c5ccc(-c6nc(-c7ccc8ccccc8c7)nc(-c7cccc8cccnc78)n6)cc5)nc4c3n2)cc1] 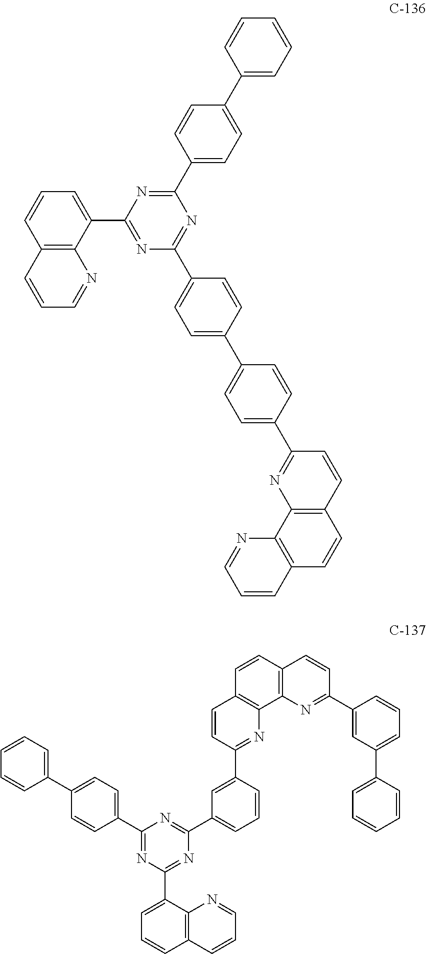 [SMILES: c1ccc(-c2ccc(-c3nc(-c4ccc(-c5ccc(-c6ccc7ccc8cccnc8c7n6)cc5)cc4)nc(-c4cccc5cccnc45)n3)cc2)cc1 ; c1ccc(-c2ccc(-c3nc(-c4cccc(-c5ccc6ccc7ccc(-c8cccc(-c9ccccc9)c8)nc7c6n5)c4)nc(-c4cccc5cccnc45)n3)cc2)cc1] 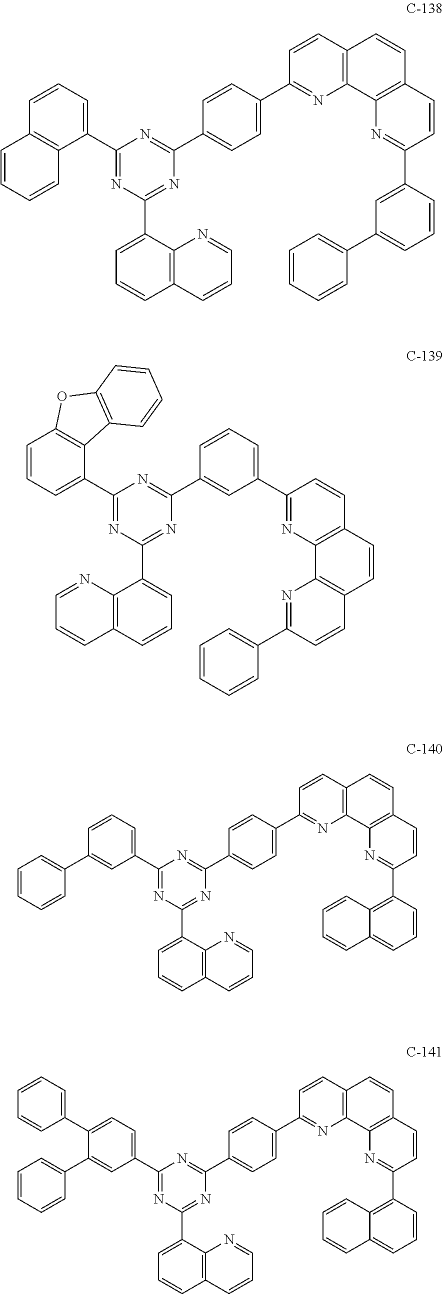 [SMILES: c1ccc(-c2cccc(-c3ccc4ccc5ccc(-c6ccc(-c7nc(-c8cccc9ccccc89)nc(-c8cccc9cccnc89)n7)cc6)nc5c4n3)c2)cc1 ; c1ccc(-c2ccc3ccc4ccc(-c5cccc(-c6nc(-c7cccc8cccnc78)nc(-c7cccc8oc9ccccc9c78)n6)c5)nc4c3n2)cc1 ; c1ccc(-c2cccc(-c3nc(-c4ccc(-c5ccc6ccc7ccc(-c8cccc9ccccc89)nc7c6n5)cc4)nc(-c4cccc5cccnc45)n3)c2)cc1 ; c1ccc(-c2ccc(-c3nc(-c4ccc(-c5ccc6ccc7ccc(-c8cccc9ccccc89)nc7c6n5)cc4)nc(-c4cccc5cccnc45)n3)cc2-c2ccccc2)cc1] 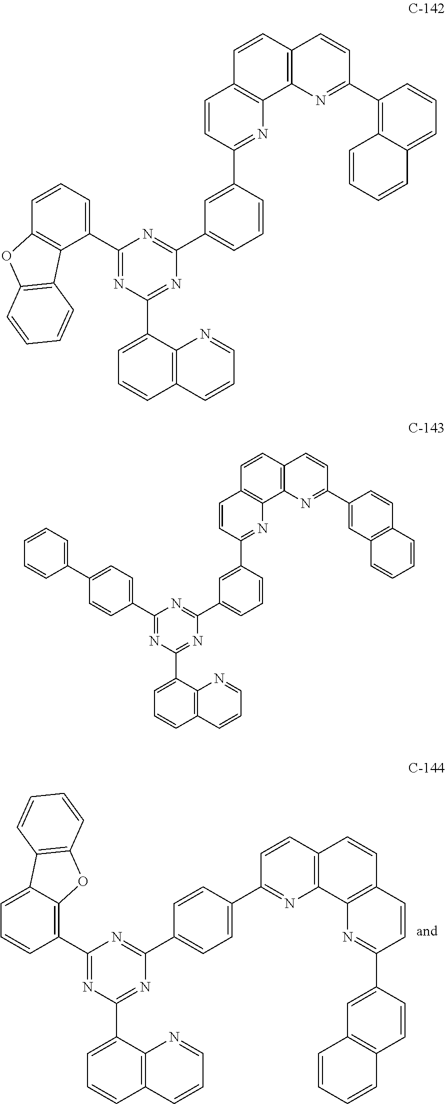 [SMILES: c1cc(-c2ccc3ccc4ccc(-c5cccc6ccccc56)nc4c3n2)cc(-c2nc(-c3cccc4cccnc34)nc(-c3cccc4oc5ccccc5c34)n2)c1 ; c1ccc(-c2ccc(-c3nc(-c4cccc(-c5ccc6ccc7ccc(-c8ccc9ccccc9c8)nc7c6n5)c4)nc(-c4cccc5cccnc45)n3)cc2)cc1 ; c1ccc2cc(-c3ccc4ccc5ccc(-c6ccc(-c7nc(-c8cccc9cccnc89)nc(-c8cccc9c8oc8ccccc89)n7)cc6)nc5c4n3)ccc2c1] 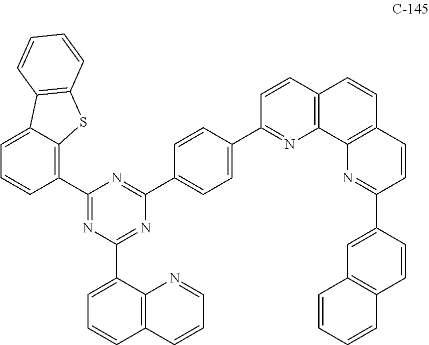 [SMILES: c1ccc2cc(-c3ccc4ccc5ccc(-c6ccc(-c7nc(-c8cccc9cccnc89)nc(-c8cccc9c8sc8ccccc89)n7)cc6)nc5c4n3)ccc2c1]
wherein “Dₙ” in the above compounds means that n number of hydrogens is replaced by deuterium, wherein n is from 1 to the maximum number of hydrogen in the compound.

7. An organic electroluminescent compound represented by the following formula 3: 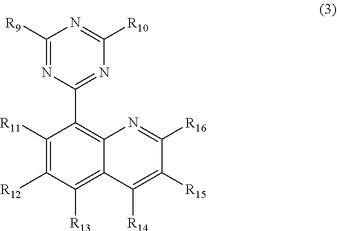
In formula 3,
R₉ to R₁₆ each independently represent, hydrogen, deuterium, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, or a substituted or unsubstituted tri(C6-C30)arylsilyl; and
With the proviso that neither R₉ nor R₁₀ is a substituted or unsubstituted fluorenyl.

8. The organic electroluminescence device according to claim 7, wherein the compound represented by formula 3 is selected from the following compounds: 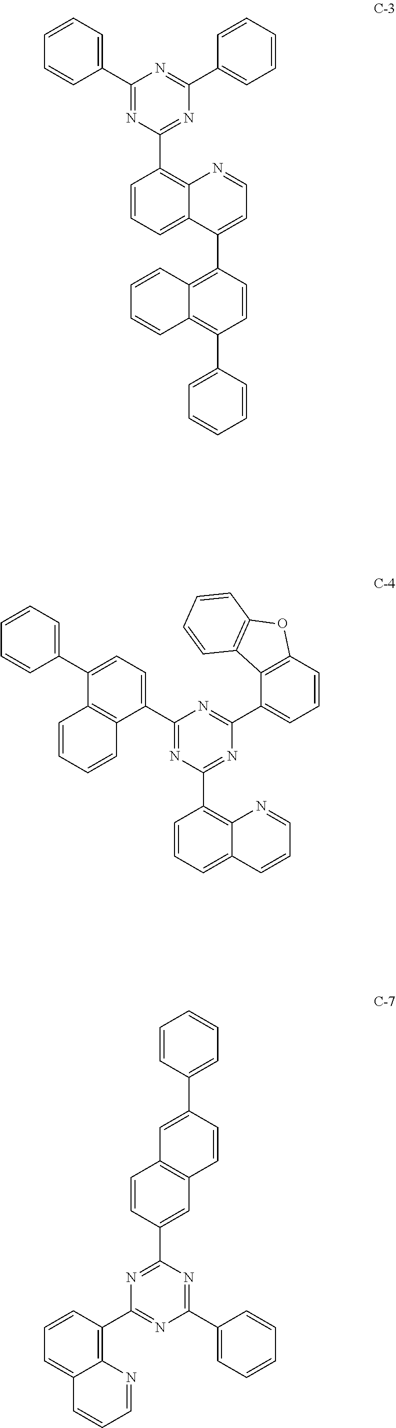 [SMILES: c1ccc(-c2nc(-c3ccccc3)nc(-c3cccc4c(-c5ccc(-c6ccccc6)c6ccccc56)ccnc34)n2)cc1 ; c1ccc(-c2ccc(-c3nc(-c4cccc5cccnc45)nc(-c4cccc5oc6ccccc6c45)n3)c3ccccc23)cc1 ; c1ccc(-c2ccc3cc(-c4nc(-c5ccccc5)nc(-c5cccc6cccnc56)n4)ccc3c2)cc1] 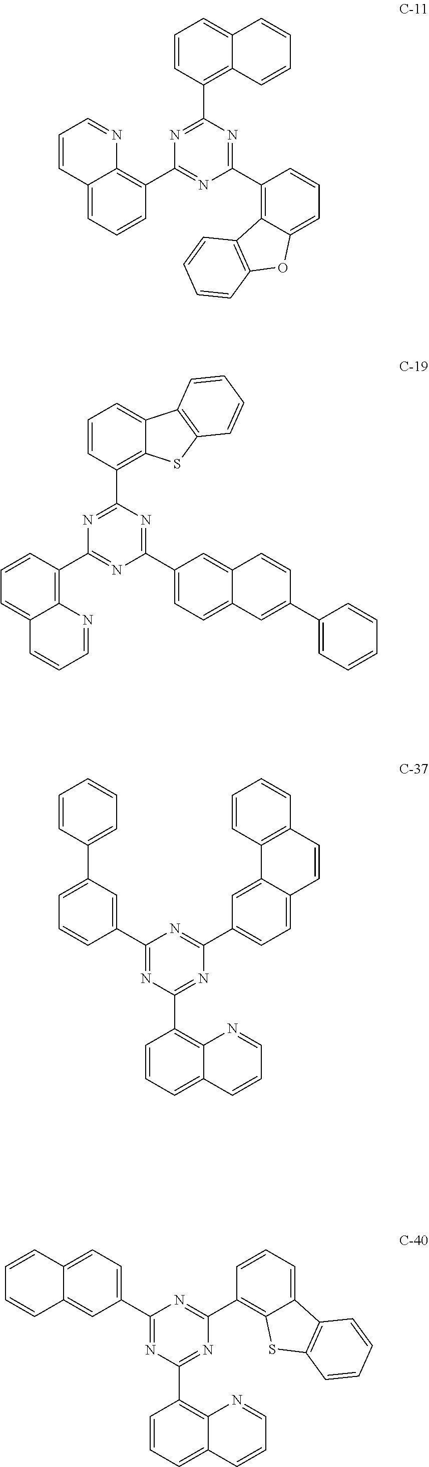 [SMILES: c1ccc2c(-c3nc(-c4cccc5cccnc45)nc(-c4cccc5oc6ccccc6c45)n3)cccc2c1 ; c1ccc(-c2ccc3cc(-c4nc(-c5cccc6cccnc56)nc(-c5cccc6c5sc5ccccc56)n4)ccc3c2)cc1 ; c1ccc(-c2cccc(-c3nc(-c4ccc5ccc6ccccc6c5c4)nc(-c4cccc5cccnc45)n3)c2)cc1 ; c1ccc2cc(-c3nc(-c4cccc5cccnc45)nc(-c4cccc5c4sc4ccccc45)n3)ccc2c1] 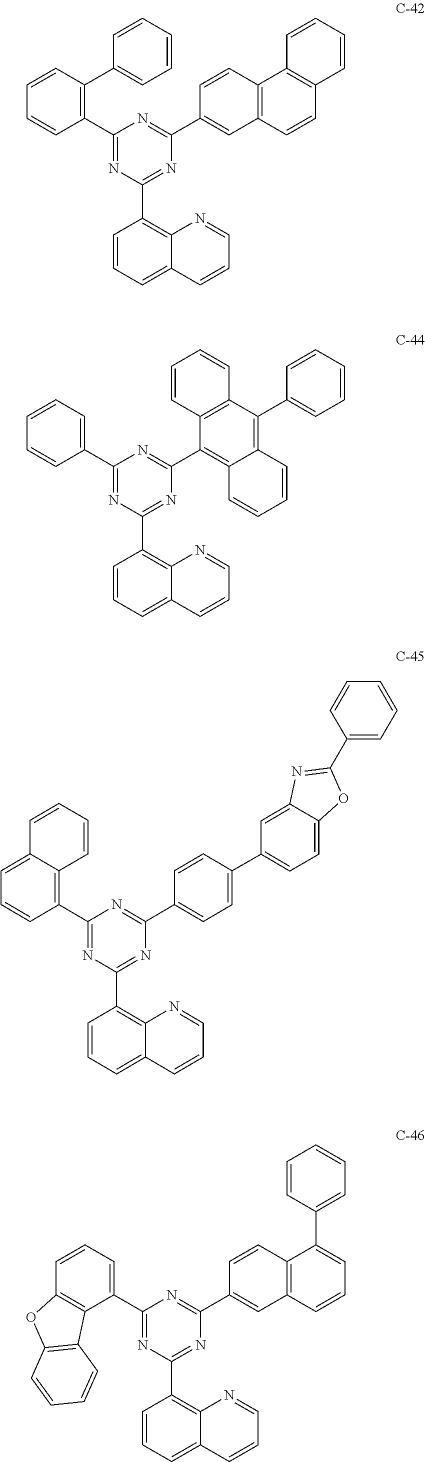 [SMILES: c1ccc(-c2ccccc2-c2nc(-c3ccc4c(ccc5ccccc54)c3)nc(-c3cccc4cccnc34)n2)cc1 ; c1ccc(-c2nc(-c3c4ccccc4c(-c4ccccc4)c4ccccc34)nc(-c3cccc4cccnc34)n2)cc1 ; c1ccc(-c2nc3cc(-c4ccc(-c5nc(-c6cccc7ccccc67)nc(-c6cccc7cccnc67)n5)cc4)ccc3o2)cc1 ; c1ccc(-c2cccc3cc(-c4nc(-c5cccc6cccnc56)nc(-c5cccc6oc7ccccc7c56)n4)ccc23)cc1] 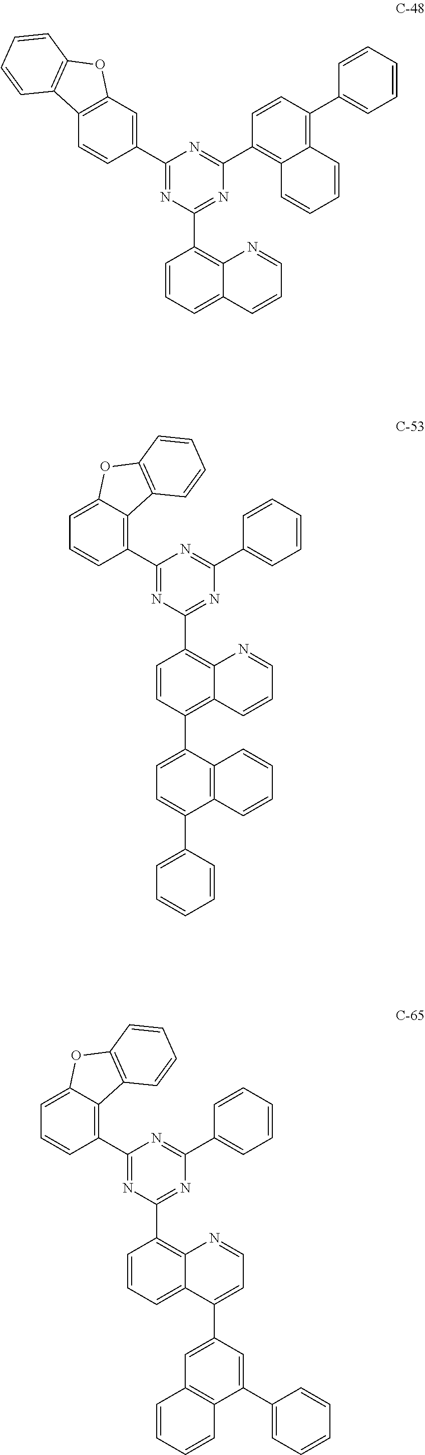 [SMILES: c1ccc(-c2ccc(-c3nc(-c4ccc5c(c4)oc4ccccc45)nc(-c4cccc5cccnc45)n3)c3ccccc23)cc1 ; c1ccc(-c2nc(-c3ccc(-c4ccc(-c5ccccc5)c5ccccc45)c4cccnc34)nc(-c3cccc4oc5ccccc5c34)n2)cc1 ; c1ccc(-c2nc(-c3cccc4c(-c5cc(-c6ccccc6)c6ccccc6c5)ccnc34)nc(-c3cccc4oc5ccccc5c34)n2)cc1] 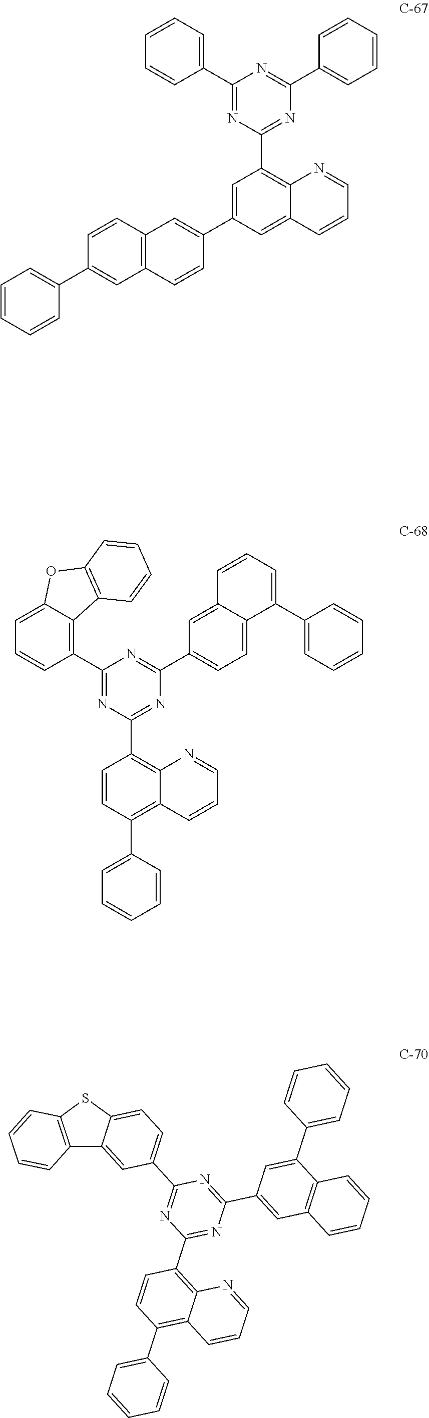 [SMILES: c1ccc(-c2ccc3cc(-c4cc(-c5nc(-c6ccccc6)nc(-c6ccccc6)n5)c5ncccc5c4)ccc3c2)cc1 ; c1ccc(-c2cccc3cc(-c4nc(-c5ccc(-c6ccccc6)c6cccnc56)nc(-c5cccc6oc7ccccc7c56)n4)ccc23)cc1 ; c1ccc(-c2cc(-c3nc(-c4ccc5sc6ccccc6c5c4)nc(-c4ccc(-c5ccccc5)c5cccnc45)n3)cc3ccccc23)cc1] 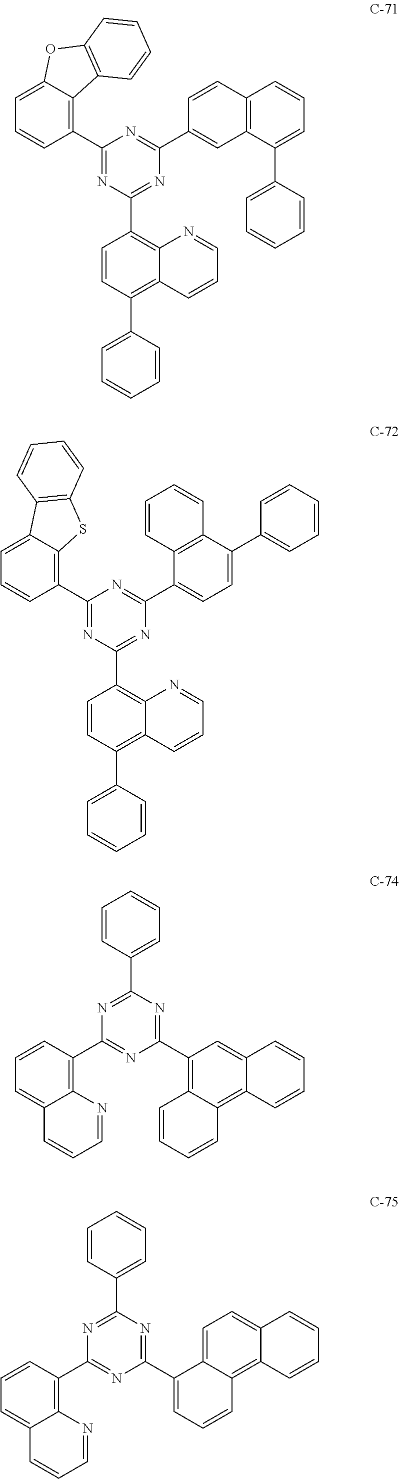 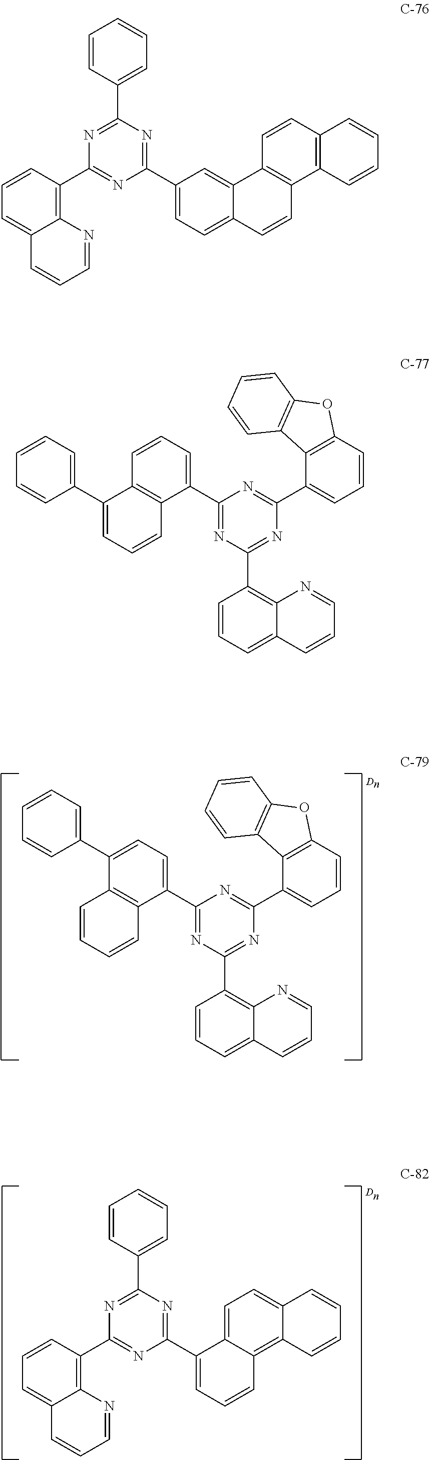 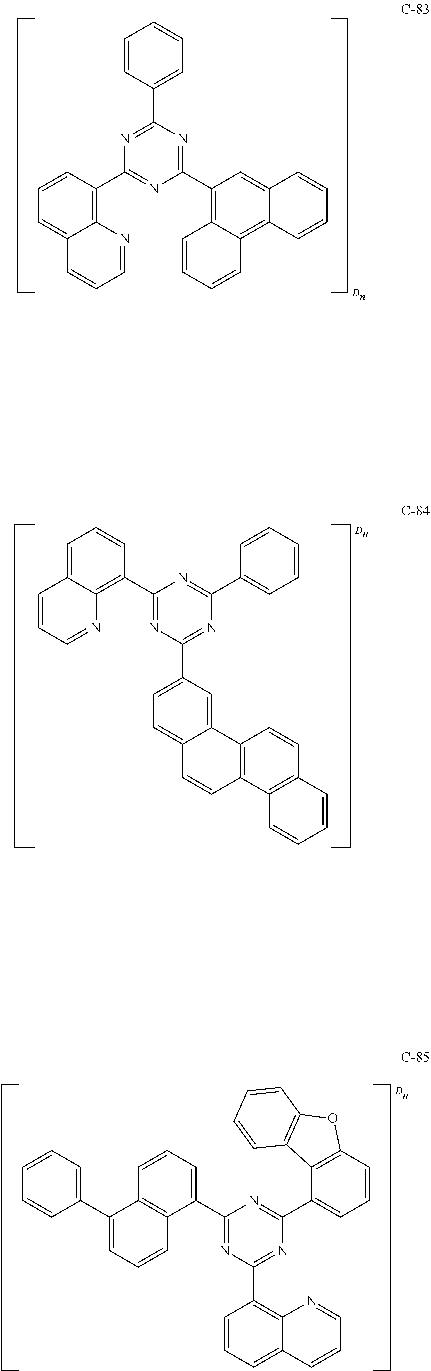 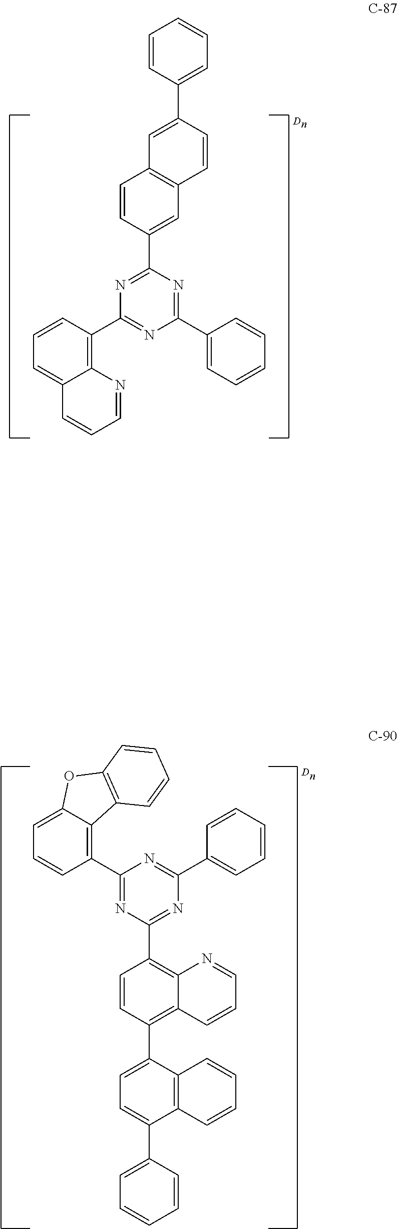 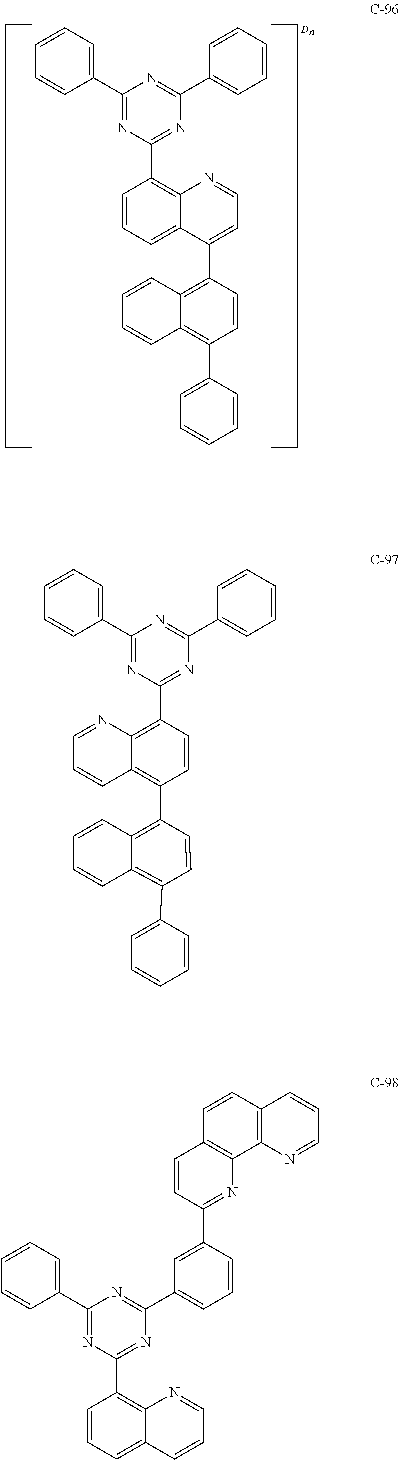 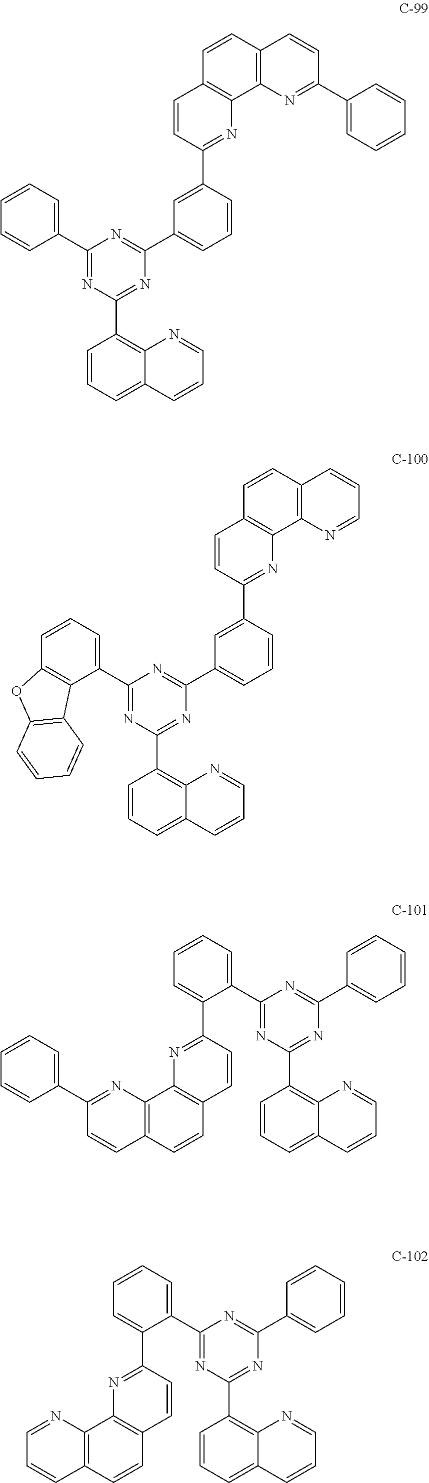 [SMILES: c1ccc(-c2ccc3ccc4ccc(-c5cccc(-c6nc(-c7ccccc7)nc(-c7cccc8cccnc78)n6)c5)nc4c3n2)cc1 ; c1cc(-c2ccc3ccc4cccnc4c3n2)cc(-c2nc(-c3cccc4cccnc34)nc(-c3cccc4oc5ccccc5c34)n2)c1 ; c1ccc(-c2ccc3ccc4ccc(-c5ccccc5-c5nc(-c6ccccc6)nc(-c6cccc7cccnc67)n5)nc4c3n2)cc1 ; c1ccc(-c2nc(-c3ccccc3-c3ccc4ccc5cccnc5c4n3)nc(-c3cccc4cccnc34)n2)cc1] 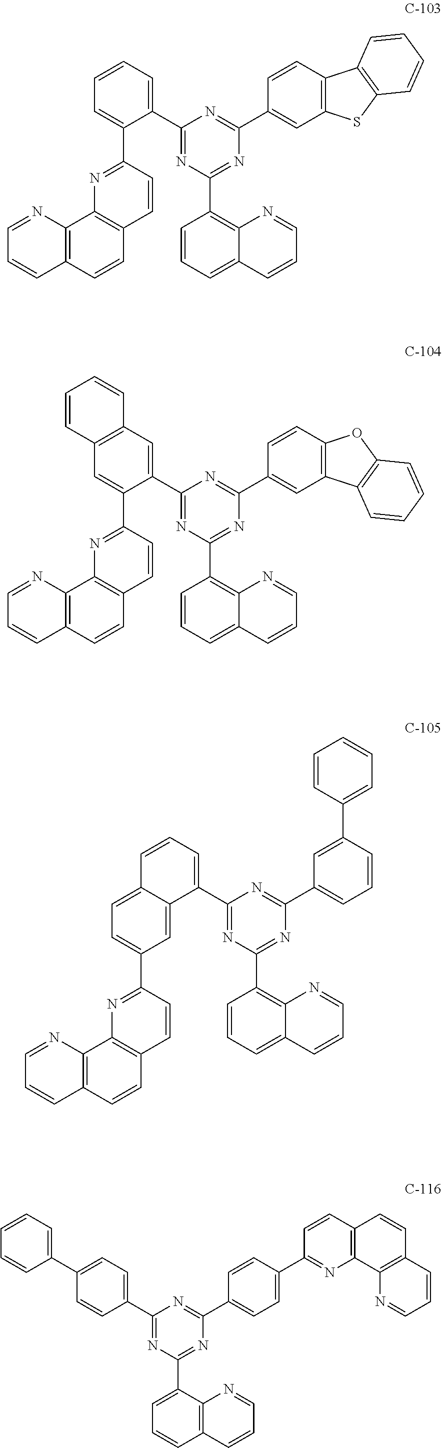 [SMILES: c1ccc(-c2nc(-c3ccc4c(c3)sc3ccccc34)nc(-c3cccc4cccnc34)n2)c(-c2ccc3ccc4cccnc4c3n2)c1 ; c1ccc2cc(-c3nc(-c4ccc5oc6ccccc6c5c4)nc(-c4cccc5cccnc45)n3)c(-c3ccc4ccc5cccnc5c4n3)cc2c1 ; c1ccc(-c2cccc(-c3nc(-c4cccc5ccc(-c6ccc7ccc8cccnc8c7n6)cc45)nc(-c4cccc5cccnc45)n3)c2)cc1 ; c1ccc(-c2ccc(-c3nc(-c4ccc(-c5ccc6ccc7cccnc7c6n5)cc4)nc(-c4cccc5cccnc45)n3)cc2)cc1] 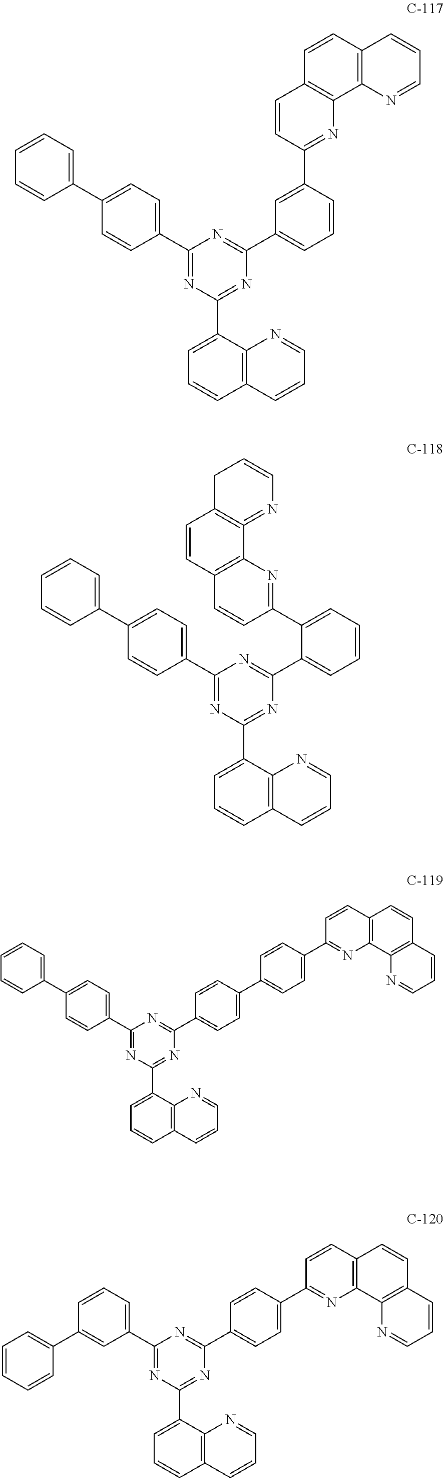 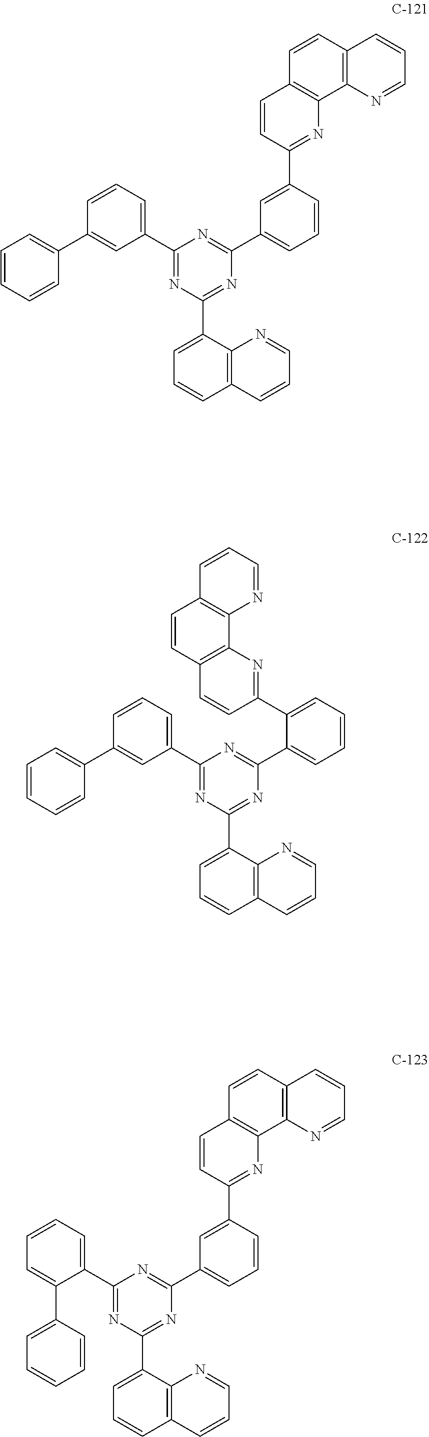 [SMILES: c1ccc(-c2cccc(-c3nc(-c4cccc(-c5ccc6ccc7cccnc7c6n5)c4)nc(-c4cccc5cccnc45)n3)c2)cc1 ; c1ccc(-c2cccc(-c3nc(-c4ccccc4-c4ccc5ccc6cccnc6c5n4)nc(-c4cccc5cccnc45)n3)c2)cc1 ; c1ccc(-c2ccccc2-c2nc(-c3cccc(-c4ccc5ccc6cccnc6c5n4)c3)nc(-c3cccc4cccnc34)n2)cc1] 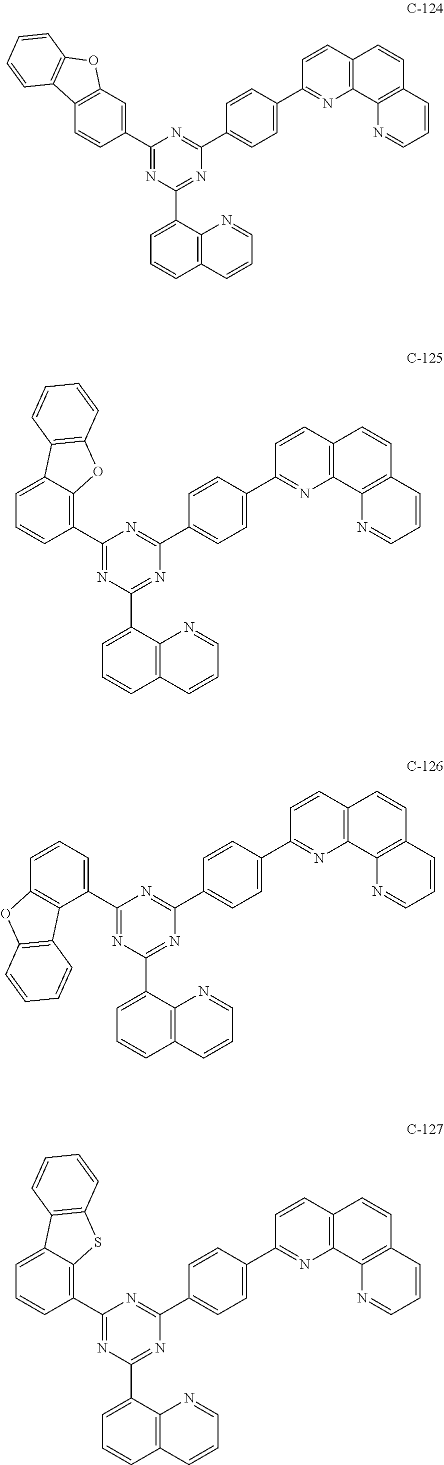 [SMILES: c1cnc2c(-c3nc(-c4ccc(-c5ccc6ccc7cccnc7c6n5)cc4)nc(-c4ccc5c(c4)oc4ccccc45)n3)cccc2c1 ; c1cnc2c(-c3nc(-c4ccc(-c5ccc6ccc7cccnc7c6n5)cc4)nc(-c4cccc5c4oc4ccccc45)n3)cccc2c1 ; c1cnc2c(-c3nc(-c4ccc(-c5ccc6ccc7cccnc7c6n5)cc4)nc(-c4cccc5oc6ccccc6c45)n3)cccc2c1 ; c1cnc2c(-c3nc(-c4ccc(-c5ccc6ccc7cccnc7c6n5)cc4)nc(-c4cccc5c4sc4ccccc45)n3)cccc2c1] 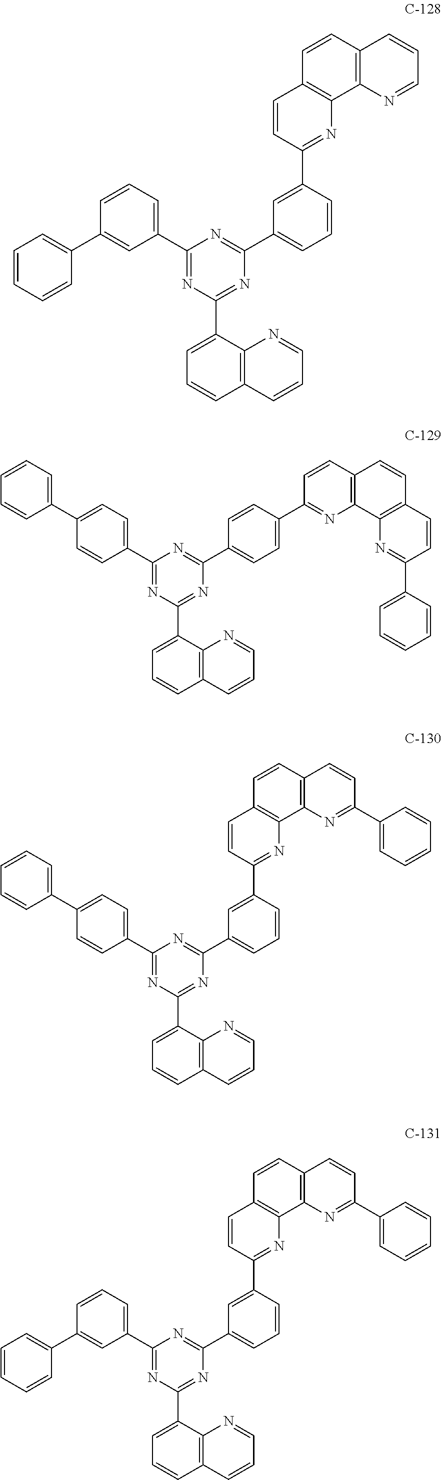 [SMILES: c1ccc(-c2cccc(-c3nc(-c4cccc(-c5ccc6ccc7cccnc7c6n5)c4)nc(-c4cccc5cccnc45)n3)c2)cc1 ; c1ccc(-c2ccc(-c3nc(-c4ccc(-c5ccc6ccc7ccc(-c8ccccc8)nc7c6n5)cc4)nc(-c4cccc5cccnc45)n3)cc2)cc1 ; c1ccc(-c2ccc(-c3nc(-c4cccc(-c5ccc6ccc7ccc(-c8ccccc8)nc7c6n5)c4)nc(-c4cccc5cccnc45)n3)cc2)cc1 ; c1ccc(-c2cccc(-c3nc(-c4cccc(-c5ccc6ccc7ccc(-c8ccccc8)nc7c6n5)c4)nc(-c4cccc5cccnc45)n3)c2)cc1] 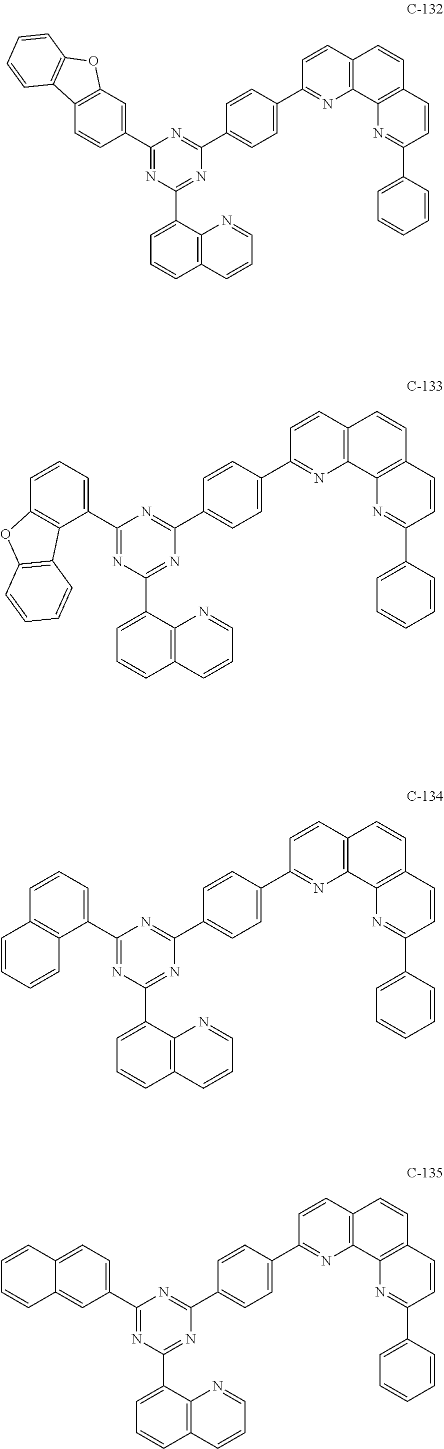 [SMILES: c1ccc(-c2ccc3ccc4ccc(-c5ccc(-c6nc(-c7ccc8c(c7)oc7ccccc78)nc(-c7cccc8cccnc78)n6)cc5)nc4c3n2)cc1 ; c1ccc(-c2ccc3ccc4ccc(-c5ccc(-c6nc(-c7cccc8cccnc78)nc(-c7cccc8oc9ccccc9c78)n6)cc5)nc4c3n2)cc1 ; c1ccc(-c2ccc3ccc4ccc(-c5ccc(-c6nc(-c7cccc8ccccc78)nc(-c7cccc8cccnc78)n6)cc5)nc4c3n2)cc1 ; c1ccc(-c2ccc3ccc4ccc(-c5ccc(-c6nc(-c7ccc8ccccc8c7)nc(-c7cccc8cccnc78)n6)cc5)nc4c3n2)cc1] 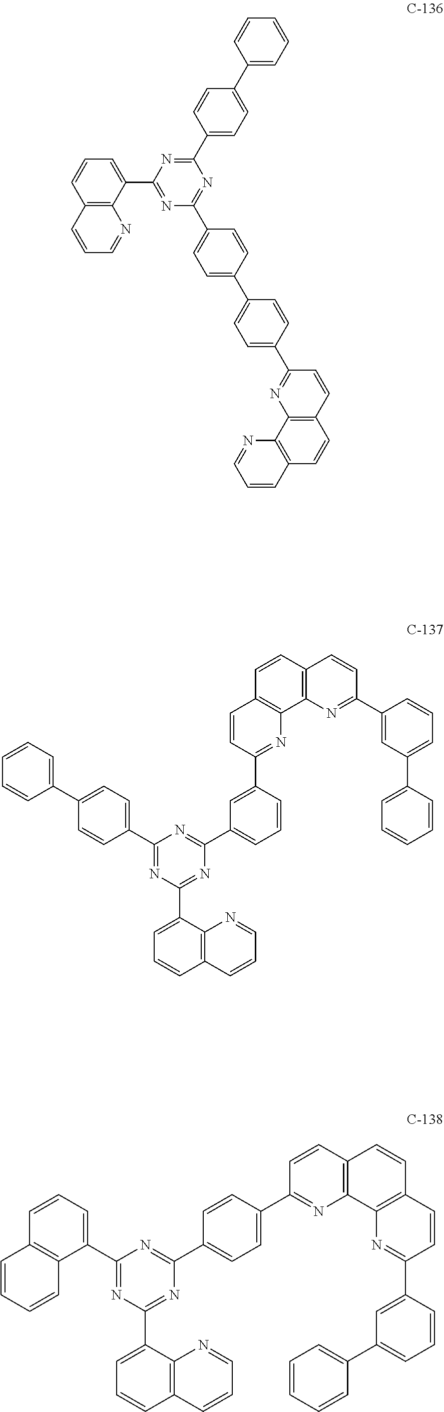 [SMILES: c1ccc(-c2ccc(-c3nc(-c4ccc(-c5ccc(-c6ccc7ccc8cccnc8c7n6)cc5)cc4)nc(-c4cccc5cccnc45)n3)cc2)cc1 ; c1ccc(-c2ccc(-c3nc(-c4cccc(-c5ccc6ccc7ccc(-c8cccc(-c9ccccc9)c8)nc7c6n5)c4)nc(-c4cccc5cccnc45)n3)cc2)cc1 ; c1ccc(-c2cccc(-c3ccc4ccc5ccc(-c6ccc(-c7nc(-c8cccc9ccccc89)nc(-c8cccc9cccnc89)n7)cc6)nc5c4n3)c2)cc1] 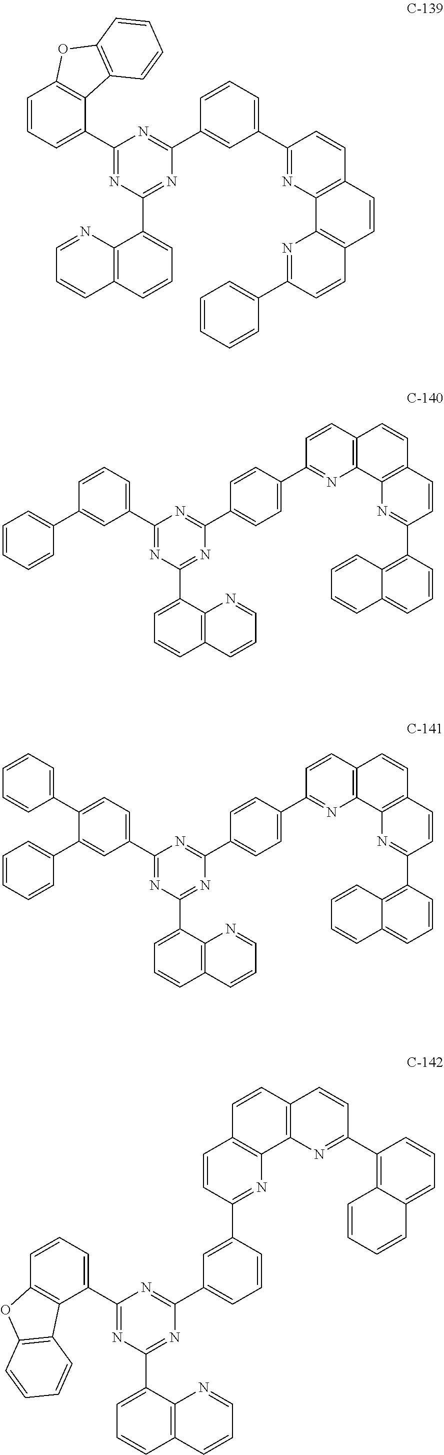 [SMILES: c1ccc(-c2ccc3ccc4ccc(-c5cccc(-c6nc(-c7cccc8cccnc78)nc(-c7cccc8oc9ccccc9c78)n6)c5)nc4c3n2)cc1 ; c1ccc(-c2cccc(-c3nc(-c4ccc(-c5ccc6ccc7ccc(-c8cccc9ccccc89)nc7c6n5)cc4)nc(-c4cccc5cccnc45)n3)c2)cc1 ; c1ccc(-c2ccc(-c3nc(-c4ccc(-c5ccc6ccc7ccc(-c8cccc9ccccc89)nc7c6n5)cc4)nc(-c4cccc5cccnc45)n3)cc2-c2ccccc2)cc1 ; c1cc(-c2ccc3ccc4ccc(-c5cccc6ccccc56)nc4c3n2)cc(-c2nc(-c3cccc4cccnc34)nc(-c3cccc4oc5ccccc5c34)n2)c1] 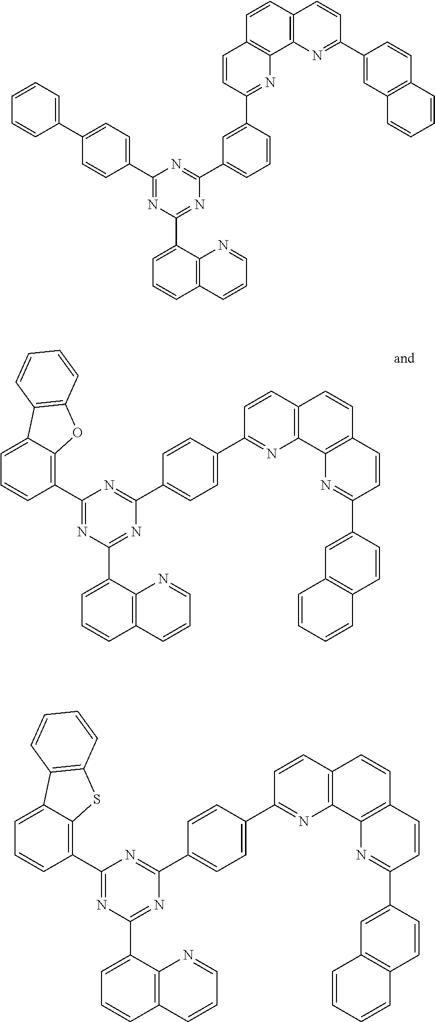 [SMILES: c1ccc(-c2ccc(-c3nc(-c4cccc(-c5ccc6ccc7ccc(-c8ccc9ccccc9c8)nc7c6n5)c4)nc(-c4cccc5cccnc45)n3)cc2)cc1 ; c1ccc2cc(-c3ccc4ccc5ccc(-c6ccc(-c7nc(-c8cccc9cccnc89)nc(-c8cccc9c8oc8ccccc89)n7)cc6)nc5c4n3)ccc2c1 ; c1ccc2cc(-c3ccc4ccc5ccc(-c6ccc(-c7nc(-c8cccc9cccnc89)nc(-c8cccc9c8sc8ccccc89)n7)cc6)nc5c4n3)ccc2c1]
wherein “Dₙ” in the above compounds means that n number of hydrogens is replaced by deuterium, wherein n is from 1 to the maximum number of hydrogen in the compound.

9. An organic electroluminescent compound represented by the following formula 4: 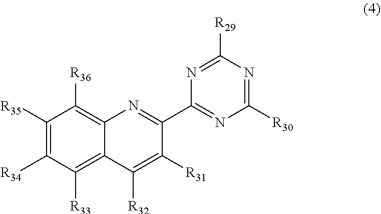
In formula 4,
R₂₉ to R₃₆ each independently represent, hydrogen, deuterium, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, or a substituted or unsubstituted tri(C6-C30)arylsilyl; and
With the proviso that neither R₂₉ nor R₃₀ is a substituted or unsubstituted fluorenyl.

10. The organic electroluminescence device according to claim 9, wherein the compound represented by formula 4 is selected from the following compounds: 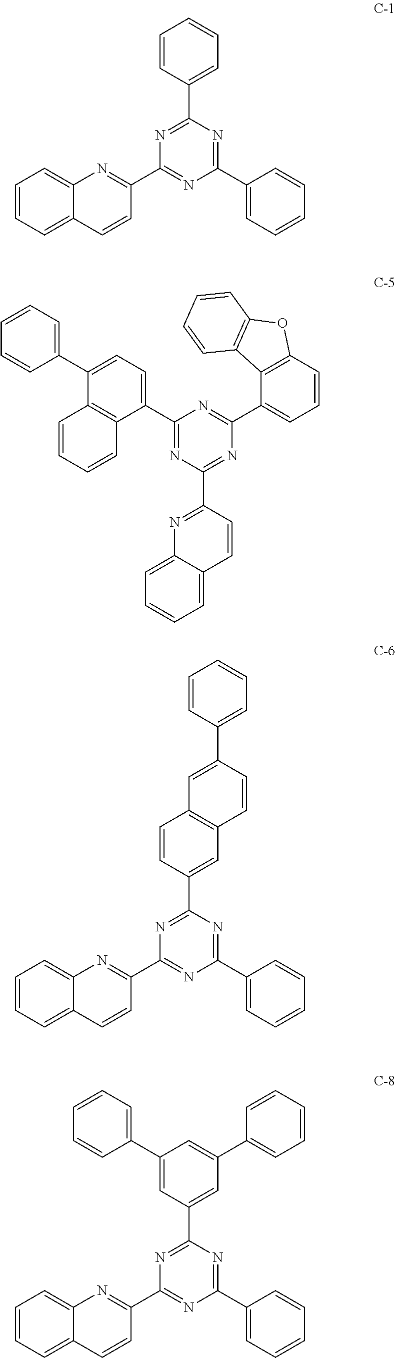 [SMILES: c1ccc(-c2nc(-c3ccccc3)nc(-c3ccc4ccccc4n3)n2)cc1 ; c1ccc(-c2ccc(-c3nc(-c4ccc5ccccc5n4)nc(-c4cccc5oc6ccccc6c45)n3)c3ccccc23)cc1 ; c1ccc(-c2ccc3cc(-c4nc(-c5ccccc5)nc(-c5ccc6ccccc6n5)n4)ccc3c2)cc1 ; c1ccc(-c2cc(-c3ccccc3)cc(-c3nc(-c4ccccc4)nc(-c4ccc5ccccc5n4)n3)c2)cc1] 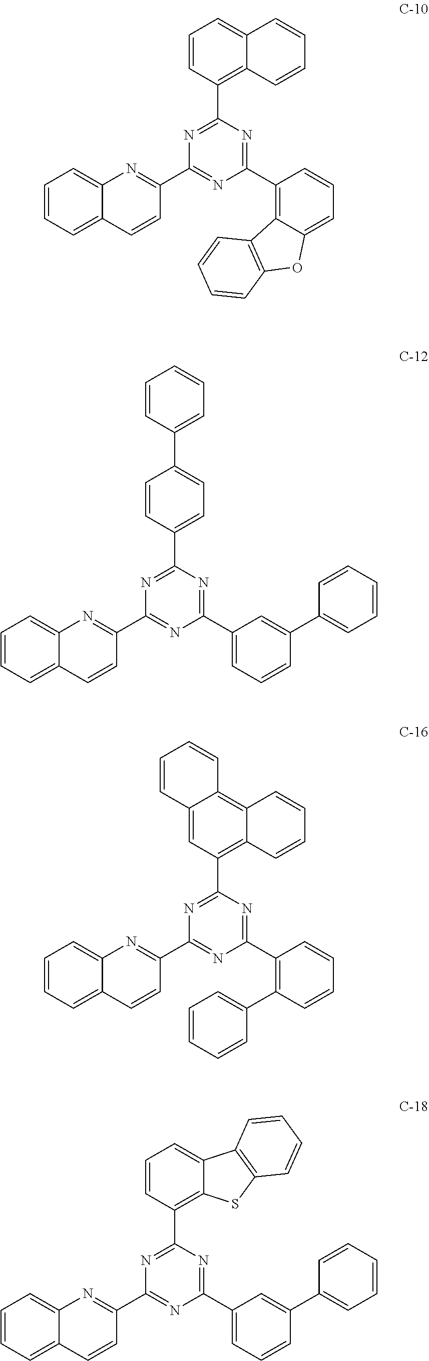 [SMILES: c1ccc2nc(-c3nc(-c4cccc5ccccc45)nc(-c4cccc5oc6ccccc6c45)n3)ccc2c1 ; c1ccc(-c2ccc(-c3nc(-c4cccc(-c5ccccc5)c4)nc(-c4ccc5ccccc5n4)n3)cc2)cc1 ; c1ccc(-c2ccccc2-c2nc(-c3ccc4ccccc4n3)nc(-c3cc4ccccc4c4ccccc34)n2)cc1 ; c1ccc(-c2cccc(-c3nc(-c4ccc5ccccc5n4)nc(-c4cccc5c4sc4ccccc45)n3)c2)cc1] 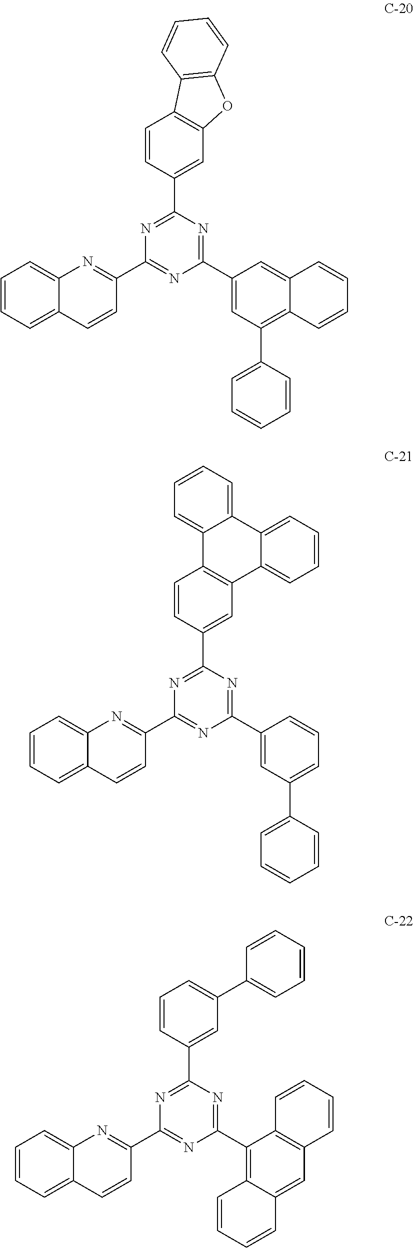 [SMILES: c1ccc(-c2cc(-c3nc(-c4ccc5c(c4)oc4ccccc45)nc(-c4ccc5ccccc5n4)n3)cc3ccccc23)cc1 ; c1ccc(-c2cccc(-c3nc(-c4ccc5c6ccccc6c6ccccc6c5c4)nc(-c4ccc5ccccc5n4)n3)c2)cc1 ; c1ccc(-c2cccc(-c3nc(-c4ccc5ccccc5n4)nc(-c4c5ccccc5cc5ccccc45)n3)c2)cc1] 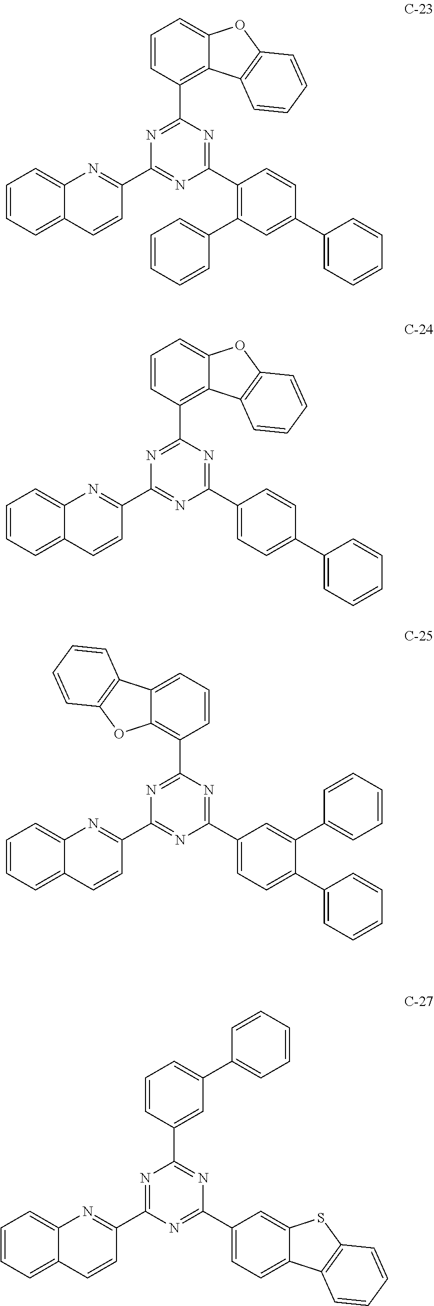 [SMILES: c1ccc(-c2ccc(-c3nc(-c4ccc5ccccc5n4)nc(-c4cccc5oc6ccccc6c45)n3)c(-c3ccccc3)c2)cc1 ; c1ccc(-c2ccc(-c3nc(-c4ccc5ccccc5n4)nc(-c4cccc5oc6ccccc6c45)n3)cc2)cc1 ; c1ccc(-c2ccc(-c3nc(-c4ccc5ccccc5n4)nc(-c4cccc5c4oc4ccccc45)n3)cc2-c2ccccc2)cc1 ; c1ccc(-c2cccc(-c3nc(-c4ccc5c(c4)sc4ccccc45)nc(-c4ccc5ccccc5n4)n3)c2)cc1] 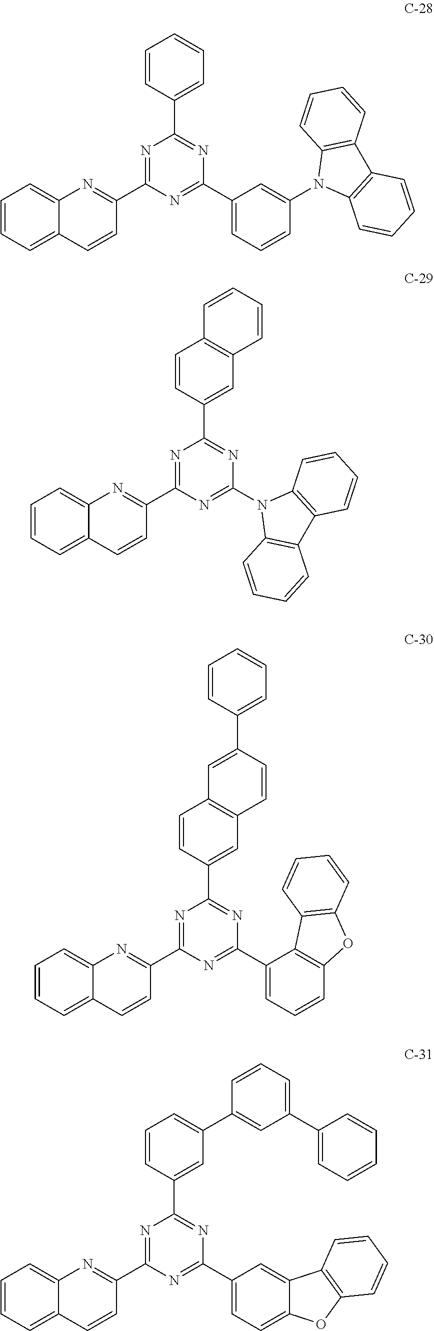 [SMILES: c1ccc(-c2nc(-c3cccc(-n4c5ccccc5c5ccccc54)c3)nc(-c3ccc4ccccc4n3)n2)cc1 ; c1ccc2cc(-c3nc(-c4ccc5ccccc5n4)nc(-n4c5ccccc5c5ccccc54)n3)ccc2c1 ; c1ccc(-c2ccc3cc(-c4nc(-c5ccc6ccccc6n5)nc(-c5cccc6oc7ccccc7c56)n4)ccc3c2)cc1 ; c1ccc(-c2cccc(-c3cccc(-c4nc(-c5ccc6oc7ccccc7c6c5)nc(-c5ccc6ccccc6n5)n4)c3)c2)cc1] 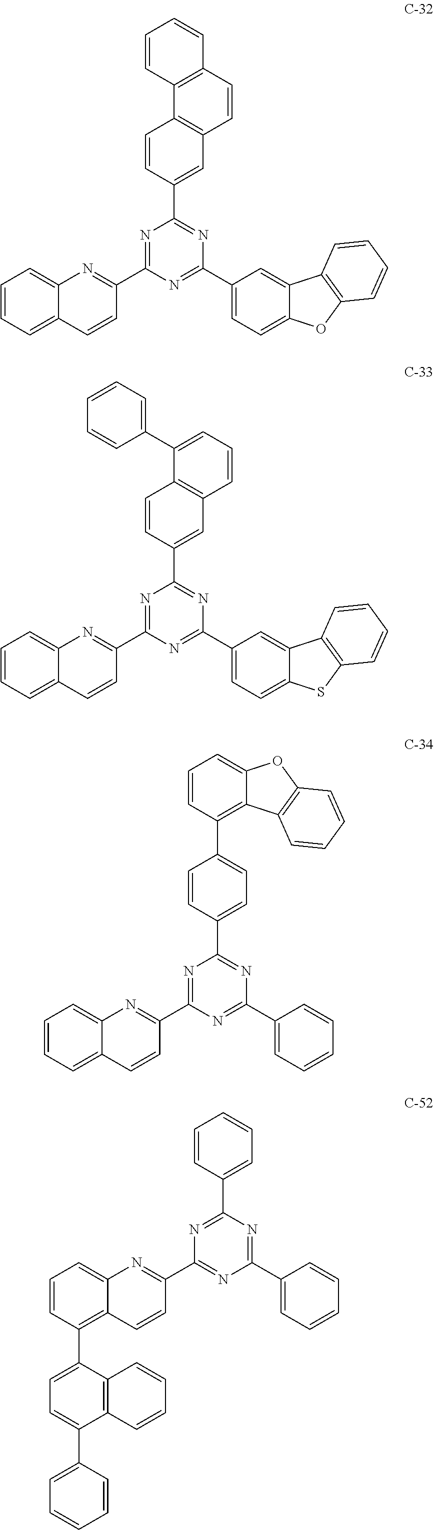 [SMILES: c1ccc2nc(-c3nc(-c4ccc5c(ccc6ccccc65)c4)nc(-c4ccc5oc6ccccc6c5c4)n3)ccc2c1 ; c1ccc(-c2cccc3cc(-c4nc(-c5ccc6sc7ccccc7c6c5)nc(-c5ccc6ccccc6n5)n4)ccc23)cc1 ; c1ccc(-c2nc(-c3ccc(-c4cccc5oc6ccccc6c45)cc3)nc(-c3ccc4ccccc4n3)n2)cc1 ; c1ccc(-c2nc(-c3ccccc3)nc(-c3ccc4c(-c5ccc(-c6ccccc6)c6ccccc56)cccc4n3)n2)cc1] 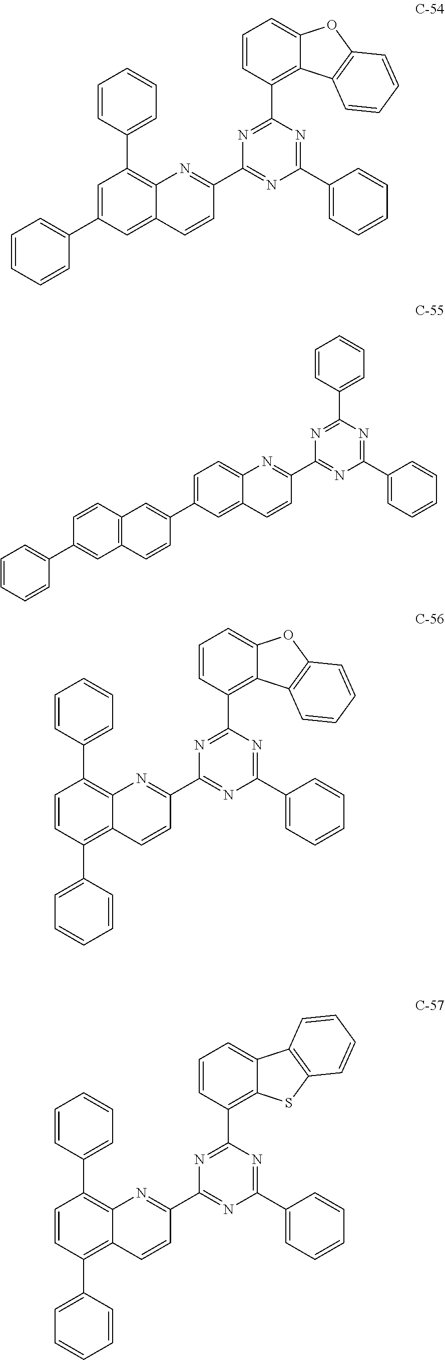 [SMILES: c1ccc(-c2cc(-c3ccccc3)c3nc(-c4nc(-c5ccccc5)nc(-c5cccc6oc7ccccc7c56)n4)ccc3c2)cc1 ; c1ccc(-c2ccc3cc(-c4ccc5nc(-c6nc(-c7ccccc7)nc(-c7ccccc7)n6)ccc5c4)ccc3c2)cc1 ; c1ccc(-c2nc(-c3ccc4c(-c5ccccc5)ccc(-c5ccccc5)c4n3)nc(-c3cccc4oc5ccccc5c34)n2)cc1 ; c1ccc(-c2nc(-c3ccc4c(-c5ccccc5)ccc(-c5ccccc5)c4n3)nc(-c3cccc4c3sc3ccccc34)n2)cc1] 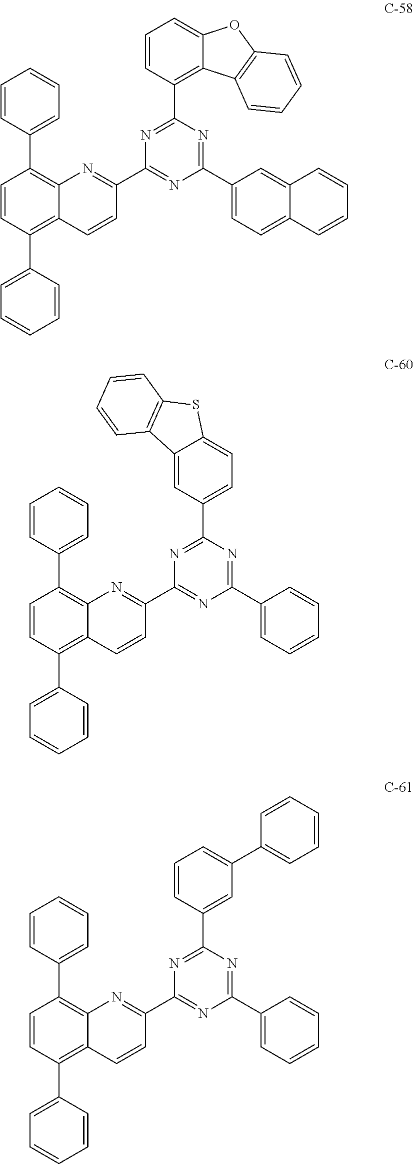 [SMILES: c1ccc(-c2ccc(-c3ccccc3)c3nc(-c4nc(-c5ccc6ccccc6c5)nc(-c5cccc6oc7ccccc7c56)n4)ccc23)cc1 ; c1ccc(-c2nc(-c3ccc4sc5ccccc5c4c3)nc(-c3ccc4c(-c5ccccc5)ccc(-c5ccccc5)c4n3)n2)cc1 ; c1ccc(-c2cccc(-c3nc(-c4ccccc4)nc(-c4ccc5c(-c6ccccc6)ccc(-c6ccccc6)c5n4)n3)c2)cc1] 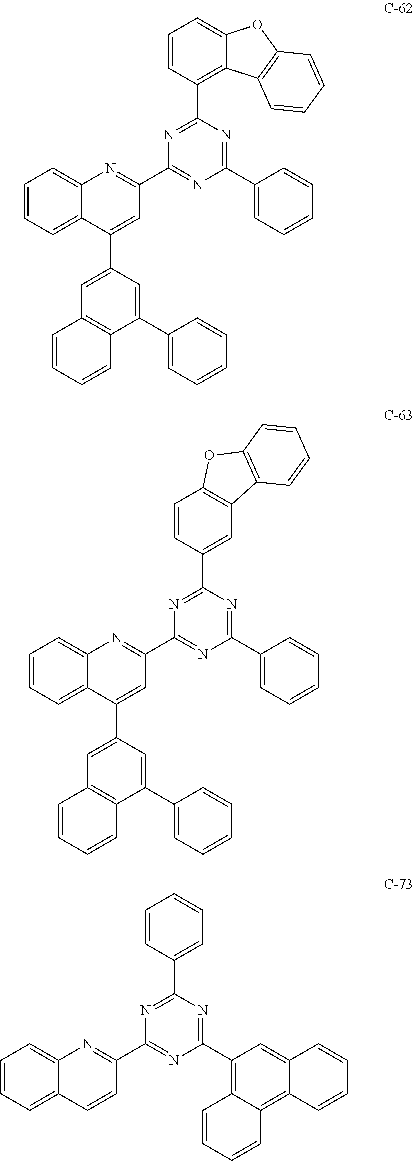 [SMILES: c1ccc(-c2nc(-c3cc(-c4cc(-c5ccccc5)c5ccccc5c4)c4ccccc4n3)nc(-c3cccc4oc5ccccc5c34)n2)cc1 ; c1ccc(-c2nc(-c3ccc4oc5ccccc5c4c3)nc(-c3cc(-c4cc(-c5ccccc5)c5ccccc5c4)c4ccccc4n3)n2)cc1 ; c1ccc(-c2nc(-c3ccc4ccccc4n3)nc(-c3cc4ccccc4c4ccccc34)n2)cc1] 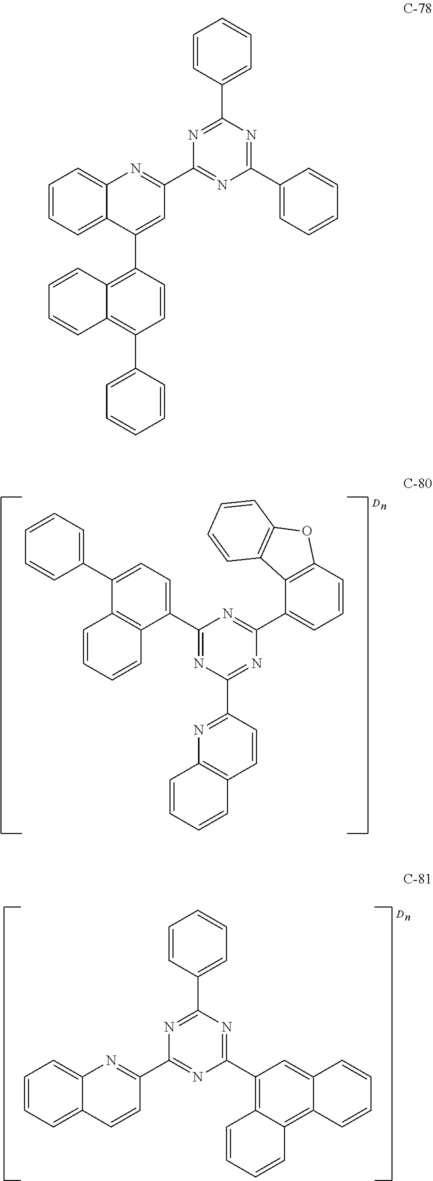 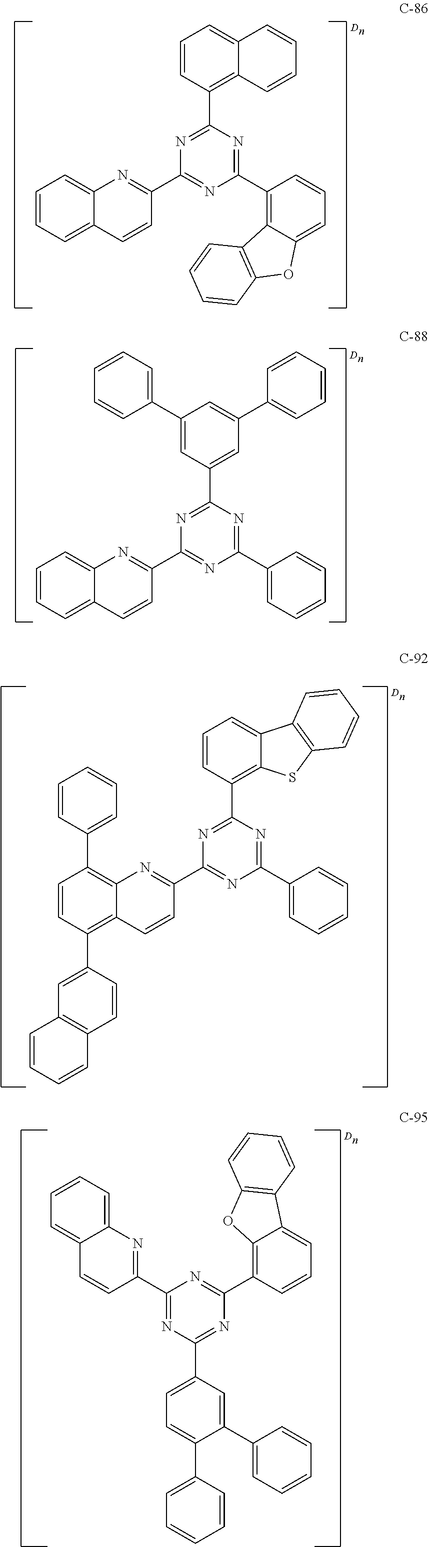 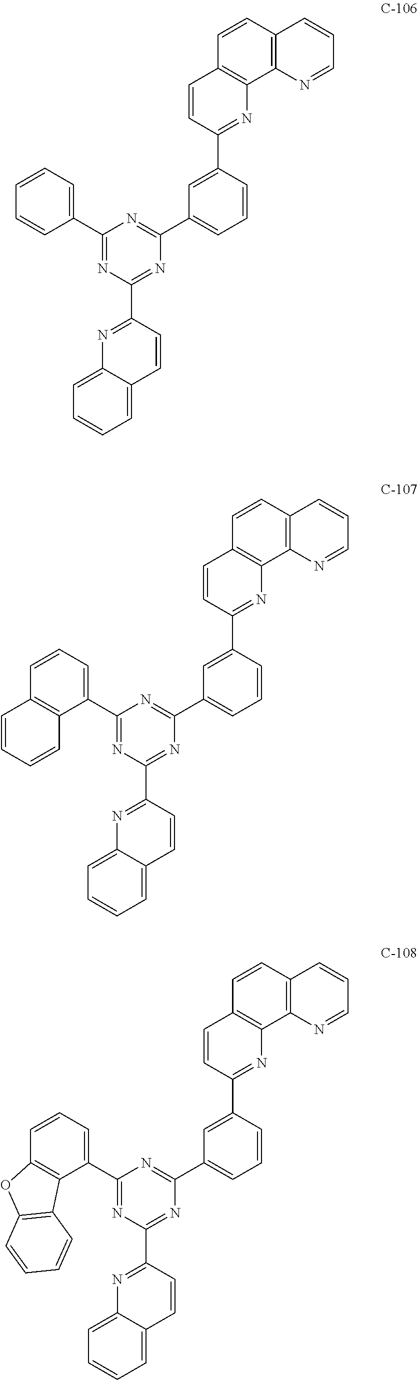 [SMILES: c1ccc(-c2nc(-c3cccc(-c4ccc5ccc6cccnc6c5n4)c3)nc(-c3ccc4ccccc4n3)n2)cc1 ; c1cc(-c2ccc3ccc4cccnc4c3n2)cc(-c2nc(-c3ccc4ccccc4n3)nc(-c3cccc4ccccc34)n2)c1 ; c1cc(-c2ccc3ccc4cccnc4c3n2)cc(-c2nc(-c3ccc4ccccc4n3)nc(-c3cccc4oc5ccccc5c34)n2)c1] 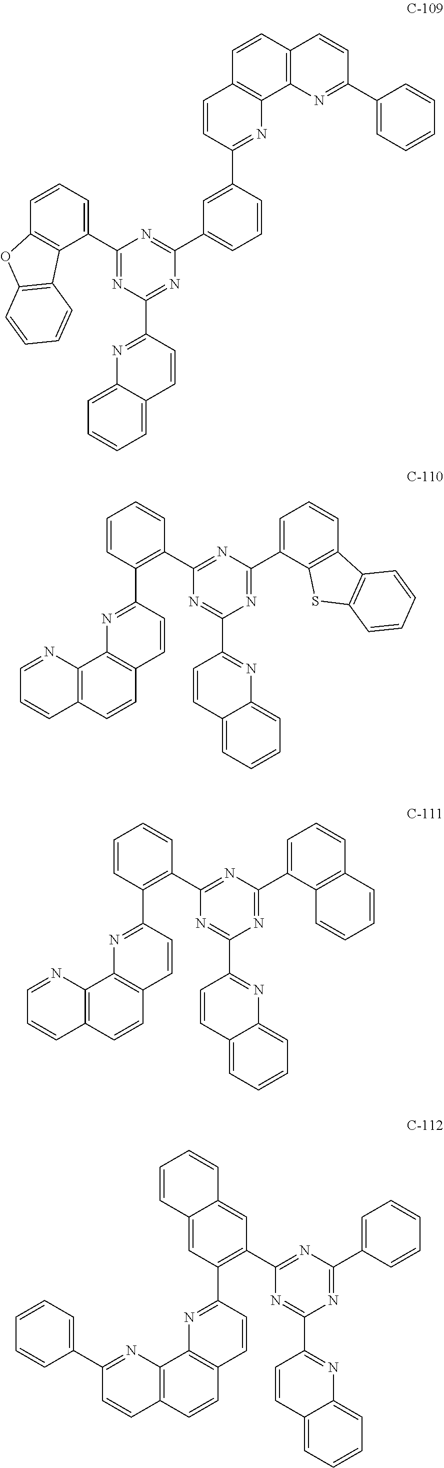 [SMILES: c1ccc(-c2ccc3ccc4ccc(-c5cccc(-c6nc(-c7ccc8ccccc8n7)nc(-c7cccc8oc9ccccc9c78)n6)c5)nc4c3n2)cc1 ; c1ccc(-c2nc(-c3ccc4ccccc4n3)nc(-c3cccc4c3sc3ccccc34)n2)c(-c2ccc3ccc4cccnc4c3n2)c1 ; c1ccc(-c2nc(-c3ccc4ccccc4n3)nc(-c3cccc4ccccc34)n2)c(-c2ccc3ccc4cccnc4c3n2)c1 ; c1ccc(-c2ccc3ccc4ccc(-c5cc6ccccc6cc5-c5nc(-c6ccccc6)nc(-c6ccc7ccccc7n6)n5)nc4c3n2)cc1] 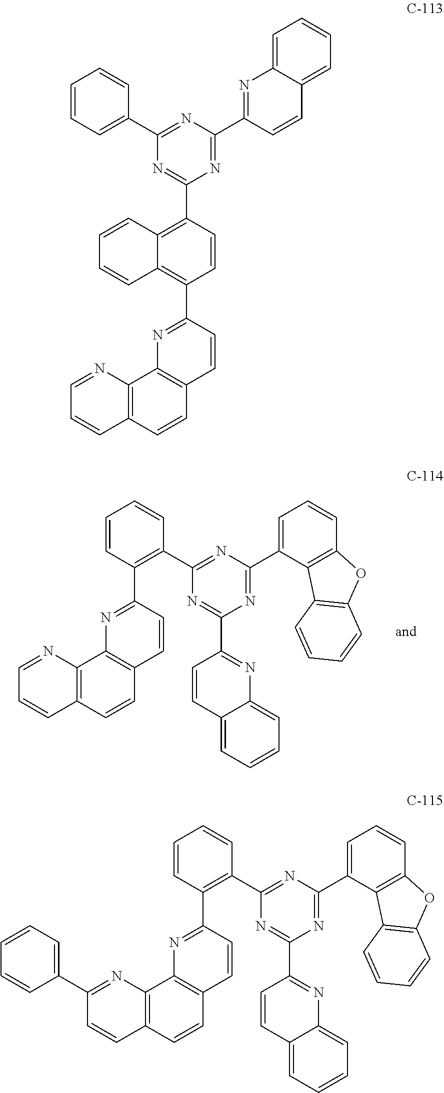 [SMILES: c1ccc(-c2nc(-c3ccc4ccccc4n3)nc(-c3ccc(-c4ccc5ccc6cccnc6c5n4)c4ccccc34)n2)cc1 ; c1ccc(-c2nc(-c3ccc4ccccc4n3)nc(-c3cccc4oc5ccccc5c34)n2)c(-c2ccc3ccc4cccnc4c3n2)c1 ; c1ccc(-c2ccc3ccc4ccc(-c5ccccc5-c5nc(-c6ccc7ccccc7n6)nc(-c6cccc7oc8ccccc8c67)n5)nc4c3n2)cc1]
wherein “Dₙ” in the above compounds means that n number of hydrogens is replaced by deuterium, wherein n is from 1 to the maximum number of hydrogen in the compound.
